(19) United States
(12) Patent Application Publication
(10) Pub. No.: US 2023/0159649 A1
(43) Pub. Date: May 25, 2023

(54) METHODS AND COMBINATIONS FOR DUAL TARGETING OF TNF FAMILY MEMBERS

(71) Applicant: La Jolla Institute for Immunology, Jolla, CA (US)

(72) Inventors: Michael Croft, La Jolla, CA (US); Rana Herro, La Jolla, CA (US)

(73) Assignee: La Jolla Institute for Immunology, La Jolla, CA (US)

(21) Appl. No.: 17/916,548

(22) PCT Filed: Apr. 1, 2021
(86) PCT No.: PCT/US2021/025453

§ 371 Date: Sep. 30, 2022

Related U.S. Application Data
(60) Provisional application No. 63/004,030, filed on Apr. 2, 2020.

(51) Int. Cl. C07K 16/28; A61P 29/00; A61P 37/00

(52) U.S. Cl. CPC C07K 16/2875; A61P 29/00; A61P 37/00; A61K 2039/507

(57) ABSTRACT

Provided herein are methods for one or more of reducing or inhibiting a fibrotic disease in a subject in need thereof; treating a skin disease or inflammation in a subject in need thereof; treating an autoimmune disorder in a subject in need thereof; treating a respiratory disease in a subject in need thereof, which comprises modulating the activity of LIGHT (p30 polypeptide) and the activity of TNF-like Ligand 1A (TL1A) in the subject. Also disclosed herein include pharmaceutical compositions, combinations, and kits for use with one or more of the methods disclosed herein.

### CROSS-REFERENCE TO RELATED APPLICATIONS

This application claims priority under 35 U.S.C. § 119(e) to U.S. Provisional Application No. 63/004,030, filed Apr. 2, 2020, the content of which is hereby incorporated by reference in its entirety.

### STATEMENT AS TO FEDERALLY SPONSORED RESEARCH

This invention was made with the support of the United States government under Contract number A1070535 by the National Institutes of Health (NIH). The Government has certain rights in this invention.

### BACKGROUND OF THE DISCLOSURE

Fibrosis and thickening of the skin is a characteristic of several inflammatory and autoimmune diseases including systemic sclerosis (SSc) or scleroderma, and atopic dermatitis (Boin and Wigley, 2009; Rosenbloom et al., 2010; Wynn and Ramalingam, 2012; Yamamoto, 2009). Current treatments involve non-selective immunotherapy with corticosteroids, D-penicillamine, methotrexate, or cyclophosphamide, but defining new targets for intervention of fibrosis in the skin is important. Fibrosis is a feature that is shared with other diseases such as severe asthma, and autoimmune diseases like RA, Crohn's disease, and SLE, but whether there are common molecules that promote clinical symptoms across these syndromes is not clear. As such, there is a need to develop additional therapeutics for addressing fibrosis, skin fibrosis, and associated diseases and indications such as autoimmune diseases and respiratory diseases.

### SUMMARY OF THE DISCLOSURE

Disclosed herein, in certain embodiments, are methods of modulating the activity of LIGHT (which stands for “homologous to lymphotoxin, exhibits inducible expression and competes with HSV glycoprotein D for binding to herpesvirus entry mediator, a receptor expressed on T lymphocytes”) (also known as TNFSF14, p30 polypeptide) and the activity of TNF-like Ligand 1A (TL1A) (also known as TNFSF15) in a subject. In some instances, the subject has a fibrotic disease, a skin disease or inflammation, an autoimmune disorder, or a respiratory disease. In some instances, also disclosed herein include pharmaceutical compositions, combinations, and kits with use of one or more of the methods described herein.

Disclosed herein, in certain embodiments, is a method for one or more of: a) reducing or inhibiting a fibrotic disease in a subject in need thereof; b) treating a skin disease or inflammation in a subject in need thereof; c) treating an autoimmune disorder in a subject in need thereof; d) treating a respiratory disease in a subject in need thereof; or e) reducing or inhibiting the activity of a LIGHT (p30 polypeptide) receptor and/or the activity of a TNF-like Ligand 1A (TL1A) receptor in a subject in need thereof comprising, consisting essentially of, or yet further consisting of modulating the activity of LIGHT (p30 polypeptide) and the activity of TNF-like Ligand 1A (TL1A) in the subject in need thereof. In some embodiments, modulating the activity of LIGHT and the activity of TL1A in the subject in need thereof comprises, consists essentially of, or yet further consists of administering to the subject a sufficient amount of a first molecule that modulates the activity of LIGHT and a second molecule that modulates the activity of TL1A. In some embodiments, modulating the activity of LIGHT and the activity of TL1A in the subject in need thereof comprises, consists essentially of, or yet further consists of reducing, decreasing, suppressing, limiting, controlling, or inhibiting the activity of LIGHT, and reducing, decreasing, suppressing, limiting, controlling, or inhibiting the activity of TL1A. In some embodiments, the first molecule comprises, consists essentially of, or yet further consists of a fusion of an immunoglobulin with a) herpesvirus entry mediator (HVEM) or b) lymphotoxin beta receptor (LTβR) polypeptide. In some embodiments, the first molecule comprises, consists essentially of, or yet further consists of a polypeptide that binds to LIGHT, HVEM, or LTβR, a peptidomimetic that modulates LIGHT, or a small molecule that modulates LIGHT. In some embodiments, the first molecule comprises, consists essentially of, or yet further consists of an antibody that binds to LIGHT, an antibody that binds to HVEM, or an antibody that binds to LTβR. In some embodiments, the second molecule comprises, consists essentially of, or yet further consists of a fusion of death receptor 3 (DR3) with an immunoglobulin, a polypeptide that binds to DR3, a fusion of DcR3 with an immunoglobulin, a peptidomimetic that modulates TL1A, or a small molecule that modulates TL1A. In some embodiments, the second molecule comprises, consists essentially of, or yet further consists of an antibody that binds to TL1A or an antibody that binds to DR3. In some embodiments, the antibody is a full-length antibody or an antigen binding fragment thereof. In some embodiments, the antibody fragment is a Fab, Fab′, F(ab′)₂, Fv, Fd, single-chain Fv (scFv), trispecific (Fab₃), bispecific (Fab₂), diabody ((V_{L}-V_{H})₂ or (V_{H}-V_{L})₂), triabody (trivalent), tetrabody (tetravalent), minibody ((scFv-CH)₂), bispecific single-chain Fv (Bis-scFv), IgGdeltaCH2, scFv-Fc, or (scFv)₂-Fc. In some embodiments, the antibody that binds to LIGHT, HVEM, or LTβR is a multispecific antibody that binds to LIGHT, HVEM, or LTβR and one or more of TL1A or DR3. In some embodiments, the multispecific antibody comprises, consists essentially of, or yet further consists of: a) a first antigen binding domain that binds to LIGHT, HVEM, or LTβR and a second antigen binding domain that binds to TL1A; b) a first antigen binding domain that binds to LIGHT, HVEM, or LTβR and a second antigen binding domain that binds to DR3; c) a first antigen binding domain that binds to LIGHT and a second antigen binding domain that binds to TL1A or DR3; d) a first antigen binding domain that binds to HVEM and a second antigen binding domain that binds to TL1A or DR3; e) a first antigen binding domain that binds to LTβR and a second antigen binding domain that binds to TL1A or DR3; f) a first antigen binding domain that binds to LIGHT and a second antigen binding domain that binds to TL1A; g) a first antigen binding domain that binds to LIGHT and a second antigen binding domain that binds to DR3; h) a first antigen binding domain that binds to HVEM and a second antigen binding domain that binds to TL1A; i) a first antigen binding domain that binds to HVEM and a second antigen binding domain that binds to DR3; j) a first antigen binding domain that binds to LTβR and a second antigen binding domain that binds to TL1A; or k) a first antigen binding domain that binds to LTβR and a second antigen binding domain that binds to DR3. In some embodiments, the multispecific antibody is a bispecific antibody, optionally a full-length antibody or an antigen binding fragment thereof. In some embodiments, the fragment of the bispecific antibody is a scFv. In some embodiments, the method comprises administering to the subject the first molecule and the second molecule. In some embodiments, the first molecule and the second molecule are administered simultaneously. In some embodiments, the first molecule and the second molecule are administered sequentially. In some embodiments, the first molecule is administered prior to administering the second molecule. In some embodiments, the first molecule is administered after administering the second molecule. In some embodiments, the first molecule is an inhibitor of LIGHT. In some embodiments, the second molecule is an inhibitor of TL1A. In some embodiments, the fibrotic disease comprises, consists essentially of, or yet further consists of fibrosis of a parenchymal organ or tissue, optionally of the lung, liver, skin, kidney, brain, heart, joints, intestine, or the bone marrow. In some embodiments, the fibrotic disease comprises, consists essentially of, or yet further consists of interstitial lung disease (ILD), liver cirrhosis, or idiopathic pulmonary fibrosis. In some embodiments, the skin disease or inflammation comprises, consists essentially of, or yet further consists of atopic dermatitis, scleroderma, psoriasis, onchocercal dermatitis, nephrogenic fibrosing dermopathy, mixed connective tissue disease, scleromyxedema, keloid, sclerodactyly, or eosinophilic fasciitis. In some embodiments, the respiratory disease or disorder comprises, consists essentially of, or yet further consists of asthma, allergic asthma, bronchiolitis, pleuritis, chronic obstructive pulmonary disease (COPD), extrinsic bronchial asthma, allergic rhinitis, eosophageal allergy, or gastrointestinal allergy. In some embodiments, the respiratory disease comprises, consists essentially of, or yet further consists of nodules, eosinophilia, rheumatism, dermatitis and swelling (NERDS). In some embodiments, the respiratory disease comprises, consists essentially of, or yet further consists of airway obstruction, apnea, asbestosis, atelectasis, berylliosis, bronchiectasis, bronchiolitis, bronchiolitis obliterans organizing pneumonia, bronchitis, bronchopulmonarydysplasia empyema, pleural empyema, pleural epiglottitis, hemoptysis, hypertension, kartagener syndrome, meconium aspiration, pleural effusion, pleurisy, pneumonia, pneumothorax, respiratory distress syndrome, respiratory hypersensitivity, respiratory tract infections, rhinoscleroma, scimitar syndrome, severe acute respiratory syndrome, silicosis, or tracheal stenosis. In some embodiments, the autoimmune disorder is systemic sclerosis, rheumatoid arthritis (RA), lupus (e.g., systemic lupus erythematosus or SLE), inflammatory bowel disease (IBD), eosinophilic esophagitis (EoE), ankylosing spondylitis (AS), experimental autoimmune encephalomyelitis (EAE), or an autoimmune inflammatory disease of the central nervous system (CNS). In some embodiments, the method further comprises, consists essentially of, or yet further consists of administering to the subject an additional therapeutic agent. In some embodiments, the additional therapeutic agent comprises, consists essentially of, or yet further consists of an anti-inflammatory drug, a steroid, a hormone, or an immunosuppressant drug. In some embodiments, the first molecule and/or the second molecule and the additional therapeutic agent are administered simultaneously. In some embodiments, the first molecule and/or the second molecule and the additional therapeutic agent are administered sequentially. In some embodiments, the first molecule and/or the second molecule are administered prior to administering the additional therapeutic agent. In some embodiments, the first molecule and/or the second molecule are administered after administering the additional therapeutic agent. In some embodiments, the first molecule and/or the second molecule and/or the additional therapeutic agent are administered systemically. In some embodiments, the first molecule and/or the second molecule and/or the additional therapeutic agent are administered locally. In some embodiments, the first molecule and/or the second molecule and/or the additional therapeutic agent are administered by parenteral administration. In some embodiments, the first molecule and/or the second molecule and/or the additional therapeutic agent are administered intravenously or subcutaneously. In some embodiments, the subject is a mammal or a human.

Disclosed herein, in certain embodiments, is a pharmaceutical composition comprising, consisting essentially of, or yet further consisting of as the active agent a first molecule that modulates the activity of LIGHT (p30 polypeptide), a second molecule that modulates the activity of TNF-like Ligand 1A (TL1A), and a pharmaceutically acceptable excipient.

Disclosed herein, in certain embodiments, is a combination comprising, consisting essentially of, or yet further consisting of a first molecule that modulates the activity of LIGHT (p30 polypeptide) and a second molecule that modulates the activity of TNF-like Ligand 1A (TL1A). In some embodiments, the combination further comprises, consists essentially of, or yet further consists of a pharmaceutically acceptable excipient. In some embodiments, the first molecule comprises, consists essentially of, or yet further consists of a fusion of an immunoglobulin and a) herpesvirus entry mediator (HVEM) or b) lymphotoxin beta receptor (LTβR) polypeptide with. In some embodiments, the first molecule comprises, consists essentially of, or yet further consists of a polypeptide that binds to LIGHT, HVEM, or LTβR, a peptidomimetic that modulates LIGHT, or a small molecule that modulates LIGHT. In some embodiments, the first molecule comprises, consists essentially of, or yet further consists of an antibody that binds to LIGHT, an antibody that binds to HVEM, or an antibody that binds to LTβR. In some embodiments, the second molecule comprises, consists essentially of, or yet further consists of a fusion of DR3 with an immunoglobulin, a polypeptide that binds to DR3, a fusion of DcR3 with an immunoglobulin, a peptidomimetic that modulates TL1A, or a small molecule that modulates TL1A. In some embodiments, the second molecule comprises, consists essentially of, or yet further consists of an antibody that binds to TL1A or an antibody that binds to DR3. In some embodiments, antibody that binds to LIGHT, HVEM, or LTBR is a multispecific antibody that binds to LIGHT, HVEM, or LTBR and one or more of TL1A or DR3. In some embodiments, the multispecific antibody comprises, consists essentially of, or yet further consists of: a) a first antigen binding domain that binds to LIGHT, HVEM, or LTβR and a second antigen binding domain that binds to TL1A; b) a first antigen binding domain that binds to LIGHT, HVEM, or LTβR and a second antigen binding domain that binds to DR3; c) a first antigen binding domain that binds to LIGHT and a second antigen binding domain that binds to TL1A or DR3; d) a first antigen binding domain that binds to HVEM and a second antigen binding domain that binds to TL1A or DR3; e) a first antigen binding domain that binds to LTβR and a second antigen binding domain that binds to TL1A or DR3; f) a first antigen binding domain that binds to LIGHT and a second antigen binding domain that binds to TL1A; g) a first antigen binding domain that binds to LIGHT and a second antigen binding domain that binds to DR3; h) a first antigen binding domain that binds to HVEM and a second antigen binding domain that binds to TL1A; i) a first antigen binding domain that binds to HVEM and a second antigen binding domain that binds to DR3; j) a first antigen binding domain that binds to LTβR and a second antigen binding domain that binds to TL1A; or k) a first antigen binding domain that binds to LTβR and a second antigen binding domain that binds to DR3. In some embodiments, the combination comprises, consists essentially of, or yet further consists of a pharmaceutical composition comprising the first molecule that modulates the activity of LIGHT (p30 polypeptide), the second molecule that modulates the activity of TNF-like Ligand 1A (TL1A), and a pharmaceutically acceptable excipient. In some embodiments, the first molecule is an inhibitor of LIGHT. In some embodiments, the second molecule is an inhibitor of TL1A.

Disclosed herein, in certain embodiments, is a kit comprising, consisting essentially of, or yet consisting of a first molecule that modulates the activity of LIGHT (p30 polypeptide) and/or a second molecule that modulates the activity of TNF-like Ligand 1A (TL1A) for use with the method described below, and optionally comprises instructions for use. In some embodiments, the first molecule is an inhibitor of LIGHT. In some embodiments, the second molecule is an inhibitor of TL1A.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a cartoon representation of key modulators of TNF family proteins in several immune-mediated disorders. LIGHT, (also known as TNFSF14 and CD258), is a soluble and membrane expressed pro-inflammatory molecule that acts through two receptors, the herpesvirus entry mediator (HVEM; TNFRSF14; CD270) and the lymphotoxin β receptor (LTβR; TNFRSF3). HVEM is found on most lymphoid and several non-lymphoid cells, and LTβR is on APC and non-lymphoid cells. LIGHT is a product of T cells and is made by all CD4 T cells regardless of Th phenotype as well as by CD8 T cells. Moreover, LIGHT can be made by macrophages, neutrophils, and eosinophils. TL1A (TNFSF15) is also a soluble and membrane-expressed molecule, made by several cells including dendritic cells, macrophages, fibroblasts, epithelial cells, neutrophils and eosinophils. TL1A acts through the receptor DR3 (TNFRSF25) that can be expressed on T cells, innate lymphoid cells, and structural cells such as fibroblasts and epithelial cells.

FIG. 2A-FIG. 2C illustrate LTβR, HVEM, and DR3 are co-expressed or active in lung epithelial cells and fibroblasts, and keratinocytes and dermal fibroblasts. Cultured normal human bronchial epithelial cells and lung fibroblasts, and epidermal keratinocytes and dermal fibroblasts, were stained for expression of (FIG. 2A) LTβR and HVEM, (FIG. 2B) DR3 (isotype controls, gray), or (FIG. 2C) p-p65/RelA NF-κB after culture with 25 ng/ml rTL1A or PBS. Data representative of 2-4 experiments each.

FIG. 3A-FIG. 3C illustrate that rLIGHT and rTL1A promote airway remodeling independent of one another. Naïve mice were injected with PBS or 10 μg rLIGHT or rTL1A intratracheally on two consecutive days. On day 3, accumulation of collagen was analyzed in the airways with trichrome staining. (FIG. 3A) rLIGHT was injected into DR3−/− mice, or rTL1A into LIGHT−/− mice. (FIG. 3B) rLIGHT was combined with rTL1A (5 μg each), and the extent of collagen deposition scored as well as the extent of inflammation scored. (FIG. 3C) rLIGHT and rTL1A were injected into RAG−/− mice. Note: αSMA expression was also upregulated by rLIGHT and rTL1A in all cases and not shown in the data.

FIG. 4 illustrates blocking LIGHT interactions reduces airway fibrosis and remodeling induced by allergen, in a model of asthma. WT mice were sensitized with house dust mite extract (HDM) i.n. on days 0, 7 and 14, and treated 2×per week for 4 weeks with i.n. HDM to promote remodeling. Control IgG or LTβR-Ig were given i.p. 2×per week for the last 4 weeks. Airway sections stained with trichrome or antibody to αSMA.

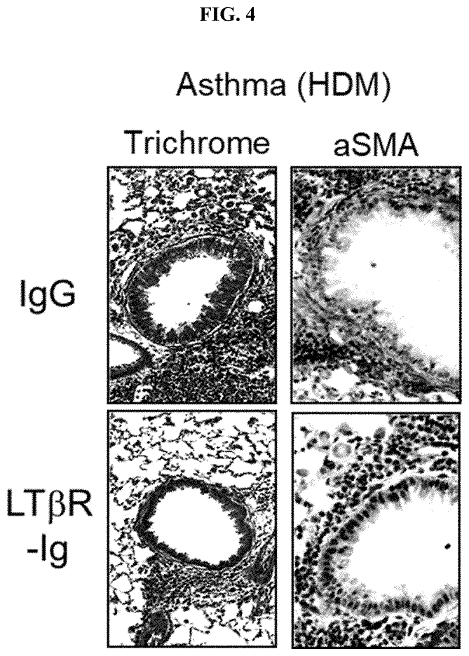

FIG. 5A-FIG. 5B illustrate reduced atopic dermatitis and psoriasis skin inflammatory responses in LIGHT-deficient mice. WT littermates and LIGHT−/− mice were sensitized: (FIG. 5A) in 2 cycles epicutaneously with house dust mite (HDM) extract, given on a gauze pad placed on the abraded shaved back for 3 days starting on day 0, and repeated on day 8; and (FIG. 5B) with a cream containing imiquimod, given on the shaved back once a day for 7 days. Mice were examined visually on day 14 (FIG. 5A) or day 8 (FIG. 5B), and with Masson's trichrome staining of skin sections (for collagen). Representative of 6-20 mice/group from 2-4 experiments.

FIG. 6A-FIG. 6C illustrate reduced airway remodeling in models of asthma and systemic sclerosis and skin remodeling in models of atopic dermatitis and psoriasis after neutralizing TL1A-DR3. (FIG. 6A-FIG. 6B) Mice were sensitized with (FIG. 6A) HDM i.n. over 6 weeks, and (FIG. 6B) bleomycin i.t over 7 days. WT and DR3−/− mice, and (FIG. 6A) WT mice treated with DR3.Fc given i.p. 2×per week for the last 4 weeks. (FIG. 6C) WT and DR3−/− mice treated epicutaneously with HDM (top), or imiquimod over 7 days (bottom). Sections stained with Masson's trichrome or anti-αSMA (bronchiole's outlined with dotted lines). Airway sections scored for trichome and αSMA expression (FIG. 6A-FIG. 6B). Data representative of, or means±s.e.m from, 6-20 mice. All results representative of, or from, 3 experiments. *p<0.05.

FIG. 7 illustrates blocking TL1A reduces airway fibrosis and remodeling in a model of systemic sclerosis. Mice were sensitized with bleomycin i.t over 7 days, and treated with DR3.Fc or control IgG given i.p. Lung sections stained with Masson's trichrome or anti-αSMA. Airway sections scored for trichome and αSMA expression. Data representative of, or means±s.e.m from, 6-20 mice.

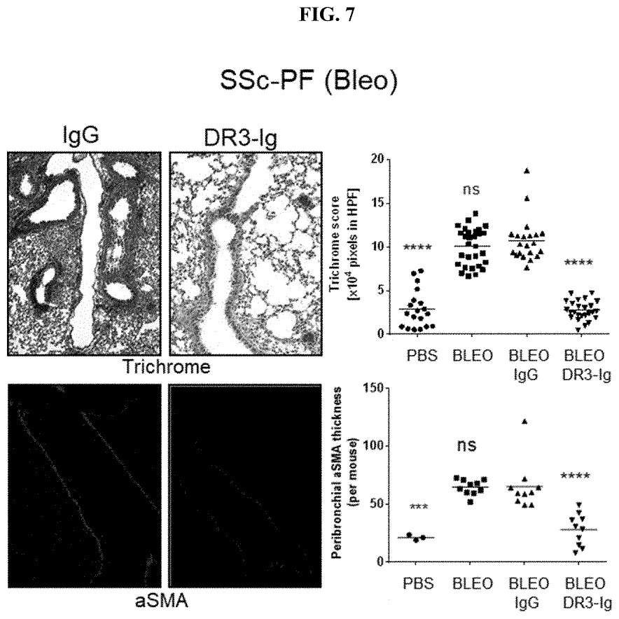

FIG. 8A illustrates LIGHT promotes steroid-resistant responses in normal human bronchial epithelial cells (HBE). HBE were stimulated with rLIGHT without (black bars) or with (gray bars) budesonide. Data are mean fold increase in mRNA over unstimulated cells after 3 days, measured by RT-PCR. Steroid sensitivity was arbitrarily set at >75% inhibition of the LIGHT-induced response, indicated to the left of the thick dotted lines.

FIG. 8B illustrates TL1A promotes fibrotic activity in human bronchial epithelial cells similar to LIGHT. HBE were stimulated with rLIGHT (left) or rTL1A (right) for 48 hrs and expression of periostin and TSLP examined by IF staining.

FIG. 9A-FIG. 9D illustrate LIGHT and TL1A induce inflammatory and remodeling activity in human airway fibroblasts (HAF). HAF were stimulated with rLIGHT or rTL1A with or without TGF-β. (FIG. 9A) Proliferation (left, thymidine incorporation) or αSMA mRNA (right) induced by LIGHT, TGF-β or both. (FIG. 9B) mRNA for select genes induced by LIGHT. (FIG. 9C) Collagen 13 and periostin protein (IF stain; DAPI is used for collagen) and mRNA induced by TL1A. (FIG. 9D) Proliferation (left, thymidine incorporation) and αSMA mRNA (right) induced by TL1A, TGF-β, or their combination. Data individual cultures or means of triplicates. mRNA either fold increase over unstimulated cells or relative to GAPDH.

FIG. 10 illustrates therapeutic blocking of LIGHT and TL1A limits allergen-driven atopic dermatitis. WT mice were sensitized epicutaneously with HDM in 2 cycles. Mice were treated with IgG, DR3.Fc, LTβR.Fc, or both (200 μg i.v.) after disease had developed, starting on day 7 one day prior to the second HDM exposure, with mice analyzed on day 14. Clinical symptoms (eruption, scaling, bleeding, redness) from individual mice; and representative skin sections stained with trichrome blue. Data individual mice, with 4 mice per group. *p<0.05. **p<0.01. *****p<0.001.

FIG. 11A-FIG. 11B illustrate LIGHT and TL1A and their receptors are expressed in atopic dermatitis (AD) skin. FIG. 11A shows bulk RNA-seq analysis of transcripts in AD lesional and non-lesional skin vs. healthy. IL-5 and IL-9 are expressed at low levels (TPM, transcripts per million, <0.3) like IL-4 (not shown); IL-13 is upregulated in lesional and non-lesional AD; LIGHT is expressed at higher levels than IL-13 in AD lesions (TPM mean 1.2 vs. 0.9) and is upregulated in lesional and non-lesional skin compared to healthy; TL1A is expressed at levels almost equivalent to IL-13 (TPM mean 0.6 vs 0.9) albeit it is not statistically upregulated as healthy skin expresses high TL1A transcripts (TPM mean 0.5); LTβR, HVEM, and DR3 are all strongly expressed in AD lesional skin (TPM means, 23, 7, and 14, respectively) as well as in healthy skin (TPM means 14, 4, 13); LTβR and HVEM are upregulated in lesional and non-lesional skin, whereas DR3 is only up in non-lesional skin. FIG. 11B shows single cell RNA-seq analysis of transcripts in AD lesional and non-lesional skin. Data are mean TPM from multiple single cells. LTβR, HVEM, and DR3 transcripts are found in keratinocytes and fibroblasts from lesional and non-lesional skin.

FIG. 12A and FIG. 12B illustrate LIGHT-deficiency decreases skin fibrosis and remodeling induced by bleomycin in a model of scleroderma, whereas LIGHT intradermal injection alone induces skin fibrosis and remodeling reminiscent of scleroderma. FIG. 12A shows WT and LIGHT mice were injected with bleomycin. FIG. 12B shows WT mice were injected with either PBS or LIGHT (20 μg/mouse). Masson trichrome (collagen), alpha Smooth muscle actin (αSMA) and TSLP immunofluorescent staining of skin sections. The data is representative of 6 to 10 mice per group.

FIG. 13 shows unregulated expression of LIGHT and TL1A mRNA transcripts in lung biopsies from patients with systemic sclerosis with pulmonary fibrosis compared to normal lungs from healthy individuals.

### DETAILED DESCRIPTION OF THE DISCLOSURE

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (—) 15%, 10%, 5%, 3%, 2%, or 1%.

LIGHT (TNFSF14, p30 polypeptide) is a protein expressed by activated CD4/CD8 T cells, dendritic cells (DCs), monocytes, and natural killer cells (NK). The binding of LIGHT to herpes virus entry mediator (HVEM), which is expressed on resting T cells, DCs, and monocytes, non-lymphoid cells, or the lymphotoxin beta receptor (LTβR), which is expressed on several lymphoid and non-lymphoid cells, promotes cell activation, proliferation, and/or production of soluble mediators such as cytokines.

As used herein, a first molecule that modulates the activity of a LIGHT ((p30 polypeptide) receptor refers to a molecule that alters the activity of the LIGHT receptor. In some instances, the first molecule decreases, reduces, or limits the activity of the LIGHT receptor, e.g., decreases the activity by about 10%, 20%, 30%, 40%, 50%, 60%, 70%, 80, 90%, or 100%. In some instances, the first molecule decreases the activity of the LIGHT receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or more. The first molecule can modulate the activity of the LIGHT receptor either directly or indirectly, by binding to LIGHT, or to its binding partner HVEM or LTβR. Modulators of the LIGHT receptor include, but are not limited to, a fusion of an immunoglobulin with HVEM or LTβR; a polypeptide that binds to LIGHT, HVEM, or LTβR; a peptidomimetic that modulates LIGHT; a small molecule that modulates LIGHT; an antibody that binds to LIGHT, an antibody that binds to HVEM, or an antibody that binds to LTβR.

In some embodiments, the first molecule that modulates the activity of the LIGHT receptor is an inhibitor of LIGHT. As used herein, the term “an inhibitor of LIGHT” refers to a molecule that directly or indirectly inhibits or blocks binding of LIGHT (p30 polypeptide) to HVEM or to LTβR. Inhibitors therefore include molecules that bind to LIGHT as well as molecules that bind to a LIGHT receptor, e.g., HVEM or LTβR. LIGHT (p30 polypeptide) inhibitors therefore include molecules that bind to LIGHT (p30 polypeptide), molecules that bind to HVEM, as well as molecules that bind to LTβR. In some cases, inhibitors of LIGHT include, but are not limited to, a fusion of an immunoglobulin with HVEM or LTβR; a polypeptide that binds to LIGHT, HVEM, or LTβR; a peptidomimetic that modulates LIGHT, HVEM, or LTβR; a small molecule that modulates LIGHT; an antibody that binds to LIGHT, an antibody that binds to HVEM, or an antibody that binds to LTβR.

In some embodiments, the first molecule that modulates the activity of a LIGHT receptor comprises a fusion of an immunoglobulin with a herpesvirus entry mediator (HVEM) polypeptide or a lymphotoxin beta receptor (LTβR) polypeptide. HVEM (also known as tumor necrosis factor receptor superfamily member 14, TNFRSF14, TR2, CD270, CD40-like protein, or LIGHTR) is a member of the TNF receptor superfamily, that functions in signal transduction pathways that activate inflammatory immune responses. In some instances, the HVEM polypeptide comprises a mammalian HVEM polypeptide, e.g., a HVEM polypeptide from a rodent, a non-human primate, or a human. In some cases, the HVEM polypeptide comprises a full-length polypeptide. In other cases, the HVEM polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the fusion polypeptide comprising an immunoglobulin and a HVEM polypeptide is an inhibitor of LIGHT.

A non-limiting representative example of human HVEM (herpesvirus entry mediator) sequence comprises a polypeptide as set forth below (SEQ ID NO:1):

MEPPGDWGPPPWRSTPKTDVLRLVLYLTFLGAPCYAPALPSCKEDEYPV
GSECCPKCSPGYRVKEACGELTGTVCEPCPPGTYIAHLNGLSKCLQCQM
CDPAMGLRASRNCSRTENAVCGCSPGHFCIVQDGDHCAACRAYATSSPG
QRVQKGGTESQDTLCQNCPPGTFSPNGTLEECQHQTKCSWLVTKAGAGT
SSSHWVWWFLSGSLVIVIVCSTVGLIICVKRRKPRGDVVKVIVSVQRKR
QEAEGEATVIEALQAPPDVTTVAVEETIPSFTGRSPNH,
or an equivalent thereof.

LTβR (also known as tumor necrosis factor receptor superfamily member 3 or TNFRSF3) is a member of the TNF receptor superfamily and is a cell surface receptor for LIGHT as well as lymphotoxin alphabeta that is involved in inflammatory immune responses. In some instances, the LTβR polypeptide comprises a mammalian LTβR polypeptide, e.g., a LTβR polypeptide from a rodent, a non-human primate, or a human. In some cases, the LTβR polypeptide comprises a full-length polypeptide. In other cases, the LTβR polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the fusion polypeptide comprising an immunoglobulin and a LTβR polypeptide is an inhibitor of LIGHT.

A non-limiting representative example of human LTβR sequence comprises a polypeptide as set forth below (SEQ ID NO:2):

MLLPWATSAPGLAWGPLVLGLFGLLAASQPQAVPPYASENQTCRDQEKEY
YEPQHRICCSRCPPGTYVSAKCSRIRDTVCATCAENSYNEHWNYLTICQL
CRPCDPVMGLEEIAPCTSKRKTQCRCQPGMFCAAWALECTHCELLSDCPP
GTEAELKDEVGKGNNHCVPCKAGHFQNTSSPSARCQPHTRCENQGLVEAA
PGTAQSDTTCKNPLEPLPPEMSGTMLMLAVLLPLAFFLLLATVFSCIWKS
HPSLCRKLGSLLKRRPQGEGPNPVAGSWEPPKAHPYFPDLVQPLLPISGD
VSPVSTGLPAAPVLEAGVPQQQSPLDLTREPQLEPGEQSQVAHGTNGIHV
TGGSMTITGNIYIYNGPVLGGPPGPGDLPATPEPPYPIPEEGDPGPPGLS
TPHQEDGKAWHLAETEHCGATPSNRGPRNQFITHD,
or an equivalent thereof.

In some embodiments, the immunoglobulin is an antibody (e.g., an IgG, IgM, IgA, IgG, or IgE isotype). In some instances, the immunoglobulin is an IgG antibody (e.g., IgG1, IgG2, IgG3, or IgG4 antibody). In some instances, the immunoglobulin comprises the Fc region of an antibody (e.g., the Fc region of an IgG antibody, optionally an IgG1, IgG2, IgG3, or IgG4 Fc region). In some cases, the Fc region is from a human antibody. (e.g., a human IgG antibody, optionally a human IgG1, IgG2, IgG3, or IgG4 Fc region). An exemplary sequence of the Fc region comprises

(SEQ ID NO: 3)
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPGK,
or an equivalent thereof.

In some embodiments, the fusion polypeptide further comprises a linker that bridges the immunoglobulin with either HVEM or LTβR. In some instances, the linker comprises a peptide, e.g., a poly-Ala peptide, a poly-Gly peptide, or a peptide comprising a plurality of Ala and Gly residues. In some cases, the peptide is from 2 to 20 amino acids in length, optionally from 2 to 15, 2 to 10, 2 to 8, 2 to 6, 5 to 20, 5 to 15, 5 to 10, 10 to 20, 12 to 20, or 12 to 15 amino acids in length. In some cases, the linker comprises (Gly₄Ser)ₙ in which n is an integer selected from 1 to 10, e.g., 1, 2, 3, 4, 5, 6, 7, 8, 9, or 10.

In some embodiments, the first molecule that modulates the activity of a LIGHT receptor comprises a polypeptide that binds to LIGHT, HVEM, or LTβR. In some instances, the first molecule comprises a LIGHT polypeptide. In some instances, the LIGHT polypeptide comprises a mammalian LIGHT polypeptide, e.g., a LIGHT polypeptide from a rodent, a non-human primate, or a human. In some cases, the LIGHT polypeptide comprises a full-length polypeptide. In other cases, the LIGHT polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the LIGHT polypeptide is an inhibitor of LIGHT.

A non-limiting representative example of human LIGHT (p30 polypeptide) sequence (SEQ ID NO:4; the amino acid residues of the extracellular domain are underlined) comprises a polypeptide as set forth below:

MEESVVRPSVFVVDGQTDIPFTRLGRSHRRQSCSVARVGLGLLLLLMGA
GLAVQGWFLLQLHWRLGEMVTRLPDGPAGSWEQLIQERRSHEVNPAAHL
TGANSSLTGSGGPLLWETQLGLAFLRGLSYHDGALVVTKAGYYYIYSKV
QLGGVGCPLGLASTITHGLYKRTPRYPEELELLVSQQSPCGRATSSSRV
WWDSSFLGGVVHLEAGEEVVVRVLDERLVRLRDGTRSYFGAFMV,
or an equivalent thereof.

In some embodiments, the first molecule that modulates the activity of a LIGHT receptor comprises a polypeptide that binds to LIGHT. In some instances, the polypeptide that binds to LIGHT comprises a HVEM polypeptide or a LTβR polypeptide. In some instances, the HVEM polypeptide comprises a mammalian HVEM polypeptide, e.g., a HVEM polypeptide from a rodent, a non-human primate, or a human (e.g., SEQ ID NO: 1). In some cases, the HVEM polypeptide comprises a full-length polypeptide. In other cases, the HVEM polypeptide comprises a functional fragment, for example, comprising a truncation of about 5, 10, 15, 20, 25, 30, 35, 40, 50, or more amino acid residues from the N-terminus, the C-terminus, or a combination thereof. In some instances, the LTβR polypeptide comprises a mammalian LTβR polypeptide, e.g., a LTβR polypeptide from a rodent, a non-human primate, or a human (e.g., SEQ ID NO: 2). In some cases, the LTβR polypeptide comprises a full-length polypeptide. In other cases, the LTβR polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the HVEM polypeptide and/or the LTβR polypeptide is an inhibitor of LIGHT.

In some embodiments, the first molecule that modulates the activity of a LIGHT receptor comprises a peptidomimetic that modulates LIGHT, HVEM, or LTβR. In some cases, the first molecule is an inhibitor of LIGHT. As used herein, the term “mimetic” refers to a synthetic chemical compound which has substantially the same structural and/or functional characteristics as the reference molecule. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy activity.

In some embodiments, the first molecule that modulates the activity of a LIGHT receptor comprises a small molecule that modulates LIGHT. As described herein, the small molecule can include those that can bind selectively as well as those that bind non-selectively to LIGHT, HVEM, or LTβR in solution, in solid phase, in vitro, ex vivo or in vivo. The term “selective” can refer to a small molecule modulator (e.g., inhibitor) that binds specifically to the target entity (e.g., LIGHT, HVEM, or LTβR) and does not significantly bind to a non-ligand or non-target entity. A non-selective modulator (e.g., inhibitor) means that the modulator (e.g., the inhibitor) is not selective for the entity to which it binds, i.e., it cross-reacts with other entities. Exemplary small molecule modulators of LIGHT can further include salts or derivatives of a small molecule modulator.

In some embodiments, the first molecule that modulates the activity of a LIGHT receptor comprises an antibody that binds to LIGHT, an antibody that binds to HVEM, or an antibody that binds to LTβR. Exemplary LIGHT antibodies include, for example, antibodies that bind to human LIGHT, a non-human primate LIGHT, a rodent LIGHT, or a combination thereof. Non-limiting examples of antibodies that bind to human LIGHT include clone T5-39 (BioLegend, San Diego, Calif.), clone 115520 (R&D Systems, Minneapolis, Minn.), clones A-20 and C-20 (Santa Cruz Biotech, Santa Cruz, Calif.), and clone 4E3 (Novus Biologicals, Inc., Littleton, Colo.). Exemplary antibodies that bind to human HVEM include MAB3561 (R&D Systems), R718 (BD Biosciences), and HVEM Antibody D-5 (Santa Cruz Biotechnology). Exemplary antibodies that bind to human LTβR include MAB6291 and AF629 (R&D Systems), and 31G4D8 (BioLegend®).

TNF-like Ligand 1A (TL1A) (also known as tumor necrosis factor superfamily member 15 or TNFSF15) is a member of the TNF superfamily. TL1A can be expressed by multiple cell types including endothelial cells and fibroblasts and is upregulated by stimulation with proinflammatory cytokines such as TNF-α and IL-1. TL1A expression has also been detected on antigen-presenting cells and lymphocytes such as dendritic cells (DCs), macrophages, and T cells. TL1A can promote inflammation through acting on cells such as T cells and innate lymphoid cells or non-lymphoid cells via the tumor necrosis factor receptor superfamily (TNFRSF) cell surface receptor death receptor 3 (DR3, also known as tumor necrosis factor receptor superfamily member 25, TNFRSF25, APO-3, TRAMP, LARD, and WSL-1). Both TL1A and LIGHT can also bind the soluble molecule decoy receptor 3 (DcR3 also known as tumor necrosis factor receptor superfamily member 6B, TNFRSF6B, TR6, and M68). DcR3 is a member of the tumor necrosis factor receptor superfamily (TNFRSF) and is thought to be a naturally produced inhibitor of TL1A and LIGHT.

As used herein, a second molecule that modulates the activity of a TL1A receptor refers to a molecule that alters the activity of the TL1A receptor. In some instances, the second molecule decreases, reduces, or limits the activity of the TL1A receptor, e.g., decreases the activity by about 10%, 20%, 30%, 40%, 50%, 60%, 70%, 80, 90%, or 100%. In some instances, the second molecule decreases the activity of the TL1A receptor by about 1-fold, 2-fold, 3-fold, 4-fold, 5-fold, 6-fold, 7-fold, 8-fold, 9-fold, 10-fold, 20-fold, 30-fold, 40-fold, 50-fold, 100-fold, 200-fold, 500-fold, or more. The second molecule can modulate the activity of the TL1A receptor either directly or indirectly, by binding to TL1A, or to its binding partner death receptor 3 (DR3). Modulators of the TL1A receptor include, but are not limited to, a fusion of an immunoglobulin with DR3 or DcR3; a polypeptide that binds to TL1A or DR3; a peptidomimetic that modulates TL1A or DR3; a small molecule that modulates TL1A or DR3; an antibody that binds to TL1A, or an antibody that binds to DR3.

In some embodiments, the second molecule that modulates the activity of the TL1A receptor is an inhibitor of TL1A. As used herein, the term “an inhibitor of TL1A” refers to a molecule that directly or indirectly inhibits or blocks binding of TL1A to DR3. Inhibitors therefore include molecules that bind to TL1A as well as molecules that bind to a TL1A receptor, e.g., DR3. TL1A inhibitors therefore include molecules that bind to TL1A and molecules that bind to DR3. In some cases, inhibitors of TL1A include, but are not limited to, a fusion of an immunoglobulin with DR3 or DcR3; a polypeptide that binds to TL1A or DR3; a peptidomimetic that modulates TL1A or DR3; a small molecule that modulates TL1A or DR3; an antibody that binds to TL1A, or an antibody that binds to DR3.

In some embodiments, the second molecule that modulates the activity of a TL1A receptor comprises a fusion of an immunoglobulin with a DR3 polypeptide or a DcR3 polypeptide. In some instances, the DR3 polypeptide comprises a mammalian DR3 polypeptide, e.g., a DR3 polypeptide from a rodent, a non-human primate, or a human. In some cases, the DR3 polypeptide comprises a full-length polypeptide. In other cases, the DR3 polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the fusion polypeptide comprising an immunoglobulin and a DR3 polypeptide is an inhibitor of TL1A.

A non-limiting representative example of human DR3 sequence comprises a polypeptide as set forth below (SEQ ID NO: 5)

MEQRPRGCAAVAAALLLVLLGARAQGGTRSPRCDCAGDFHKKIGLFCCR
GCPAGHYLKAPCTEPCGNSTCLVCPQDTFLAWENHHNSECARCQACDEQ
ASQVALENCSAVADTRCGCKPGWFVECQVSQCVSSSPFYCQPCLDCGAL
HRHTRLLCSRRDTDCGTCLPGFYEHGDGCVSCPTSTLGSCPERCAAVCG
WRQMFWVQVLLAGLVVPLLLGATLTYTYRHCWPHKPLVTADEAGMEALT
PPPATHLSPLDSAHTLLAPPDSSEKICTVQLVGNSWTPGYPETQEALCP
QVTWSWDQLPSRALGPAAAPTLSPESPAGSPAMMLQPGPQLYDVMDAVP
ARRWKEFVRTLGLREAEIEAVEVEIGRFRDQQYEMLKRWRQQQPAGLGA
VYAALERMGLDGCVEDLRSRLQRGP,
or an equivalent thereof.

In some embodiments, the second molecule that modulates the activity of a TL1A receptor comprises a fusion of an immunoglobulin with a DcR3 polypeptide. In some instances, the DcR3 polypeptide comprises a mammalian DcR3 polypeptide, e.g., a DcR3 polypeptide from a rodent, a non-human primate, or a human. In some cases, the DcR3 polypeptide comprises a full-length polypeptide. In other cases, the DcR3 polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the fusion polypeptide comprising an immunoglobulin and a DcR3 polypeptide is an inhibitor of TL1A.

A non-limiting representative example of human DcR3 sequence comprises a polypeptide as set forth below (SEQ ID NO: 6)

MRALEGPGLSLLCLVLALPALLPVPAVRGVAETPTYPWRDAETGERLVC
AQCPPGTFVQRPCRRDSPTTCGPCPPRHYTQFWNYLERCRYCNVLCGER
EEEARACHATHNRACRCRTGFFAHAGFCLEHASCPPGAGVIAPGTPSQN
TQCQPCPPGTFSASSSSSEQCQPHRNCTALGLALNVPGSSSHDTLCTSC
TGFPLSTRVPGAEECERAVIDFVAFQDISIKRLQRLLQALEAPEGWGPT
PRAGRAALQLKLRRRLTELLGAQDGALLVRLLQALRVARMPGLERSVRE
RFLPVH,
or an equivalent thereof.

In some embodiments, the fusion polypeptide further comprises a linker that bridges the immunoglobulin with either DR3 or DcR3. In some instances, the linker comprises a peptide, e.g., a poly-Ala peptide, a poly-Gly peptide, or a peptide comprising a plurality of Ala and Gly residues. In some cases, the peptide is from 2 to 20 amino acids in length, optionally from 2 to 15, 2 to 10, 2 to 8, 2 to 6, 5 to 20, 5 to 15, 5 to 10, 10 to 20, 12 to 20, or 12 to 15 amino acids in length. In some cases, the linker comprises (Gly₄Ser)ₙ in which n is an integer selected from 1 to 10, e.g., 1, 2, 3, 4, 5, 6, 7, 8, 9, or 10.

In some embodiments, the second molecule that modulates the activity of a TL1A receptor comprises a polypeptide that binds to TL1A or DR3. In some instances, the second molecule comprises a TL1A polypeptide. In some instances, the TL1A polypeptide comprises a mammalian TL1A polypeptide, e.g., a TL1A polypeptide from a rodent, a non-human primate, or a human. In some cases, the TL1A polypeptide comprises a full-length polypeptide. In other cases, the TL1A polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the TL1A polypeptide is an inhibitor of TL1A.

A non-limiting representative example of human TL1A sequence comprises a polypeptide as set forth below (SEQ ID NO: 7)

MAEDLGLSFGETASVEMLPEHGSCRPKARSSSARWALTCCLVLLPFLAG
LTTYLLVSQLRAQGEACVQFQALKGQEFAPSHQQVYAPLRADGDKPRAH
LTVVRQTPTQHFKNQFPALHWEHELGLAFTKNRMNYTNKFLLIPESGDY
FIYSQVTFRGMTSECSEIRQAGRPNKPDSITVVITKVTDSYPEPTQLLM
GTKSVCEVGSNWFQPIYLGAMFSLQEGDKLMVNVSDISLVDYTKEDKTF
FGAFLL,
or an equivalent thereof.

In some embodiments, the second molecule that modulates the activity of a TL1A receptor comprises a polypeptide that binds to TL1A. In some instances, the polypeptide that binds to TL1A comprises a DR3 polypeptide or a DcR3 polypeptide. In some instances, the DR3 polypeptide comprises a mammalian DR3 polypeptide, e.g., a DR3 polypeptide from a rodent, a non-human primate, or a human (e.g., SEQ ID NO: 5). In some cases, the DR3 polypeptide comprises a full-length polypeptide. In other cases, the DR3 polypeptide comprises a functional fragment, for example, comprising a truncation of about 5, 10, 15, 20, 25, 30, 35, 40, 50, or more amino acid residues from the N-terminus, the C-terminus, or a combination thereof. In some instances, the DcR3 polypeptide comprises a mammalian DcR3 polypeptide, e.g., a DcR3 polypeptide from a rodent, a non-human primate, or a human (e.g., SEQ ID NO: 6). In some cases, the DcR3 polypeptide comprises a full-length polypeptide. In other cases, the DcR3 polypeptide comprises a functional fragment, for example, comprising a truncation of about 5, 10, 15, 20, 25, 30, 35, 40, 50, or more amino acid residues from the N-terminus, the C-terminus, or a combination thereof. In some cases, the DR3 polypeptide and/or the DcR3 polypeptide is an inhibitor of TL1A.

In some embodiments, the second molecule that modulates the activity of a TL1A receptor comprises a peptidomimetic that modulates TL1A or DR3. In some cases, the second molecule is an inhibitor of TL1A or DR3. As used herein, the term “mimetic” refers to a synthetic chemical compound which has substantially the same structural and/or functional characteristics as the reference molecule. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy activity.

In some embodiments, the second molecule that modulates the activity of a TL1A receptor comprises a small molecule that modulates TL1A or DR3. As described herein, the small molecule can include those that can bind selectively as well as those that bind non-selectively to TL1A or DR3 in solution, in solid phase, in vitro, ex vivo or in vivo. The term “selective” can refer to a small molecule modulator (e.g., inhibitor) that binds specifically to the target entity (e.g., TL1A or DR3) and does not significantly bind to a non-ligand or non-target entity. A non-selective modulator (e.g., inhibitor) means that the modulator (e.g., the inhibitor) is not selective for the entity to which it binds, i.e., it cross-reacts with other entities. Exemplary small molecule modulators of TL1A or DR3 can further include salts or derivatives of a small molecule modulator.

In some embodiments, the second molecule that modulates the activity of a TL1A receptor comprises an antibody that binds to TL1A or an antibody that binds to DR3. Exemplary TL1A antibodies include, for example, antibodies that bind to human TL1A, non-human primate TL1A, a rodent TL1A, or a combination thereof. Non-limiting examples of antibodies that bind to human TL1A include AF744 and MAB7441 (R&D Systems), L4G9 (Enzo Life Sciences, Inc.), and Ab85566 (Abcam). Exemplary antibodies that bind to human DR3 include MAB943 (R&D Systems), LS-B7731 (LifeSpan BioSciences), and anti-DR3 antibody from Biorbyt.

As used herein, antibodies include mammalian, human, humanized, humaneered or primatized forms of heavy or light chain, V_{H} and V_{L}, respectively, immunoglobulin (Ig) molecules. An “antibody” means any monoclonal or polyclonal immunoglobulin molecule, such as IgM, IgG, IgA, IgE, IgD, and any subclass thereof, which includes intact immunoglobulin molecules, two full length heavy chains linked by disulfide bonds to two full length light variable domains, V_{H} and V_{L}, individually or in any combination, as well as subsequences, such as Fab, Fab′, (Fab′)₂, Fv, Fd, scFv and sdFv, unless otherwise expressly stated.

An antibody that binds to LIGHT, HVEM, LTβR, TL1A, or DR3 means that the antibody has an affinity for LIGHT, HVEM, LTβR, TL1A, or DR3. “Binding” or “binds” is where the binding is selective between the two referenced molecules. Thus, binding of an antibody for LIGHT, HVEM, LTβR, TL1A, or DR3 is that which is selective for an epitope present in LIGHT, HVEM, LTβR, TL1A, or DR3. In some instances, the binding is a specific binding, which can be distinguished from non-specific when the dissociation constant (K_{D}) is less than about 1×10⁻⁵M or less than about 1×10⁻⁶M or 1×10⁻⁷ M. Selective binding can be distinguished from non-selective binding using assays known in the art (e.g., immunoprecipitation, ELISA, Western blotting) with appropriate controls.

In some embodiments, the antibody is a full-length antibody or an antigen binding fragment thereof. The antibody fragment can be a Fab, Fab′, F(ab′)₂, Fv, Fd, single-chain Fv (scFv), trispecific (Fab₃), bispecific (Fab₂), diabody ((V_{L}-V_{H})₂ or (V_{H}-V_{L})₂), triabody (trivalent), tetrabody (tetravalent), minibody ((scFv-CH)₂), bispecific single-chain Fv (Bis-scFv), IgGdeltaCH2, scFv-Fc, or (scFv)₂-Fc. In some cases, the antibody is a humanized antibody, a human antibody, a chimeric antibody, a murine antibody, a monoclonal antibody, or a polyclonal antibody.

In some instances, the antibody is a multispecific antibody that binds to LIGHT, HVEM, or LTβR and one or more of TL1A or DR3. As used herein, a multispecific antibody refers to an antibody that binds to two or more target epitopes. In some instances, the multispecific antibody comprises a first antigen binding domain that binds to LIGHT, HVEM, or LTβR, a second antigen binding domain that binds to TL1A or DR3, and optionally a third antigen domain that 1) binds to an additional target epitope selected from LIGHT, HVEM, LTβR, TL1A, or DR3 that is not the same as the first antigen binding domain or the second antigen binding domain, 2) binds to the Fcγ receptor of an antibody to modulate effector functions; or 3) an alternative target that is associated with a fibrotic disease, skin disease or inflammation, autoimmune disorder, or a respiratory disease. In some instances, the multispecific antibody comprises a first antigen binding domain that binds to LIGHT, HVEM, or LTβR and a second antigen binding domain that binds to TL1A. In some instances, the multispecific antibody comprises a first antigen binding domain that binds to LIGHT, HVEM, or LTβR and a second antigen binding domain that binds to DR3. In some instances, the multispecific antibody comprises a first antigen binding domain that binds to LIGHT and a second antigen binding domain that binds to TL1A or DR3. In some instances, the multispecific antibody comprises a first antigen binding domain that binds to HVEM and a second antigen binding domain that binds to TL1A or DR3. In some instances, the multispecific antibody comprises a first antigen binding domain that binds to LTβR and a second antigen binding domain that binds to TL1A or DR3. In some instances, the multispecific antibody comprises a first antigen binding domain that binds to LIGHT and a second antigen binding domain that binds to TL1A. In some instances, the multispecific antibody comprises a first antigen binding domain that binds to LIGHT and a second antigen binding domain that binds to DR3. In some instances, the multispecific antibody comprises a first antigen binding domain that binds to HVEM and a second antigen binding domain that binds to TL1A. In some instances, the multispecific antibody comprises a first antigen binding domain that binds to HVEM and a second antigen binding domain that binds to DR3. In some instances, the multispecific antibody comprises a first antigen binding domain that binds to LTβR and a second antigen binding domain that binds to TL1A. In some instances, the multispecific antibody comprises a first antigen binding domain that binds to LTβR and a second antigen binding domain that binds to DR3. In some cases, the multispecific antibody further comprises a third antigen domain that 1) binds to an additional target epitope selected from LIGHT, HVEM, LTβR, TL1A, or DR3 that is not the same as the first antigen binding domain or the second antigen binding domain, 2) binds to the Fcγ receptor of an antibody to modulate effector functions; or 3) an alternative target that is associated with a fibrotic disease, skin disease or inflammation, autoimmune disorder, or a respiratory disease.

In some embodiments, the multispecific antibody is a bispecific antibody that binds to LIGHT, HVEM, or LTβR and one or more of TL1A or DR3. Exemplary bispecific antibody formats include, but are not limited to, scFv, diabody, triabody, tetrabody, minibody, Bis-scFv, and tetravalent bispecific antibodies (e.g., fusion of an IgG antibody with a single chain domain). Additional bispecific antibody formats can be found in Labrijn, et al., “Bispecific antibodies: a mechanistic review of the pipeline,” Nature Reviews 18: 585-608 (2019).

In some embodiments, the bispecific antibody binds to LIGHT, HVEM, or LTβR and one or more of TL1A or DR3. In some instances, the bispecific antibody comprises a first antigen binding domain that binds to LIGHT, HVEM, or LTβR and a second antigen binding domain that binds to TL1A. In some instances, the bispecific antibody comprises a first antigen binding domain that binds to LIGHT, HVEM, or LTβR and a second antigen binding domain that binds to DR3. In some instances, the bispecific antibody comprises a first antigen binding domain that binds to LIGHT and a second antigen binding domain that binds to TL1A or DR3. In some instances, the bispecific antibody comprises a first antigen binding domain that binds to HVEM and a second antigen binding domain that binds to TL1A or DR3. In some instances, the bispecific antibody comprises a first antigen binding domain that binds to LTβR and a second antigen binding domain that binds to TL1A or DR3. In some instances, the bispecific antibody comprises a first antigen binding domain that binds to LIGHT and a second antigen binding domain that binds to TL1A. In some instances, the bispecific antibody comprises a first antigen binding domain that binds to LIGHT and a second antigen binding domain that binds to DR3. In some instances, the bispecific antibody comprises a first antigen binding domain that binds to HVEM and a second antigen binding domain that binds to TL1A. In some instances, the bispecific antibody comprises a first antigen binding domain that binds to HVEM and a second antigen binding domain that binds to DR3. In some instances, the bispecific antibody comprises a first antigen binding domain that binds to LTβR and a second antigen binding domain that binds to TL1A. In some instances, the bispecific antibody comprises a first antigen binding domain that binds to LTβR and a second antigen binding domain that binds to DR3.

A “human antibody” means that the amino acid sequence of the antibody is fully human, i.e., human heavy and light chain variable and constant regions. The antibody amino acids are coded for in the human DNA antibody sequences or exist in a human antibody. Fully human antibodies can be made by human antibody transgenic or transchromosomic animals, such as mice, or by isolation from human antibody producing cell lines (e.g., B cells) by recombinant DNA methodology known to the skilled artisan, such as gene cloning by reverse transcriptase polymerase chain reaction (RT-PCR). An antibody that is non-human may be made fully human by substituting non-human amino acid residues with amino acid residues that exist in a human antibody. Amino acid residues present in human antibodies, CDR region maps and human antibody consensus residues are known in the art (see, e.g., Kabat, Sequences of Proteins of Immunological Interest, 4^{th} Ed. US Department of Health and Human Services. Public Health Service (1987); Chothia and Lesk, J. Mol. Biol. (1987) 186:651; Padlan Mol. Immunol. (1994) 31:169; and Padlan Mol. Immunol. (1991) 28:489). Methods of producing human antibodies are also described, for example, in WO 02/43478 and WO 02/092812.

The term “humanized,” when used in reference to an antibody, means that the antibody sequence has non-human amino acid residues of one or more complementarity determining regions (CDRs) that specifically bind to the antigen in an acceptor human immunoglobulin molecule, and one or more human amino acid residues in the framework region (FR) that flank the CDRs. Any mouse, rat, guinea pig, goat, non-human primate (e.g., ape, chimpanzee, macaque, orangutan, etc.) or other animal antibody may be used as a CDR donor for producing humanized antibody. Human framework region residues can be replaced with corresponding non-human residues (e.g., from the donor variable region). Residues in the human framework regions can therefore be substituted with a corresponding residue from the non-human CDR donor antibody. A humanized antibody may include residues, which are found neither in the human antibody nor in the donor CDR or framework sequences. The use of antibody components derived from humanized monoclonal antibodies reduces problems associated with the immunogenicity of non-human regions. Methods of producing humanized antibodies are known in the art (see, for example, U.S. Pat. Nos. 5,225,539; 5,530,101, 5,565,332 and 5,585,089; Riechmann et al., (1988) Nature 332:323; EP 239,400; WO91/09967; EP 592,106; EP 519,596; Padlan Molecular Immunol. (1991) 28:489; Studnicka et al., Protein Engineering (1994) 7:805; Singer et al., J. Immunol. (1993) 150:2844; and Roguska et al., Proc. Nat'l. Acad. Sci. USA (1994) 91:969).

The term “humanized,” when used in reference to an antibody, means that the antibody sequence has high affinity for antigen but has a greater number of human germline sequences than a humanized antibody. Typically humaneered antibody has at least 90% or more human germline sequences.

The term “chimeric antibody” refers to an antibody in which different portions are derived from different animal species, such as those having a variable region derived from a murine monoclonal antibody and a human immunoglobulin constant region, e.g., humanized antibodies. In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used.

Monoclonal antibodies are made by methods known in the art (Kohler et al., Nature, 256:495(1975); and Harlow and Lane, Using Antibodies: A Laboratory Manual, Cold Spring Harbor Laboratory, 1999). Briefly, monoclonal antibodies can be obtained by injecting mice with antigen. The polypeptide or peptide used to immunize an animal may be derived from translated DNA or chemically synthesized and conjugated to a carrier protein. Commonly used carriers which are chemically coupled to the immunizing peptide include, for example, keyhole limpet hemocyanin (KLH), thyroglobulin, bovine serum albumin (BSA), and tetanus toxoid. Antibody production is verified by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from hybridoma cultures. Monoclonal antibodies can be isolated and purified from hybridoma cultures by a variety of established techniques which include, for example, affinity chromatography with Protein-A Sepharose, size-exclusion chromatography, and ion-exchange chromatography (see e.g., Coligan et al., Current Protocols in Immunology sections 2.7.1-2.7.12 and sections 2.9.1-2.9.3; and Barnes et al., “Methods in Molecular Biology,” 10:79-104, Humana Press (1992)).

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt- cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

It is to be inferred without explicit recitation and unless otherwise intended, that when the present disclosure relates to a polypeptide, protein, polynucleotide, an equivalent or a biologically equivalent of such is intended within the scope of this disclosure. As used herein, the term “biological equivalent thereof” is intended to be synonymous with “equivalent thereof” when referring to a reference protein, polypeptide or nucleic acid, intends those having minimal homology while still maintaining desired structure or functionality. Unless specifically recited herein, it is contemplated that any of the above also includes equivalents thereof. For example, an equivalent intends at least about 70% homology or identity, or at least 80% homology or identity and alternatively, or at least about 85%, or alternatively at least about 90%, or alternatively at least about 95%, or alternatively at least 98% percent homology or identity and/or exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The phrase “equivalent polypeptide” or “equivalent peptide fragment” refers to protein, polynucleotide, or peptide fragment encoded by a polynucleotide that hybridizes to a polynucleotide encoding the exemplified polypeptide or its complement of the polynucleotide encoding the exemplified polypeptide, under high stringency and/or which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology or identity. Percentage homology or identity can be determined by sequence comparison using programs such as BLAST run under appropriate conditions. In one aspect, the program is run under default parameters.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding the chimeric PVX described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical,” percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. Polypeptides include full length native polypeptide, and “modified” forms such as subsequences, variant sequences, fusion/chimeric sequences and dominant-negative sequences. As used herein the term “amino acid” refers to either natural and/or unnatural or synthetic amino acids, including glycine and both the D and L optical isomers, amino acid analogs and peptidomimetics.

Peptides include L- and D-isomers, and combinations thereof. Peptides can include modifications typically associated with post-translational processing of proteins, for example, cyclization (e.g., disulfide or amide bond), phosphorylation, glycosylation, carboxylation, ubiquitination, myristylation, or lipidation. Modified peptides can have one or more amino acid residues substituted with another residue, added to the sequence or deleted from the sequence. Specific examples include one or more amino acid substitutions, additions or deletions (e.g., 1-3, 3-5, 5-10, 10-20, or more).

Subsequences and fragments refer to polypeptides having one or more fewer amino acids in comparison to a reference (e.g., native) polypeptide sequence. An antibody subsequence that specifically binds to LIGHT, HVEM or LTβR can retain at least a part of its binding or LIGHT inhibitory or antagonist activity.

A variant peptide can have a sequence with 50%, 60%, 70%, 75%, 80%, 85%, 90%, 95%, 96%, 97%, 98%, or more identity to a reference sequence. Variant sequences include naturally occurring alterations of sequence, due to intra-species polymorphisms or different species, as well as artificially produced alterations of sequence. Sequence homology between species is in the range of about 70-80%. An amino acid substitution is one example of a variant.

A “conservative substitution” is the replacement of one amino acid by a biologically, chemically or structurally similar residue. Biologically similar means that the substitution is compatible with an activity or function of the unsubstituted sequence. Structurally similar means that the amino acids have side chains with similar length, such as alanine, glycine and serine, or having similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

Peptides synthesized and expressed as fusion proteins have one or more additional domains linked thereto, and are also referred to as chimeric polypeptides. The additional domain(s) may confer an additional function upon the sequence. For example, HVEM-IgG or LTβR-IgG fusion proteins can have LIGHT inhibitory activity.

The term “fusion,” when used in reference to two or more molecules (e.g., polypeptides) means that the molecules are covalently attached. A particular example for attachment of two protein sequences is an amide bond or equivalent. The term “chimeric,” and grammatical variations thereof, when used in reference to a protein, means that the protein is comprised of one or more heterologous amino acid residues from one or more different proteins.

The term “heterologous,” when used in reference to a polypeptide, means that the polypeptide is not normally contiguous with the other polypeptide in its natural environment. Thus, a chimeric polypeptide means that a portion of the polypeptide does not exist fused with the other polypeptide in normal cells. In other words, a chimeric polypeptide is a molecule that does not normally exist in nature, i.e., such a molecule is produced by the hand of man, e.g., artificially produced through recombinant DNA technology.

As described supra, peptide mimetics can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., induce or stabilize a secondary structure, e.g., a beta turn, gamma turn, beta sheet, alpha helix conformation, and the like. For example, a polypeptide can be characterized as a mimetic when one or more of the residues are joined by chemical means other than an amide bond. Individual peptidomimetic residues can be joined by amide bonds, non-natural and non-amide chemical bonds other chemical bonds or coupling means including, for example, glutaraldehyde, N-hydroxysuccinimide esters, bifunctional maleimides, N,N′-dicyclohexylcarbodiimide (DCC) or N,N′-diisopropylcarbodiimide (DIC). Linking groups alternative to the amide bond include, for example, ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide and Backbone Modifications,” Marcel Decker, NY).

Peptides and peptidomimetics can be produced and isolated using a variety of methods known in the art. Full length peptides and fragments (subsequences) can be synthesized using chemical methods known in the art (see, e.g., Caruthers, Nucleic Acids Res. Symp. Ser. (1980) 215; Horn, Nucleic Acids Res. Symp. Ser. (1980) 225; and Banga, A. K., Therapeutic Peptides and Proteins, Formulation, Processing and Delivery Systems (1995) Technomic Publishing Co., Lancaster, Pa.). Peptide synthesis can be performed using various solid-phase techniques (see, e.g., Roberge, Science (1995) 269:202; Merrifield, Methods Enzymol. (1997) 289:3). Automated synthesis may be achieved, e.g., using a peptide synthesizer.

Individual synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Peptides and peptide mimetics can also be synthesized using combinatorial methodologies. Techniques for generating peptide and peptidomimetic libraries are known, and include, for example, multipin, tea bag, and split-couple-mix techniques (see, for example, al-Obeidi, Mol. Biotechnol. (1998) 9:205; Hruby, Curr. Opin. Chem. Biol. (1997) 1:114; Ostergaard, Mol. Divers. (1997) 3:17; and Ostresh, Methods Enzymol. (1996) 267:220). Modified peptides can be further produced by chemical modification methods (see, e.g., Belousov, Nucleic Acids Res. (1997) 25:3440; Frenkel, Free Radic. Biol. Med. (1995) 19:373; and Blommers, Biochemistry (1994) 33:7886).

In some embodiments, a first molecule that modulates the activity of a LIGHT receptor and a second molecule that modulates the activity of a TL1A receptor can each include those that can bind selectively as well as those that bind non-selectively to a ligand or target (e.g., LIGHT, HVEM, LTβR, TL1A, or DR3) in solution, in solid phase, in vitro, ex vivo or in vivo. As used herein, the term “selective” when used in reference to a LIGHT modulator (e.g., a LIGHT inhibitor) means that the modulator (e.g., inhibitor) binds specifically to the target entity (e.g., LIGHT, HVEM, or LTβR) and does not significantly bind to a non-ligand or non-target entity. As used herein, the term “selective” when used in reference to a TL1A modulator (e.g., a TL1A inhibitor) means that the modulator (e.g., inhibitor) binds specifically to the target entity (e.g., TL1A or DR3) and does not significantly bind to a non-ligand or non-target entity. A non-selective modulator (e.g., inhibitor) means that the modulator (e.g., the inhibitor) is not selective for the entity to which it binds, i.e., it cross-reacts with other entities.

LIGHT and/or TL1A modulators (e.g., LIGHT and/or TL1A inhibitors) include variants and derivatives that retain at least a part or all of an activity of the non-variant or non-derivatized modulator (e.g., non-variant or non-derivatized inhibitor). A particular activity (e.g., antagonist or inhibitory activity) of a LIGHT or TL1A modulator (e.g., LIGHT or TL1A inhibitor) may be less than or greater than the activity of a corresponding non-variant or non-derivatized LIGHT or TL1A modulator (e.g., LIGHT or TL1A inhibitor). For example, a LIGHT or TL1A modulator (e.g., LIGHT or TL1A inhibitor) variant or derivative may have less or greater activity than non-variant or non-derivatized LIGHT or TL1A modulator (e.g., LIGHT or TL1A inhibitor).

Non-limiting examples of activities that can be retained, at least in part, include inhibitory or antagonist activity, binding affinity (e.g., K_{d}), avidity and binding selectivity (specificity) or non-selectivity. The variant or derivatized modulator (e.g., inhibitor) can exhibit an activity (e.g., binding affinity) that is greater or less than a corresponding non-variant or non-derivatized modulator (e.g., inhibitor), e.g., greater or less inhibitory activity, binding affinity (e.g., K_{d}), avidity or binding selectivity (specificity) or non-selectivity. For example, “at least a part” of an activity of a modulator (e.g., an inhibitor) can be when the variant or derivatized agent has less of an inhibitory activity, e.g., 10-25%, 25-50%, 50-60%, 60-70%, 70-75%, 75-80%, 80-85%, 85-90%, 90-95%, 95-99%, 100%, or any percent or numerical value or range or value within such ranges. An activity of a modulator (e.g., an inhibitor) can be when the variant or derivatized agent has more inhibitory activity, e.g., 110-125%, 125-150%, 150-175%, 175-200%, 200-250%, 250-300%, 300-400%, 400-500%, 500-1000%, 1000-2000%, 2000-5000%, or more, or any percent or numerical value or range or value within such ranges. At least a part of binding affinity of a modulator (e.g., an inhibitor) can be when the variant or derivatized inhibitor has less affinity, e.g., 1-3-fold, 1-5-fold, 2-5 fold, 5-10-fold, 5-15-fold, 10-15-fold, 15-20-fold, 20-25-fold, 25-30-fold, 30-50-fold, 50-100 fold, 100-500-fold 500-1000-fold, 1000-5000-fold, or less (e.g., K_{d}), or any numerical value or range of values within such ranges. At least a part of binding affinity of a modulator (e.g., an inhibitor) can be when the variant or derivatized inhibitor has more affinity, e.g., 1-3-fold, 1-5-fold, 2-5 fold, 5-10-fold, 5-15-fold, 10-15-fold, 15-20-fold, 20-25-fold, 25-30-fold, 30-50-fold, 50-100 fold, 100-500-fold 500-1000-fold, 1000-5000-fold, or more (e.g., K_{d}), or any numerical value or range of values within such ranges.

Dissociation (K_{d}) constants can be measured using radiolabeled inhibitors in competitive binding assays with increasing amounts of unlabelled inhibitor to generate saturation curves. The target, ligand or receptor used in the binding assay (e.g., LIGHT, HVEM, LTβR, TL1A, DR3, or DcR3) can be expressed in vitro, on cells or be present in extracts. Association (Kₐ) and dissociation (K_{d}) constants can be measured using surface plasmon resonance (SPR) (Rich and Myszka, Curr. Opin. Biotechnol. 11:54 (2000); Englebienne, Analyst.123:1599 (1998)). SPR methods for real time detection and monitoring of protein binding rates are known and are commercially available and can be used to determine dissociation (K_{d}) constants (BiaCore 2000, Biacore AB, Upsala, Sweden; and Malmqvist, Biochem. Soc. Trans. 27:335 (1999)).

LIGHT and/or TL1A modulators (e.g., LIGHT and/or TL1A inhibitors) can be identified by assays known in the art. For example, the amount of activity can be assessed directly, such as measuring the particular activity (e.g., inhibitor activity, binding affinity, avidity, selectivity (specificity) or non-selectivity). For example, a LIGHT modulator (e.g., a LIGHT inhibitor) and/or a TL1A modulator (e.g., a TL1A inhibitor) can be identified by inhibition of HVEM or LTβR mediated lymphocyte activation or cell proliferation or by inhibition of inflammation by DR3. A LIGHT modulator (e.g., a LIGHT inhibitor) and/or a TL1A modulator (e.g., a TL1A inhibitor) can also be identified by change in cell expression of a marker, such as ICAM expression. LIGHT modulators (e.g., LIGHT inhibitors) and/or TL1A modulators (e.g., TL1A inhibitors) can further be identified by the ability to inhibit binding of purified LIGHT to purified HVEM or LTβR (or HVEM-IgG or LTβR-IgG fusion proteins) or by the ability to inhibit binding of TL1A to purified DR3 or DcR3 (or DR3-IgG or DcR3-IgG fusion proteins), for example, when immobilized on a substrate (e.g., plastic) by ELISA, or when any of the molecules are transfected into cells that can be identified by labeling with the corresponding binding partner by flow cytometry. More particularly, for ELISA assays, plate bound LIGHT or TL1A can be pre-incubated with LIGHT or TL1A specific inhibitory molecules and blockade of receptor fusion protein binding measured by detection of the binding of the Fc fusion protein or lack of binding. Blockade of cell surface associated LIGHT or TL1A binding to receptors is assessed by pre-incubation of LIGHT or TL1A inhibitory molecules with cell lines expressing LIGHT or TL1A on the surface followed by addition of receptor Fc fusion proteins. Assessment of inhibition is measured by detection of binding of the receptor fusion proteins or lack of binding by flow cytometry. Inhibition of LIGHT signaling or TL1A signaling in vitro can be determined by inhibiting LIGHT or TL1A mediated chemokine secretion from colonic epithelial cells (HT29).

In some embodiments, disclosed herein is a method of reducing or inhibiting a fibrotic disease in a subject in need thereof, which comprises, consists essentially of, or yet further consists of modulating the activity of LIGHT (p30 polypeptide) and the activity of TNF-like Ligand 1A (TL1A) in the subject in need thereof. In some instances, the method comprises, consists essentially of, or yet further consists of administering to the subject a sufficient amount of at least one of a first molecule that modulates the activity of LIGHT (p30 polypeptide) and a second molecule that modulates the activity of TNF-like Ligand 1A (TL1A), to reduce or inhibit a fibrotic disease in the subject.

In some embodiments, disclosed herein is a method of treating a skin disease or inflammation in a subject in need thereof, which comprises, consists essentially of, or yet further consists of modulating the activity of LIGHT (p30 polypeptide) and the activity of TNF-like Ligand 1A (TL1A) in the subject in need thereof. In some instances, the method comprises, consists essentially of, or yet further consists of administering to the subject a sufficient amount of at least one of a first molecule that modulates the activity of LIGHT (p30 polypeptide) and a second molecule that modulates the activity of TNF-like Ligand 1A (TL1A), to treat the skin disease or inflammation in the subject.

In some embodiments, disclosed herein is a method of treating an autoimmune disorder in a subject in need thereof, which comprises, consists essentially of, or yet further consists of modulating the activity of LIGHT (p30 polypeptide) and the activity of TNF-like Ligand 1A (TL1A) in the subject in need thereof. In some instances, the method comprises, consists essentially of, or yet further consists of administering to the subject a sufficient amount of at least one of a first molecule that modulates the activity of LIGHT (p30 polypeptide) and a second molecule that modulates the activity of TNF-like Ligand 1A (TL1A), to treat the autoimmune disorder in the subject.

In some embodiments, disclosed herein is a method of treating a respiratory disease in a subject in need thereof, which comprises, consists essentially of, or yet further consists of modulating the activity of LIGHT (p30 polypeptide) and the activity of TNF-like Ligand 1A (TL1A) in the subject in need thereof. In some instances, the method comprises, consists essentially of, or yet further consists of administering to the subject a sufficient amount of at least one of a first molecule that modulates the activity of LIGHT (p30 polypeptide) and a second molecule that modulates the activity of TNF-like Ligand 1A (TL1A), to treat the respiratory disease in the subject.

In some embodiments, disclosed herein is a method of reducing or inhibiting the activity of a LIGHT (p30 polypeptide) receptor and/or the activity of a TNF-like Ligand 1A (TL1A) receptor in a subject in need thereof, which comprises, consists essentially of, or yet further consists of modulating the activity of LIGHT (p30 polypeptide) and the activity of TNF-like Ligand 1A (TL1A) in the subject in need thereof. In some instances, the method comprises, consists essentially of, or yet further consists of administering to the subject a sufficient amount of at least one of a first molecule that modulates the activity of LIGHT (p30 polypeptide) and a second molecule that modulates the activity of TNF-like Ligand 1A (TL1A), to reduce or inhibits the activity of a LIGHT (p30 polypeptide) receptor and/or the activity of a TNF-like Ligand 1A (TL1A) receptor in the subject.

In some instances, modulating the activity of LIGHT and the activity of TL1A in the subject in need thereof comprises reducing, decreasing, suppressing, limiting, controlling, or inhibiting the activity of LIGHT, and reducing, decreasing, suppressing, limiting, controlling, or inhibiting the activity of TL1A.

In some embodiments, a fibrotic disease comprises fibrosis (or fibrotic scarring) of parenchymal organs and tissues. In some instances, a fibrotic disease comprises fibrosis of the lung, liver, skin, kidney, brain, heart, joints, intestine, or the bone marrow, or a combination thereof. In some instances, the fibrotic disease comprises fibrosis of the lung, liver, or skin. In some instances, the fibrotic disease comprises interstitial lung disease (ILD), liver cirrhosis, or idiopathic pulmonary fibrosis.

In some cases, the fibrotic disease is a skin fibrotic disease or disorder. As used herein, a “skin fibrotic disease or disorder” means a condition, disorder or disease related to a skin or dermal tissue. Exemplary skin fibrotic disease or disorder include, but are not limited to, scleroderma and atopic dermatitis.

In some embodiments, a skin disease or inflammation comprises atopic dermatitis, scleroderma, psoriasis, onchocercal dermatitis, nephrogenic fibrosing dermopathy, mixed connective tissue disease, scleromyxedema, keloid, sclerodactyly, or eosinophilic fasciitis. In some cases, the skin disease or inflammation comprises atopic dermatitis. In some cases, the skin disease or inflammation comprises scleroderma. In some cases, the skin disease or inflammation comprises psoriasis.

In some embodiments, a respiratory disease comprises asthma, allergic asthma, bronchiolitis, pleuritis, chronic obstructive pulmonary disease (COPD), extrinsic bronchial asthma, allergic rhinitis, eosophageal allergy, or gastrointestinal allergy. In some cases, the respiratory disease comprises nodules, eosinophilia, rheumatism, dermatitis and swelling (NERDS). In some cases, the respiratory disease comprises airway obstruction, apnea, asbestosis, atelectasis, berylliosis, bronchiectasis, bronchiolitis, bronchiolitis obliterans organizing pneumonia, bronchitis, bronchopulmonarydysplasia, empyema, pleural empyema, pleural epiglottitis, hemoptysis, hypertension, kartagener syndrome, meconium aspiration, pleural effusion, pleurisy, pneumonia, pneumothorax, respiratory distress syndrome, respiratory hypersensitivity, respiratory tract infections, rhinoscleroma, scimitar syndrome, severe acute respiratory syndrome, silicosis, or tracheal stenosis.

Skin inflammation, skin fibrosis, scleroderma, or a respiratory disease include allergic and non-allergic skin inflammation, skin fibrosis, scleroderma, or a skin fibrotic disease or disorder, which may be provoked by a variety of factors including aberrant or undesirable immune responses. Alternatively or in addition to, skin inflammation, skin fibrosis, scleroderma, or a skin fibrotic disease or disorder may be caused by or associated with irritant particles (allergens such as pollen, dust, venoms, cotton, dander, foods). Skin inflammation, skin fibrosis, scleroderma, or a skin fibrotic diseases or disorders can be acute, chronic, mild, moderate or severe.

An “allergen” is a substance that can promote, stimulate or induce skin inflammation, skin fibrosis, scleroderma, atopic dermatitis or skin fibrotic diseases or disorders in a subject. Allergens include plant/tree pollens, insect venoms, animal dander, house dust mite, dust, fungal spores, latex, food and drugs (e.g., penicillin). Examples of particular allergens include proteins specific to the following genera: Canis (Canis familiaris); Dermatophagoides (e.g., Dermatophagoides farinae); Felis (Felis domesticus); Ambrosia (Ambrosia artemiisfolia); Lolium (e.g., Lolium perenne or Lolium multiflorum); Cryptomeria (Cryptomeria japonica); Alternaria(Alternaria alternata); Alder; Alnus (Alnus gultinosa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis or Parietaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis(e.g., Dactylis glomerata); Festuca(e.g., Festuca elatior); Poa (e.g., Poa pratensisor Poa compressa); Avena (e.g., Avena sativa); Holcus (e.g., Holcus lanatus); Anthoxanthum(e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis). Allergens also include peptides and polypeptides used in experimental animal models of allergy and asthma, including ovalbumin (OVA) and Schistosoma mansoni egg antigen.

In some embodiments, the autoimmune disorder comprises systemic sclerosis (e.g., scleroderma), rheumatoid arthritis (RA), lupus (e.g., systemic lupus erythematosus or SLE), inflammatory bowel disease (IBD), eosinophilic esophagitis (EoE), ankylosing spondylitis (AS), experimental autoimmune encephalomyelitis (EAE), or an autoimmune inflammatory disease of the central nervous system (CNS). In some cases, the autoimmune disorder comprises systemic sclerosis (scleroderma).

In accordance with the invention, provided herein are methods of reducing or decreasing progression, severity, frequency, duration, susceptibility or probability of a fibrotic disease, a skin disease or inflammation, an autoimmune disorder, or a respiratory disease. In one embodiment, a method includes administering to a subject an amount of LIGHT modulator (e.g., LIGHT inhibitor) and/or TL1A modulator (e.g., TL1A inhibitor) sufficient to reduce or decrease progression, severity, frequency, duration, susceptibility or probability of one or more adverse symptoms associated with a fibrotic disease, a skin disease or inflammation, an autoimmune disorder, or a respiratory disease.

In some instances, compositions such as LIGHT modulators (e.g., LIGHT inhibitors) and/or TL1A modulators (e.g., TL1A inhibitors) can be administered in sufficient or effective amounts to achieve a therapeutic benefit in a subject treated in accordance with the invention. An “amount sufficient” or “amount effective” includes an amount that, in a given subject, can have a desired outcome or effect. The “amount sufficient” or “amount effective” can be an amount of a LIGHT modulator (e.g., a LIGHT inhibitor) and/or a TL1A modulator (e.g., a TL1A inhibitor) that provides, in single or multiple doses, alone or in combination with one or more other (second) compounds or agents (e.g., a drug), treatments or therapeutic regimens, a long or short term detectable response, a desired outcome or beneficial effect in a particular given subject of any measurable or detectable degree or duration (e.g., for minutes, hours, days, months, years, or cured).

An amount sufficient or an amount effective can but need not be provided in a single administration and can but need not be administered alone (i.e., without a second drug, agent, treatment or therapeutic regimen), or in combination with another compound, agent, treatment or therapeutic regimen. In addition, an amount sufficient or an amount effective need not be sufficient or effective if given in single or multiple doses without a second compound, agent, treatment or therapeutic regimen, since additional doses, amounts or duration above and beyond such doses, or additional drugs, agents, treatment or therapeutic regimens may be included in order to be effective or sufficient in a given subject. Further, an amount sufficient or an amount effective need not be effective in each and every subject, nor a majority of subjects in a given group or population. Thus, as some subjects may not benefit from such treatments an amount sufficient or an amount effective means sufficiency or effectiveness in a particular subject, not a group or the general population. As is typical for such methods, some subjects will exhibit a greater or less response to a method of the invention, including treatment/therapy.

Reducing, inhibiting decreasing, eliminating, delaying, halting or preventing a progression or worsening or an adverse symptom of the condition, disorder or disease is a satisfactory outcome. The dose amount, frequency or duration may be proportionally increased or reduced, as indicated by the status of the condition, disorder or disease being treated, or any adverse side effects of the treatment or therapy. Dose amounts, frequencies or duration also considered sufficient and effective are those that result in a reduction of the use of another drug, agent, treatment or therapeutic regimen or protocol. For example, a LIGHT modulator (e.g., a LIGHT inhibitor) and/or a TL1A modulator (e.g., a TL1A inhibitor) is considered as having a beneficial or therapeutic effect if contact, administration or delivery in vivo results in the use of a lesser amount, frequency or duration of another drug, agent, treatment or therapeutic regimen or protocol to treat the condition, disorder or disease, or an adverse symptom thereof.

An “amount sufficient” or “amount effective” includes reducing, preventing, delaying or inhibiting onset, reducing, inhibiting, delaying, preventing or halting the progression or worsening of, reducing, relieving, alleviating the severity, frequency, duration, susceptibility or probability of one or more adverse or undesirable symptoms associated with the condition, disorder or disease of the subject. In addition, hastening a subject's recovery from one or more adverse or undesirable symptoms associated with the condition, disorder or disease is considered to be an amount sufficient or effective. Various beneficial effects and indicia of therapeutic benefit are as set forth herein and are known to the skilled artisan.

An “amount sufficient” or “amount effective,” in the appropriate context, can refer to therapeutic or prophylactic amounts. Therapeutically or prophylactically sufficient or effective amounts mean an amount that, in a given subject, detectably improves the condition, disorder or disease, such as an inflammatory condition, disorder or disease, as assessed by one or more objective or subjective clinical endpoints appropriate for the condition, disorder or disease.

Sufficiency or effectiveness of a particular treatment can be ascertained by various clinical indicia and endpoints. An “amount sufficient” or “amount effective” is therefore an amount that provides an objective or subjective reduction or improvement in progression, severity, frequency, susceptibility or probability of fibrosis, skin inflammation, skin fibrosis, an autoimmune disorder, a respiratory disease. Thus, a reduction, decrease, inhibition, delay, halt, prevention or elimination of one or more adverse symptoms of fibrosis, skin inflammation, skin fibrosis, an autoimmune disorder, a respiratory disease can be used as a measure of sufficiency or effectiveness.

An “amount sufficient” or “amount effective” also includes an amount that, when used in combination with another binding agent, drug, or treatment or therapeutic regimen, reduces the dosage frequency, dosage amount, or an adverse symptom or side effect of the other binding agent, drug or treatment or therapeutic regimen, or eliminates the need for the other binding agent, drug or treatment or therapeutic regimen. For example, an “amount sufficient” or “amount effective” of a LIGHT modulator (e.g., a LIGHT inhibitor) or a TL1A modulator (e.g., a TL1A inhibitor) could result in a reduction in the dosage frequency or dosage amount of a steroid, antihistamine, beta adrenergic agonist, anticholinergic, methylxanthine, anti-IgE, anti-leukotriene, anti-beta2 integrin, anti-CCR3 antagonist, or anti-selectin required to achieve the same clinical endpoint.

The terms “treat,” “therapy” and grammatical variations thereof when used in reference to a method means the method provides an objective or subjective (perceived) improvement in a subjects' condition, disorder or disease, or an adverse symptom associated with the condition, disorder or disease. Non-limiting examples of an improvement can therefore reduce or decrease the probability, susceptibility or likelihood that the subject so treated will manifest one or more symptoms of the condition, disorder or disease. Additional symptoms and physiological or psychological responses caused by or associated with fibrotic disease, skin disease, skin inflammation, skin fibrosis, an autoimmune disorder, or a respiratory disease are set forth herein and known in the art and, therefore, improvements in these and other adverse symptoms or physiological or psychological responses can also be included in the methods of the invention.

Methods of the invention therefore include providing a detectable or measurable beneficial effect or therapeutic benefit to a subject, or any objective or subjective transient or temporary, or longer-term improvement (e.g., cure) in the condition. Thus, a satisfactory clinical endpoint is achieved when there is an incremental improvement in the subject's condition or a partial reduction in the severity, frequency, duration or progression of one or more associated adverse symptoms or complications or inhibition, reduction, elimination, prevention or reversal of one or more of the physiological, biochemical or cellular manifestations or characteristics of the condition, disorder or disease. A therapeutic benefit or improvement (“ameliorate” is used synonymously) therefore need not be complete ablation of any or all adverse symptoms or complications associated with the condition, disorder or disease but is any measurable or detectable objectively or subjectively meaningful improvement in the condition, disorder or disease. For example, inhibiting a worsening or progression of the condition, disorder or disease, or an associated symptom (e.g., slowing or stabilizing one or more symptoms, complications or physiological or psychological effects or responses), even if only for a few days, weeks or months, even if complete ablation of the condition, disorder or disease, or an associated adverse symptom is not achieved is considered to be beneficial effect.

Prophylactic methods are included. “Prophylaxis” and grammatical variations thereof mean a method in accordance with the invention in which contact, administration or in vivo delivery to a subject is prior to manifestation or onset of a condition, disorder or disease (or an associated symptom or physiological or psychological response), such that it can eliminate, prevent, inhibit, decrease or reduce the probability, susceptibility, onset or frequency of having a condition, disorder or disease, or an associated symptom. Target subjects for prophylaxis can be one of increased risk (probability or susceptibility) of contracting the condition, disorder or disease, or an associated symptom, or recurrence of a previously diagnosed condition, disorder or disease, or an associated symptom, as set forth herein.

The term “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human.

In some embodiments, a method disclosed herein further comprises administering to the subject an additional therapeutic agent. Non-limiting examples of functional classes of compounds and agents useful as an additional therapeutic agent include anti-inflammatory, anti-allergy, and/or immunosuppressant drugs. Additional non-limiting examples of compounds and agents useful for employing in the invention, for example to treat a fibrotic disease, a skin disease, a skin inflammation, skin fibrosis, an autoimmune disease, or a respiratory disease include hormones, such as steroids (e.g., glucocorticoids); antihistamines; beta adrenergic agonists; anticholinergics; methylxanthines; anti-IgE; anti-leukotrienes; anti-beta2 integrins; anti-alpha-4 integrins; H1-receptor antagonists; anti-CCR3 antagonists; and anti-selectins.

Specific non-limiting examples of glucocorticoids include dexamethasone, triamcinolone acetonide (AZMACORT®), beclomethasone, dipropionate (VANCERIL®), flunisolide (AEROBID®), fluticasone propionate (FLOVENT®), prednisone, methylprednisolone and mometasonefuroate (ASMANEX®, TWISTHALER®). Specific non-limiting examples of antihistamines include chlorcyclizine, chlorpheniramine, triprolidine (ACTIFED®), diphenhydramine hydrochloride (BENADRYL®), fexofenadine hydrochloride (ALLEGRA®), hydroxyzine hydrochloride (ATARAX®), loratadine (CLARITIN®), promethazine hydrochloride (PHENERGAN®), pyrilamine; and anti-IgE omalizumab (XOLAIR®). Specific non-limiting example of beta adrenergic agonists include albuterol (VENTOLIN®; PROVENTIL®), Xopenex®, (S)-isomer subtracted from racemic albuterol (Sepracor Inc.), pirbuterol, epinephrine, racepinephrine, adrenaline, isoproterenol, salmeterol (Serevent®), metaproterenol (ALUPENT®), bitolterol (Tornalate®), fenoterol (BEROTEC®), formoterol (Foradil®), isoetharine, procaterol, β2-adrenoceptor and terbutaline (BRETHINE®, LAMISIL®). A specific non-limiting example of an anticholinergic (cholinergic receptor antagonist) includes ipratropium bromide (ATROVENT®) and tiotropium. Specific non-limiting examples of methylxanthines include theophylline, aminophylline, theobromine, cromolyn (Intal®) and nedocromil (Fisons). A specific non-limiting example of an anti-IgE is omalizumab (XOLAIR®). Specific non-limiting examples of anti-leukotrienes (leukotriene inhibitors) include cysteinyl-leukotriene (Cys-LT), Singulair® and Accolate®.

Anti-inflammatory agents useful for employing in the methods include cytokines and chemokines. Particular non-limiting examples of cytokines include anti-inflammatory cytokines such as IL-4 and IL-10. Anti-cytokines and anti-chemokines, such as antibodies that bind to pro-inflammatory cytokines, TNFα, IFNγ, IL-1, IL-2, IL-6, etc., as well as anti-Th2 cytokines such as IL-5, IL-13, etc., can be employed in the methods.

Immunosuppressant agents useful for employing in the methods described herein include, but are not limited to, corticosteroids; Janus kinase inhibitors such as tofacitinib, calcineurin inhibitors such as cyclosporine and tacrolimus; mTOR inhibitors such as sirolimus and everolimus; IMDH inhibitors such as azathioprine, leflunomide, and mycophenolate; and biologics such as abatacept, adalimumab, anakinra, certolizumab, etanercept, golimumab, infliximab, ixekizumab, natalizumab, rituximab, secukinumab, tocilizumab, ustekinumab, and vedolizumab.

Additional functional classes of compounds and agents useful as an additional therapeutic agent include selective or non-selective potassium channel activators (bronchodilatators); muscarinic M3 receptor antagonists; M2 receptor agonists; opioid receptor agonists (inhibit release of sensory neuropeptides); H3-receptor agonists (inhibit acetylcholine release); phospholipase A2 inhibitors; 5-lipoxygenase inhibitors; 5-lipoxygenase activating protein (FLAP) inhibitors; phosphodiesterase inhibitors; immunomodulating agents (Ciclosporine); antibody against adhesion molecules; and antagonists of tachykinins (e.g., Substance P or neurokinin).

In some instances, the first molecule and/or the second molecule and optionally the additional therapeutic agent are administered simultaneously.

In some instances, the first molecule and/or the second molecule and optionally the additional therapeutic agent are administered sequentially. In some cases, the first molecule and/or the second molecule are administered prior to administering the additional therapeutic agent. In some cases, the first molecule and/or the second molecule are administered after administering the additional therapeutic agent.

In some instances, the first molecule and/or the second molecule and/or the additional therapeutic agent are administered systemically.

In some instances, the first molecule and/or the second molecule and/or the additional therapeutic agent are administered locally.

In some cases, the first molecule and/or the second molecule and/or the additional therapeutic agent are administered by parenteral administration.

In some cases, the first molecule and/or the second molecule and/or the additional therapeutic agent are administered intravenously or subcutaneously.

In some embodiments, disclosed herein is a combination which comprises a first molecule that modulates the activity of LIGHT (p30 polypeptide) and a second molecule that modulates the activity of TNF-like Ligand 1A (TL1A). In some instances, the first molecule comprises a fusion of an immunoglobulin and a) herpesvirus entry mediator (HVEM) or b) lymphotoxin beta receptor (LTβR) polypeptide. In some cases, the first molecule comprises a polypeptide that binds to LIGHT, HVEM, or LTβR, a peptidomimetic that modulates LIGHT, or a small molecule that modulates LIGHT. In some cases, the first molecule comprises an antibody that binds to LIGHT, an antibody that binds to HVEM, or an antibody that binds to LTβR.

In some instances, the second molecule comprises a fusion of DR3 with an immunoglobulin, a fusion of DcR3 with an immunoglobulin, a polypeptide that binds to DR3, a peptidomimetic that modulates TL1A or DR3, or a small molecule that modulates TL1A or DR3. In some cases, the second molecule comprises an antibody that binds to TL1A or an antibody that binds to DR3.

In some embodiments, the antibody that binds to LIGHT, HVEM, or LTβR is a multispecific antibody that binds to LIGHT, HVEM, or LTβR and one or more of TL1A or DR3. In some instances, the multispecific antibody comprises a first antigen binding domain that binds to LIGHT, HVEM, or LTβR and a second antigen binding domain that binds to TL1A. In some instances, the multispecific antibody comprises a first antigen binding domain that binds to LIGHT, HVEM, or LTβR and a second antigen binding domain that binds to DR3. In some instances, the multispecific antibody comprises a first antigen binding domain that binds to LIGHT and a second antigen binding domain that binds to TL1A or DR3. In some instances, the multispecific antibody comprises a first antigen binding domain that binds to HVEM and a second antigen binding domain that binds to TL1A or DR3. In some instances, the multispecific antibody comprises a first antigen binding domain that binds to LTβR and a second antigen binding domain that binds to TL1A or DR3. In some instances, the multispecific antibody comprises a first antigen binding domain that binds to LIGHT and a second antigen binding domain that binds to TL1A. In some instances, the multispecific antibody comprises a first antigen binding domain that binds to LIGHT and a second antigen binding domain that binds to DR3. In some instances, the multispecific antibody comprises a first antigen binding domain that binds to HVEM and a second antigen binding domain that binds to TL1A. In some instances, the multispecific antibody comprises a first antigen binding domain that binds to HVEM and a second antigen binding domain that binds to DR3. In some instances, the multispecific antibody comprises a first antigen binding domain that binds to LTβR and a second antigen binding domain that binds to TL1A. In some instances, the multispecific antibody comprises a first antigen binding domain that binds to LTβR and a second antigen binding domain that binds to DR3. In some cases, the multispecific antibody comprises a bispecific antibody.

In some embodiments, the combination comprises a pharmaceutical composition comprising the first molecule that modulates the activity of LIGHT (p30 polypeptide), the second molecule that modulates the activity of TNF-like Ligand 1A (TL1A), and a pharmaceutically acceptable excipient.

Compositions including LIGHT modulators (e.g., LIGHT inhibitors) and TL1A modulators (e.g., TL1A inhibitors) can be included in a pharmaceutically acceptable carrier (excipient, diluent, vehicle or filling agent) for administration to a subject. The terms “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary active compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of the active ingredient thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Exemplary antifungals include agents such as benzoic acid, undecylenicalkanolamide, ciclopiroxolamine, polyenes, imidazoles, allylamine, thicarbamates, amphotericin B, butylparaben, clindamycin, econaxole, amrolfine, butenafine, naftifine, terbinafine, ketoconazole, elubiol, econazole, econaxole, itraconazole, isoconazole, miconazole, sulconazole, clotrimazole, enilconazole, oxiconazole, tioconazole, terconazole, butoconazole, thiabendazole, voriconazole, saperconazole, sertaconazole, fenticonazole, posaconazole, bifonazole, fluconazole, flutrimazole, nystatin, pimaricin, amphotericin B, flucytosine, natamycin, tolnaftate, mafenide, dapsone, caspofungin, actofunicone, griseofulvin, potassium iodide, Gentian Violet, ciclopirox, ciclopiroxolamine, haloprogin, ketoconazole, undecylenate, silver sulfadiazine, undecylenic acid, undecylenicalkanolamide and Carbol-Fuchsin.

The pH can be adjusted by use or addition of pharmacologically acceptable acids or bases. Examples of inorganic acids include: hydrochloric acid, hydrobromic acid, nitric acid, sulfuric acid, and/or phosphoric acid. Examples of organic acids are: ascorbic acid, citric acid, malic acid, tartaric acid, maleic acid, succinic acid, fumaric acid, acetic acid, formic acid and/or propionic acid, etc. Acids which form an acid addition salt with the active ingredient may also be used. Examples of bases include alkali metal hydroxides and alkali metal carbonates. If such bases are used, the resulting salts which are contained in the pharmaceutical formulation, are typically compatible with the acid. If desired, mixtures of acids or bases may also be used.

Pharmaceutical compositions can optionally be formulated to be compatible with a particular route of administration. Thus, pharmaceutical compositions include carriers (excipients, diluents, vehicles or filling agents) suitable for administration by various routes and delivery to targets, topically, locally, regionally or systemically.

Exemplary routes of administration for contact or in vivo delivery which a composition can optionally be formulated include skin, dermis or epidermis, oral, buccal, intrapulmonary, intrauterine, intradermal, topical, dermal, parenteral, sublingual, subcutaneous, intravascular, intrathecal, intraarticular, intracavity, transdermal, iontophoretic, intraocular, ophthalmic, optical, intravenous, intramuscular, intraglandular, intraorgan, intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof.

Pharmaceutical compositions and delivery systems appropriate for compositions and methods of the invention are known to the skilled artisan (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315)

LIGHT modulators (e.g., LIGHT inhibitors), TL1A modulators (e.g., TL1A inhibitors) and pharmaceutical compositions thereof can be packaged in unit dosage form (capsules, troches, cachets, lozenges, or tablets) for ease of administration and uniformity of dosage. “Unit dosage form” as used herein refers to physically discrete units suited as dosages for treatment or therapy. Each unit contains a predetermined quantity of agent in association with the pharmaceutical carrier (excipient, diluent, vehicle or filling agent) which, when administered in one or more doses, is calculated to produce a desired beneficial effect. Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compositions for transdermal administration, such as “patches” adapted to remain in contact with the epidermis of the intended recipient for an extended or brief period of time. The individual unit dosage forms can be included in multi-dose kits or containers.

Dose amounts, frequency and duration for binding agents, including LIGHT inhibitors, or pro-drugs thereof, can be can be empirically determined in appropriate animal models. Dose amounts, frequency and duration can also be determined and optimized in human clinical trials.

The dosage amount can range from about 0.0001 mg/kg of subject body weight/day to about 1,000.0 mg/kg of subject body weight/day. Of course, doses can be more or less, as appropriate, for example, 0.00001 mg/kg of subject body weight to about 10,000.0 mg/kg of subject body weight, about 0.001 mg/kg, to about 1,000 mg/kg, about 0.01 mg/kg, to about 100 mg/kg, or about 0.1 mg/kg, to about 10 mg/kg of subject body weight over a given time period, e.g., 1, 2, 3, 4, 5 or more hours, days, weeks, months, years, in single bolus or in divided/metered doses.

As a non-limiting example, for treatment of a fibrotic disease, a skin disease, a skin inflammation, skin fibrosis, an autoimmune disease, or a respiratory disease, a subject may be administered in single bolus or in divided/metered doses in the range of about 10 to 50,000 micrograms (“mcg”)/day, 10 to 20,000 mcg/day, 10 to 10,000 mcg/day, 25-1,000 mcg/day, 25 to 400 mcg/day, 25-200 mcg/day, 25-100 mcg/day or 25-50 mcg/day, which can be adjusted to be greater or less according to the weight of the subject, e.g., per pound, kilogram, etc.

LIGHT modulators (e.g., LIGHT inhibitors), TL1A modulators (e.g., TL1A inhibitors), combinations of LIGHT and/or TL1A modulators (e.g., LIGHT and/or TL1A inhibitors) and other active ingredients and pharmaceutical formulations thereof can be administered to a subject at any frequency, as a single bolus or in divided/metered doses, one, two, three, four or more times over a given time period, e.g., per hour, day, week, month or year. Exemplary dosage frequencies for a fibrotic disease, a skin disease, a skin inflammation, skin fibrosis, an autoimmune disease, or a respiratory disease can vary, but are typically from 1-7 times, 1-5 times, 1-3 times, 2-times or once, daily, weekly or monthly, to reduce, inhibit, decrease, delay, prevent, halt or eliminate progression, severity, frequency, duration, or probability of one or more adverse symptoms of the conditions, disorders or diseases, as set forth herein or that would be apparent to one skilled in the art. Timing of contact, administration or in vivo delivery can be dictated by the condition, disorder or disease to be treated. For example, an amount can be administered to the subject substantially contemporaneously with, or within about 1-60 minutes or hours of the onset of a symptom associated with or caused by a fibrotic disease, a skin disease, a skin inflammation, skin fibrosis, an autoimmune disease, or a respiratory disease.

Dosage amount, frequency or duration can be increased, if necessary, or reduced, for example, once control of the condition, disorder or disease is achieved, dose amounts, frequency or duration can be reduced. Other conditions, disorders or diseases associated with fibrosis, with the skin, with an autoimmune disease, or with a respiratory disease can be similarly treated, dosing amount, frequency or duration reduced, when adequate control of the condition, disorder or disease is achieved.

Of course, the dosage amount, frequency and duration can vary depending upon the judgment of the skilled artisan which will consider various factors such as whether the treatment is prophylactic or therapeutic, the type or severity of the condition, disorder or disease, the associated symptom to be treated, the clinical endpoint(s) desired such as the type and duration of beneficial or therapeutic effect. Additional non-limiting factors to consider in determining appropriate dosage amounts, frequency, and duration include previous or simultaneous treatments, potential adverse systemic, regional or local side effects, the individual subject (e.g., general health, age, gender, race, bioavailability), condition of the subject such as other disorders or diseases present and other treatments or therapies that the subject has or is undergoing (e.g., medical history). The skilled artisan will appreciate the factors that may influence the dosage, frequency and duration required to provide an amount sufficient to provide a subject with a beneficial effect, such as a therapeutic benefit.

In some embodiments, the invention further provides kits including LIGHT modulators (e.g., LIGHT inhibitors) and TL1A modulators (e.g., TL1A inhibitors) suitable for practicing the methods, treatment protocols or therapeutic regimes herein, and suitable packing material. In one embodiment, a kit includes a LIGHT modulator (e.g., a LIGHT inhibitor), a TL1A modulator (e.g., a TL1A inhibitor), and instructions for administering or using the LIGHT or TL1A modulator (e.g., LIGHT or TL1A inhibitor). In another embodiment, a kit includes a LIGHT modulator (e.g., a LIGHT inhibitor), a TL1A modulator (e.g., a TL1A inhibitor), an article of manufacture for delivery of the LIGHT or TL1A modulator (e.g., LIGHT or TL1A inhibitor) to the target area, organ, tissue or system (e.g., skin) and instructions for administering the LIGHT or TL1A modulator (e.g., LIGHT or TL1A inhibitor).

The term “packing material” refers to a physical structure housing a component of the kit. The material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., floppy diskette, ZIP disk), optical disk such as CD- or DVD-ROM/RAM, DVD, 1V1P3, magnetic tape, or an electrical storage media such as RAM and ROM or hybrids of these such as magnetic/optical storage media, FLASH media or memory type cards.

Labels or inserts can include identifying information of one or more components therein (e.g., the binding agent or pharmaceutical composition), dose amounts, clinical pharmacology of the active agent(s) including mechanism of action, pharmacokinetics and pharmacodynamics. Labels or inserts can include information identifying manufacturer information, lot numbers, and location and date of manufacture.

Labels or inserts can include information on a condition, disorder or disease for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or therapeutic regimes described herein.

Labels or inserts can include information on any benefit that a component may provide, such as a therapeutic benefit. Labels or inserts can include information on potential adverse side effects, such as warnings to the subject or clinician regarding situations where it would not be appropriate to use a particular composition (e.g., a LIGHT modulator or a TL1A modulator). For example, adverse side effects are generally more likely to occur at higher dose amounts, frequency or duration of the active agent and, therefore, instructions could include recommendations against higher dose amounts, frequency or duration. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities. Non-limiting examples of adverse side effects include, for example, hypersensitivity, rash, neurological effects such as tachycardia; palpitations; headache; tremor and nervousness.

### EXAMPLES

These examples are provided for illustrative purposes only and not to limit the scope of the claims provided herein.

### Example 1

Diseases such as atopic dermatitis (AD) and psoriasis present with extensive tissue remodeling, features shared with diseases of the lungs, such as severe asthma. The inventors have shown that a genetic deficiency in the TNF superfamily molecule LIGHT (TNFSF14, CD258), and blocking LIGHT binding to its receptors, reduced remodeling in the lungs in mouse models of severe asthma and systemic sclerosis (1, 2). The inventors have further shown that another TNF family molecule, TL1A (TNFSF15), also contributes to remodeling in the same models of asthma and systemic sclerosis, and TL1A drives pathology independent of LIGHT, suggesting it plays a complementary and synergistic role to LIGHT (3). LIGHT interacts with the TNF superfamily receptors HVEM (herpesvirus entry mediator, TNFRSF14) and LTβR (lymphotoxin beta receptor, TNFRSF3), and TL1A with DR3 (TNFRSF25) (4-6). The studies disclosed herein investigate the combination use of LIGHT and TL1A to control skin tissue remodeling relevant to AD and psoriasis, directly driving deregulated activity in keratinocytes and dermal fibroblasts. In support, the inventors have reported that LIGHT-deficient mice were protected from developing skin inflammation in models of scleroderma (7) and allergen-induced AD (8). Moreover, subcutaneous injection of recombinant LIGHT into naïve mice induced features of AD, scleroderma, and psoriasis (8), and the inventors have found that LIGHT-deficient mice also display reduced epidermal reactions in a model of imiquimod-induced psoriasis. The inventors have observed that the absence of TL1A/DR3 interactions also markedly limits disease in the same models of AD and psoriasis, and that keratinocytes and fibroblasts co-express DR3 together with the receptors for LIGHT. The inventors determine that LIGHT and TL1A are effectors of skin pathology through driving epidermal hyperplasia and remodeling molecules such as collagen, periostin, and alpha smooth muscle actin in keratinocytes and/or fibroblasts. Through production of other factors such as TSLP, IL-33, and chemokines in these structural cells, LIGHT and TL1A maintain disease through orchestrating immune inflammatory cells in the skin.

Study 1: Defining the inflammatory activities of LIGHT and TL1A in keratinocytes. The inventors examine human epidermal keratinocytes in vitro to determine that LIGHT and TL1A synergistically control transcriptional signatures in these cells that are found in human AD and psoriasis skin lesions, including key inflammatory cytokines and chemokines, and that together they drive keratinocyte hyperplasia and disrupt barrier function. The inventors test with conditional deletion of LTβR, HVEM, and DR3 in keratinocytes to determine that direct activity of LIGHT and TL1A through these receptors is essential for pathology and immune activity in mouse models of AD and psoriasis. The results demonstrate how LIGHT and TL1A work together to transcriptionally regulate keratinocytes and that this correlates with transcriptomes associated with human AD or psoriasis. They show the extent of overlap and divergence in activity between these TNF family proteins and the three keratinocyte-acting inflammatory cytokines currently most recognized as being of relevance to AD and psoriasis pathogenesis, namely IL-13, TNF, and IL-17. They also show that LIGHT and TL1A induce common signaling pathways in keratinocytes, and connect functional activities of these proteins to intracellular activities that might be shared with other pro-inflammatory cytokines.

Study 2: Identifying the inflammatory activities of LIGHT and TL1A in dermal fibroblasts. The inventors determine LIGHT and TL1A co-operate to promote myofibroblast differentiation, upregulate production of collagen, periostin, and alpha smooth muscle actin, and promote expression of inflammatory cytokines and chemokines using human dermal fibroblasts relevant for AD or psoriasis. In vivo, the inventors determine the importance of signaling through HVEM, LTβR, and DR3, in fibroblasts with conditional deletion in mouse models of AD and psoriasis. The results demonstrate the combined inflammatory effects of LIGHT and TL1A in dermal fibroblasts and the relevance of LIGHT and TL1A transcriptional regulation in these cells to transcriptomes present in human AD or psoriasis skin lesions. They also show how LIGHT and TL1A differ from other key fibroblast-acting inflammatory cytokines (TNF, IL-17, IL-13), and reinforce the conclusions from Study 1 regarding the importance of common signaling pathways controlling cellular activity in AD or psoriasis.

Study 3: Determining if therapeutic targeting of LIGHT and TL1A can block or reverse progression of skin inflammatory disease. The inventors examine therapeutic blockade of LIGHT together with TL1A and its suppression of ongoing skin inflammatory symptoms in models of AD and psoriasis, and determine that skin tissue remodeling can be reversed. The inventors' preclinical findings support the notion of single or combination targeting of these proteins in clinical trials in AD or psoriasis.

Strategy

Keratinocytes and dermal fibroblasts are thought to be of central importance to skin inflammatory disorders, responding excessively to immune cell-derived cytokines and proliferating and/or producing fibrotic mediators such as collagen, and then expressing further pro-inflammatory cytokines and chemokines which attract and maintain cells of the innate and adaptive immune systems in the skin (9-11). Despite these apparent commonalities, each disorder has been suggested to develop through distinct immunological programs, with for example Th2/Tc2 immunity dominating atopic dermatitis (12) and TNF/Th1 and Th17/Th22 immunity dominating psoriasis (12-19). Current FDA-approved therapies targeting IL-4Rα, and TNF and IL-17/IL-23, in AD and psoriasis respectively, support this dichotomy. However, studies have suggested AD clinical subtypes also can display strong Th17/Th22 phenotypes with the Th2 response (20, 21). New RNA-seq data from patient biopsies (22), and the results on two TNF superfamily proteins, LIGHT and TL1A, described below, further suggest that the distinctions between the skin disorders might not be as profound as previously thought and indicate that there are some immunological driver molecules that work together that are shared between these diseases.

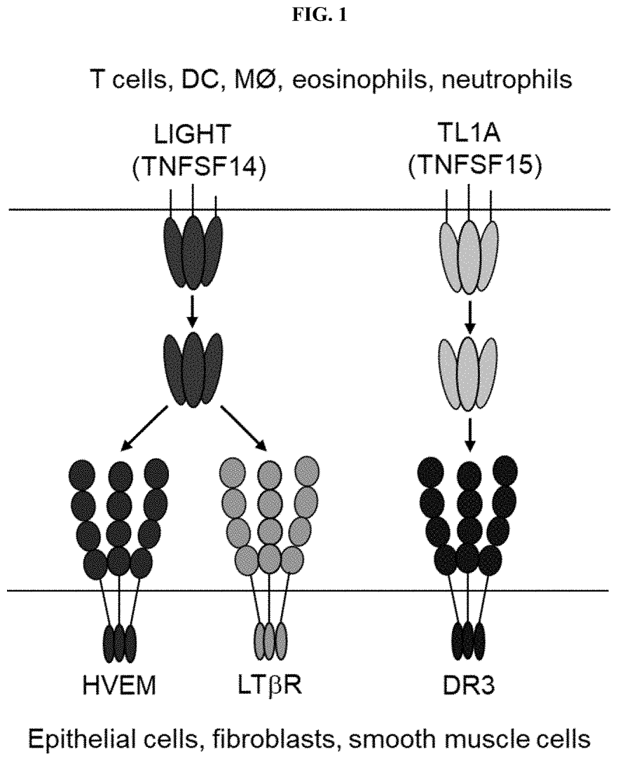

TNF family proteins are key modulators of several immune-mediated disorders (5, 6, 23, 24). LIGHT, aka TNFSF14 and CD258, is a soluble and membrane expressed pro-inflammatory molecule (25-28) that acts through two receptors (FIG. 1), the herpesvirus entry mediator (HVEM; TNFRSF14; CD270) and the lymphotoxin β receptor (LTβR; TNFRSF3) (29, 30). HVEM is found on most lymphoid and several non-lymphoid cells, and LTβR is on APC and non-lymphoid cells (5, 24, 30-32), as described below. LIGHT is a product of T cells (29, 30, 32), and is made by all CD4 T cells regardless of Th phenotype as well as by CD8 T cells. Moreover, LIGHT can be made by macrophages, neutrophils, and eosinophils (unpublished data). The inventors have reported, with gene-deficient animals, and blocking studies, that LIGHT is an orchestrator of lung tissue remodeling in models of severe asthma and systemic sclerosis, and importantly that recombinant LIGHT (rLIGHT) injected alone into the naïve mouse lungs could reproduce this fibrotic activity (1, 2). Lung remodeling has features in common with the skin inflammatory diseases. This includes epithelial cell involvement (hyperplasia); the activity of epithelial-derived cytokines such as TSLP, or cytokines that might come from T cells or other sources such as IL-13 or IL-17; and deposition of proteins such as collagen or periostin derived from epithelial cells or fibroblasts. The inventors then determined that LIGHT is relevant to skin inflammation. In support, in a model of scleroderma, LIGHT was induced in the skin, and the inventors have reported that LIGHT-deficient mice exhibited strongly reduced dermal and epidermal thickening (7). To extend this data, the inventors set up models of AD and psoriasis. The AD model is induced by epicutaneous exposure of the shaved back to house dust mite (HDM) allergen, given over 14 days (33-35). This drives a Th2 response resulting in scaling, thickening of the epidermal and dermal layers, and collagen deposition in the latter. The psoriasis model uses a cream containing the TLR7/8 agonist imiquimod, given on the shaved back for 7-9 days (36-39). This drives primarily a Th17 inflammatory response, also resulting in epidermal thickening, although dermal collagen deposition in this model is minor.

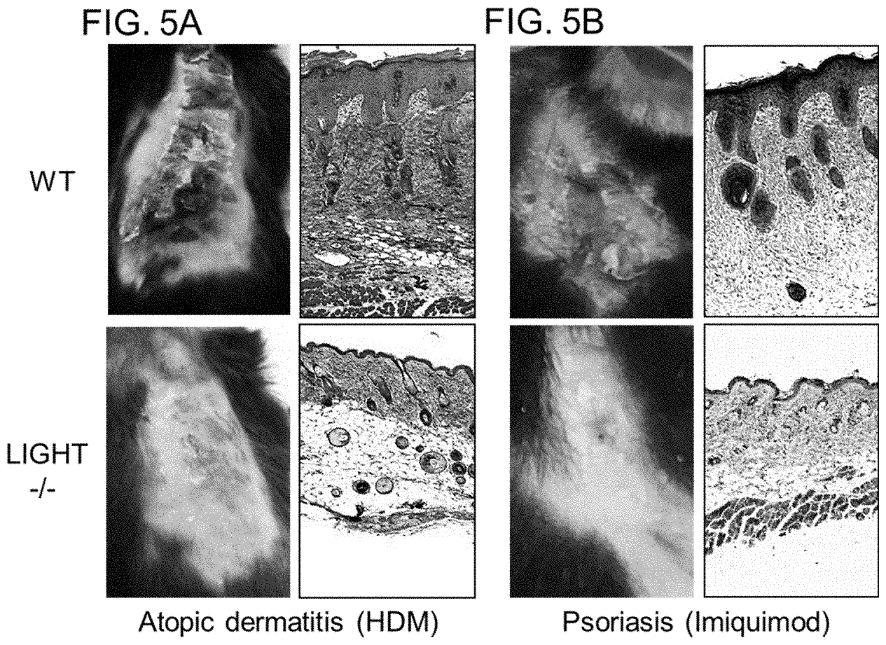

The inventors have shown that LIGHT-deficient mice were protected from developing atopic dermatitis (AD)-like skin lesions (8). This included reduced dermal and epidermal thickening, fewer infiltrating cells, and weak expression of IL-4, IL-5, IL-13, TGF-β, collagen, TSLP, and periostin (FIG. 5A and (8)). Moreover, similar result was observed in a psoriasis model with reduced epidermal thickening (FIG. 5B), fewer infiltrating cells, as well as reduced expression by mRNA of cytokine IL-17.

Given the potential overlap in activities of several TNF-like proteins (5, 31), the inventors initiated studies of another molecule in the superfamily, and obtained data that suggests that TL1A (TNF-like ligand 1A; TNFSF15) exerts similar, overlapping, and likely synergistic activities with LIGHT. TL1A is an inducible molecule, also soluble, and can be made by macrophages, dendritic cells, eosinophils, and neutrophils (FIG. 1, (5, 6, 24)). TL1A acts through the stimulatory receptor DR3 (TNFRSF25). DR3 is expressed on all T cells and upregulated after activation, and is inducible on ILC (40), and it is constitutive or inducible on several non-lymphoid cells as described below. The inventors tested if TL1A was required for lung tissue remodeling and fibrosis characteristic of SSc and severe asthma and found reduced features of these diseases in DR3 knockout mice and when TL1A was therapeutically blocked (3). The inventors further investigated if TL1A and DR3 might also be required for skin inflammatory disease, and found reduced dermal and/or epidermal activity in DR3-deficient mice in the models of AD and psoriasis.

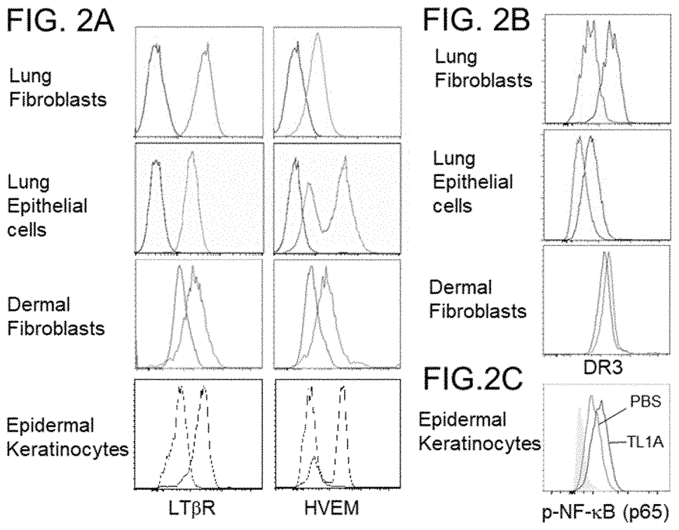

The current disclosure examines mechanistic and pre-clinical studies to understand the cell types in the skin that LIGHT and TL1A act upon and how they control the skin inflammatory response. Both LIGHT and TL1A directly regulate skin pathology by working together to drive excessive inflammatory activity in keratinocytes and dermal fibroblasts. The inventors have shown that both of LIGHT's receptors are expressed on human lung epithelial cells and lung fibroblasts (FIG. 2A), and showed that LIGHT promotes inflammatory mediators in these cells (2, 41, 42). In line with these cell types being a likely target of LIGHT in the skin, the inventors found HVEM and LTβR expressed on human (and mouse, not shown) keratinocytes and dermal fibroblasts ((FIG. 2A) and (7, 8)). DR3 has been visualized on human kidney tubular epithelial cells (43), mouse intestinal myofibroblasts (44), and human fibroblast-like synoviocytes (45). DR3 has also been shown to be expressed on human lung epithelial cells and fibroblasts, and found it can induce pro-inflammatory and remodeling-relevant activity in these cells (FIG. 2B; 3). The inventors show that similar cell types in the skin are targets of TL1A. Low-level expression of DR3 on human keratinocytes and dermal fibroblasts were observed (FIG. 2B) and recombinant TL1A (rTL1A) enhanced NF-κB activation in keratinocytes (FIG. 2C), providing support that TL1A and LIGHT function together on both cell types.

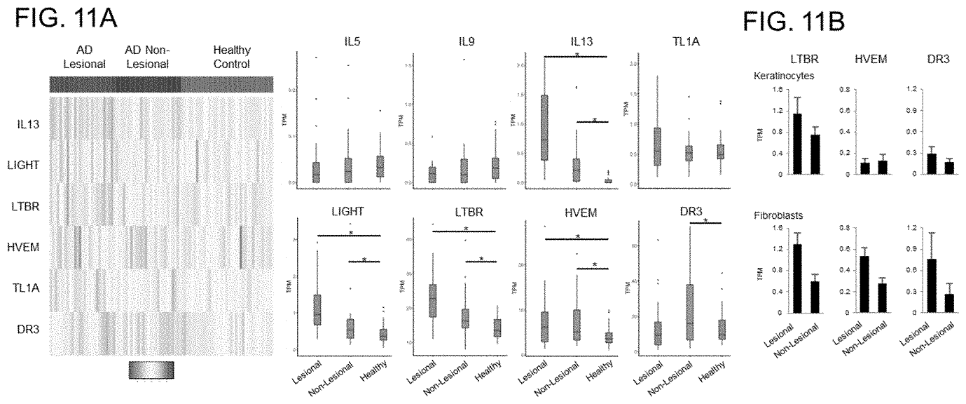

Studies of human samples have shown upregulation of LIGHT and/or TL1A molecules and their receptors in patients with AD and psoriasis. Soluble LIGHT was found elevated in the serum of AD patients, particularly those with severe disease (46, 47), and soluble HVEM, that can be cleaved from the cell surface after interacting with LIGHT, was increased in AD serum as well (47, 48). Serum soluble TL1A was also increased in psoriasis and AD (49, 50). Polymorphisms of decoy receptor 3, a soluble antagonist of both TL1A and LIGHT, also associate with susceptibility to AD (51, 52). As these SNPs likely lead to defective function of this decoy protein, the findings suggest there is enhanced activity of TL1A and LIGHT in AD regardless of whether levels of these cytokines are elevated. The inventors also analyzed published RNA-seq data (22, 53) to determine gene expression in AD skin biopsies. This showed expression of LIGHT, TL1A, HVEM, LTβR, and DR3 in lesional skin (FIG. 11A). Moreover, transcripts in both lesional and non-lesional skin were found for the three relevant receptors in the cells that are the focus of these studies, namely keratinocytes and fibroblasts (FIG. 11B).

Inflammatory Activities of LIGHT and TL1A in Keratinocytes

Rationale: HVEM, LTβR, and DR3 co-operatively drive epidermal thickening of relevance to AD and psoriasis by promoting keratinocyte hyperplasia (proliferation), through direct signaling as well as indirectly by promoting keratinocyte factors that act in an autocrine manner to increase proliferation. The inventors also find that these receptors maintain ongoing skin inflammation by increasing production of inflammatory factors from keratinocytes that keep infiltrating immune cells in the skin and/or drive their functional activities. Keratinocytes can be primary sources of cytokines like TSLP, IL-33, IL-23 and IL-19, and chemokines that attract cells such as macrophages, neutrophils, and eosinophils (68, 69). The inventors examine whether LIGHT and TL1A together promote these features with studies of human keratinocytes in vitro, and use targeted studies in vivo of HVEM, LTβR and DR3, with keratinocyte-specific conditional knockout mice in models of AD and psoriasis.

Experimental Design: 1.1. LIGHT and TL1A co-operate to promote hyperplasia and inflammatory features in human keratinocytes: Firstly, the inventors test how signaling from LIGHT and TL1A directly regulate keratinocytes in vitro. Studies are performed with titrated doses of rLIGHT or rTL1A (0.1-100 ng/ml) vs. PBS, added to normal human epidermal keratinocytes from a minimum of 4 donors (obtained from Lonza) with kinetic analyses, as described previously (7, 8). LIGHT and TL1A have maximal activity in the range 20-100 ng/ml, similar to IL-13 or IL-17. The inventors have shown that rLIGHT can induce proliferation/hyperplasia of human keratinocytes via both HVEM and LTβR. The inventors found TL1A could induce NF-κB activation in keratinocytes (FIG. 2C), a pathway that often is involved in cell division. The inventors determine rTL1A alone displays a similar activity to LIGHT, and test this with BrdU, thymidine incorporation, and cell cycle flow analyses. The inventors control for specificity with siRNA knockdown of DR3, and then determine synergistic or additive activities, cross-titrating rLIGHT together with rTL1A and measure the extent of cell division and growth. The inventors also test if co-operation is at the level of the receptors (e.g. TL1A enhances expression of the LIGHT receptors).

The inventors then test if rLIGHT or rTL1A alone induce inflammatory activity of relevance to either AD and/or psoriasis, and the extent to which they co-operate and synergize. The experiments are conducted in an unbiased manner with RNA-seq. This provides direct comparison between LIGHT and TL1A and shows common as well as possibly distinct transcriptional targets in keratinocytes. LIGHT and TL1A promote known gene targets already described as elevated in AD or psoriatic skin lesions. For this the generated data is compared to RNA-seq and microarray data compiled from skin biopsies taken from AD and psoriasis patients (22). This dataset was from 147 samples of AD and psoriasis patients and healthy controls, using newly obtained samples as well as results from 15 prior publications. Showing strong commonality between the two diseases, the authors found 2,800 differentially expressed genes (DEGs) with >1 log 2-fold change (up and down) shared between AD and psoriasis lesional skin that corresponded to 41% and 81% of the psoriasis and AD DEGs, respectively. The inventors have generated a list of 1197 psoriasis DEGs and 1203 AD DEGs with >1 log 2-fold upregulation in lesional skin compared to healthy skin and non-lesional patient skin from this publication, and also including a number of genes from two prior psoriasis studies (61, 70). In psoriasis, these include inflammatory/anti-microbial proteins (S100A7, S100A8, S100A9, S100A12, DEFB4A); serine protease inhibitors (SERPINA1, SERPINB4, SERPINB3, SERPINB13); cytokines (IL36G, IL36A, IL22, IL19, IL17A, TNF, IL23A/p19, IL23p40, IL6, IL1b); chemokines (CXCL13, CXCL9, CXCL10, CXCL1, CXCL2, CXCL3, CXCL5, CXCL8, CXCL16, CCL20); matrix metalloproteinases (MMP9, MMP1, MMP19, MMP28, MMP10, MMP12), and possible autoantigens (LL37, ADAMTSLS, PLA2G4D). In AD, they also include genes in common with psoriasis such as S100A7, S100A8, SERPINB3, IL17A, IL23A, TSLP, IL32, CCL2, CCL5, CXCL2, MMP9, and collagen isoforms COL4A1, 6A1, 21A1, but also AD-specific genes such as IL4, IL13, IL33, CSF1, VEGFA, and TGFB2. This dataset may reflect not only gene transcripts upregulated in keratinocytes but also in other cell types present in the skin biopsies, including dermal fibroblasts. The inventors find a select number of these genes are upregulated by LIGHT and TL1A in keratinocytes.

The inventors stimulate keratinocytes and compare the transcriptional profiles induced by rLIGHT and rTL1A. As well as upregulated genes, the inventors analyze the published data for downregulated genes in psoriatic vs. AD skin compared to healthy or non-lesional skin (22) and assess LIGHT and TL1A-stimulated keratinocytes for these gene sets. Once the transcriptomes of each individual cytokine is profiled, the inventors focus on co-operativity. The inventors initially cross-titrate rLIGHT together with rTL1A and measure select genes by PCR, and then perform more comprehensive analyses with RNA-seq with fixed concentrations that give optimal effects together.

The inventors validate transcriptional targets that are synergistically upregulated or downregulated with further qPCR and protein analyses, particularly on cytokines and chemokines linked to AD or psoriasis. In targeted studies, the inventors have found that rLIGHT can induce molecules from keratinocytes that are upregulated in AD and psoriasis biopsies (TSLP and periostin). In addition, rLIGHT injected s.c. in vivo induced the cytokines IL-33 and IL-25 that are largely epithelial-derived and are upregulated in AD skin lesions (56, 71-75).

The inventors have shown that rLIGHT inducing several chemokines when injected s.c. into mice, such as CCL5 that is associated with AD and psoriasis. The inventors test if both LIGHT and TL1A promote a specific group of chemokines in keratinocytes, and determine if their activities are overlapping, including chemokines relevant for monocytes/macrophages, T cells, eosinophils, and neutrophils, such as CCL2, 3, 5, 7, 11, 13, and CXCL2, 3, 5, 8, 12, 15, as well as CCL17 and CCL20 which are involved in Th2 or Th17 responses (76, 77). The inventors further evaluate whether both molecules can drive production of IL-36- or IL-10-family cytokines (IL-36α/IL-36γ; IL-19/IL-20/IL-22/IL-24) or the expression of receptors for these molecules, all of high relevance for either AD and/or psoriasis. IL-22 is primarily produced by T cells (78, 79), is upregulated in patients with AD (15, 16, 80), and when overexpressed in the mouse skin can induce AD-like symptoms (81). It can also drive proliferation of keratinocytes and upregulate other cytokines or chemokines like IL-20 and CCL17 (82-84). IL-19 and IL-24 are primarily associated with psoriasis, but were upregulated in keratinocytes by IL-4, or in the skin of IL-4 transgenic mice (86), suggesting they could be pathogenic in AD as well. IL-19 and IL-24 may contribute to epidermal hyperplasia (87, 88) and then could, in an autocrine manner, perpetuate the action of LIGHT and TL1A if induced by these molecules. To assess indirect/feedback effects of LIGHT and TL1A, the inventors also inhibit soluble factors in the cultures, such as blocking TSLP, IL-19, or IL-24. Lastly, the inventors study barrier function proteins whose lowered expression is associated with AD or psoriasis such as filaggrin, loricrin, and involucrin. The inventors test whether either cytokine alone or together downregulates these molecules. The inventors similarly assay for tight junction proteins that are deregulated in AD or psoriasis (claudin-1 and -4, occludin, E-cadherin) using IF in air liquid interface 3D cultures (89, 90). Overall, the results in these experiments demonstrate how LIGHT and TL1A transcriptionally regulate keratinocytes and how this correlates with transcriptomes associated with human AD or psoriasis. They furthermore show the extent of overlap between these TNF family proteins and overlap or divergence from the three keratinocyte-acting inflammatory cytokines (TNF, IL-17, IL-13) currently most recognized as being of relevance to AD and psoriasis pathogenesis.

The expression of HVEM, LTβR, and DR3 in keratinocytes is essential for driving skin inflammation in vivo: Here, the inventors show the importance of LIGHT and TL1A to keratinocyte activity in vivo with targeted studies in the mouse. A HDM model of AD is used and an imiquimod model of psoriasis. Skin inflammation is monitored by clinical scoring, histology staining with trichrome or antibodies to collagen I or IV, αSMA, ki67, keratin 1, 5, 14, and 17, and quantitation of dermal and epidermal thickening. The inventors monitor skin infiltrates in sections (toluidine blue, mast cells; congo red, eosinophils; vimentin and CD90 with/without αSMA, myofibroblasts/fibroblasts; Ly6G and myeloperoxidase, neutrophils), and in biopsy by flow cytometry (7, 91). This determines if there are selective effects on accumulation or persistence of Th2/Tc2, or Th17/Th22 αβ T cells, γδ T cells, neutrophils, eosinophils, Ly6C hi/lo monocytes, M1/M2 macrophages, DC subsets, ILC, and mast cells. qPCR analyses are performed on select inflammatory cytokines and chemokines described below. Expression of LIGHT and TL1A is monitored by both bulk mRNA analyses of skin tissue, IF of tissue sections, and flow of individual cell populations. This will determine if there is co-expression of the ligands, differential expression, and kinetic differences in upregulation. The inventors also correlate the expression of LIGHT and TL1A with expression of signature cytokines of AD or psoriasis, namely TSLP, IL-5, IL-9, IL-13, IL-17, IL-22, and IL-23.

The inventors have found that deletion of HVEM in keratinocytes protected mice from developing experimental AD (8). Because one specific knockout in keratinocytes gave a striking phenotype and abolished disease, this does not mean that other molecules do not contribute to keratinocyte activity. The inventors determine that there is cooperation between cytokine receptors to drive inflammatory processes, and moreover receptors also act in a temporal manner one after another. Either could give the result that deletion of each individual receptor results in a phenocopy and strongly reduce disease.

Next, the inventors determine if HVEM expression in keratinocytes is also required for experimental psoriasis with the current K14-cre foxed mice used in the AD studies, and whether LTβR or DR3 are also active and essential in keratinocytes in the AD and psoriasis models by creating conditional knockouts of each of these receptors. With keratinocyte-specific deletion of LTβR or DR3, epidermal hyperplasia is reduced. The inventors also find reduced immune infiltrates as chemokine and cytokine products of keratinocytes that impact migration and maintenance of T cells, etc., are likely to be impaired. As described above, the keratinocyte deletion of HVEM strongly reduced expression of IL-4, IL-5, and IL-13 in the AD model, as did a whole-animal deficiency in LIGHT (8). For LIGHT, these contribute to late immune cell activity through stimulating keratinocytes to produce chemokines that affect the maintenance of effector T cells in the skin.

The inventors extend the above results and test with skin biopsy mRNA studies, as well as IF tissue staining, where there is a defect in vivo in select molecules when HVEM, LTβR, or DR3 activity is absent in keratinocytes. The inventors also compare expression of selected genes from above (e.g. TSLP, IL-33, periostin, IL-36γ, CCL5, CCL17, CCL20, CCL26, IL-19, IL-24, loricrin, involucrin, and filaggrin) in skin biopsies from keratinocyte-specific knockouts to those in LIGHT vs. DR3 whole-animal knockouts in the two models. This allows the inventors to understand if cells other than keratinocytes contribute to the LIGHT- or TL1A-dependent activity in the skin. The inventors further validate key downstream molecules, and co-operativity or synergy, by injecting s.c. titrated amounts of the recombinant cytokines (0.1 to 10 μg), separately and together, vs. PBS, into WT and K14-creReceptor-floxed mice and assessing induction of mRNA and protein. Periostin has been validated as an in vivo target of LIGHT-HVEM interactions in keratinocytes. The inventors additionally inject rLIGHT into DR3−/− mice vs. K14-creDR3-flox mice, and rTL1A into LIGHT−/− vs. LTβR- and HVEM-flox mice to directly test if one is downstream, or simultaneously required, for skin inflammatory activity driven by the other molecule.

What are the primary signaling pathways induced by LIGHT and TL1A in keratinocytes that promote inflammatory factors? The inventors build on the understanding of the similarities between LIGHT and TL1A by assessing their signaling pathways in human keratinocytes. These receptors have been described in various cell types to activate canonical and non-canonical NF-κB, JNK/AP1, ERK and p38 MAPK pathways. Early studies (96, 97) suggested LTβR primarily activates NIK/IKKα-dependent non-canonical NF-κB, and HVEM IKKβ-dependent canonical NF-κB, and DR3 has largely been linked to canonical NF-κB (98), but this is likely too simplistic (99-101). Furthermore, new activities via PI3K/Akt have been reported for HVEM, LTβR, and DR3 (102-105). The inventors determine that signaling by one or several of these pathways (NF-κB, JNK, ERK, Akt, and p38) regulates production of cytokines and chemokines in keratinocytes (106, 107). One or two molecules can be focused downstream of both LIGHT and TL1A (e.g. TSLP, periostin, CCL5) as well as any molecules that might be induced by each protein. The inventors perform inhibitory studies with inhibitors of canonical NF-κB (to block phosphorylation of IKKα or IKKβ: Bay 11-7082, Bay 11-7085, PF184) and non-canonical NF-κB (to block NIK: NIK-SMI1), and JNK (SP600125), p38 MAPK (SB203580), ERK1/2 (FR180204, U0126), and PI3K/Akt (LY294002, Akti1/2). The inventors correlate inhibitor-suppression of cytokine/chemokine production with a direct activity of LIGHT or TL1A in promoting these signaling pathways (by assessing the phosphorylated forms, or with kinase assays e.g. pAkt, pIKK, pJNK, conversion of p100 to p52 for non-canonical NF-κB, nuclear accumulation of p65 and p50 for canonical NF-κB). The inventors confirm data with more applied studies, e.g. with retroviral or lentiviral transfection of a phosphorylation-deficient mutant of IκBα (IκBαSR); or siRNA for NIK (NF-κB-inducing kinase). Further studies focus on dissecting how LIGHT synergizes with TL1A. The inventors determine whether any common targets and synergy of LIGHT and TL1A are controlled by quantitative signaling through core pathways which may be canonical NF-κB for molecules such as TSLP, and non-canonical NF-κB for chemokines (both supported by basic signaling studies). The inventors also determine whether differential induction of any molecule discovered is under the control of specific MAP kinase pathways unique to LTβR, HVEM, or DR3.

To Identify the Inflammatory Activities of LIGHT and TL1A in Dermal Fibroblasts:

Rationale: Fibroblasts can proliferate extensively and undergo myofibroblast differentiation, leading to skin structural rigidity, involving upregulating proteins such as alpha smooth muscle actin (αSMA). They can also produce ECM proteins like collagen, and other proteins like periostin, that contribute to dermal tissue dysregulation. Fibroblasts additionally can make chemokines, and be a source of inflammatory cytokines such as IL-6, TNF, IL-33, IL-24, IL-19 (and in some cases IL-23 (129, 130)) that can further modulate immune cell activity and/or act on keratinocytes to maintain or enhance the epidermal response (68, 69, 131-136). Given receptor expression on fibroblasts that have discovered, that the inventors find that LIGHT and TL1A drive accumulation of fibroblasts and promote inflammatory activity that fuels continued immune cell infiltration in the skin and helps to maintain keratinocyte dysregulation, reinforcing the direct signals keratinocytes receive from these cytokines. The inventors test this in vitro with human dermal fibroblasts, and in vivo with fibroblast conditional knockouts of HVEM, LTβR, and DR3. The inventors further determine whether the same signaling cascades are active in fibroblasts as in keratinocytes, and if LIGHT and TL1A control similar or distinct functions in these two divergent cell types.

LIGHT and TL1A synergistic in driving proliferation and inflammatory activity in dermal fibroblasts: The inventors determine inflammatory events that are triggered by LIGHT and TL1A in normal human dermal fibroblasts, using cells from a minimum of 4 donors, with kinetic analyses. The inventors find that both LIGHT and TL1A induce fibroblast proliferation, and may have differential abilities to induce myofibroblast differentiation. In preliminary studies, the inventors found that rLIGHT upregulated TSLP, periostin, and αSMA, in dermal fibroblasts, indicative of the latter, and this is confirmed with additional replicate experiments. The inventors also test a range of inflammatory factors relevant to AD or psoriasis that have receptors on fibroblasts (e.g. LIGHT, IL-13, TGF-β, FGF, TNF, IL-17) to determine if they can promote DR3. The inventors have found activities of TL1A in lung fibroblasts (3) that suggest that similar effects can be discovered in dermal fibroblasts if DR3 is adequately expressed. rTL1A induced proliferation but not αSMA in lung fibroblasts, and it synergized with TGF-α, a primary factor that can promote myofibroblast differentiation, for both activities. Moreover, rTL1A alone induced periostin and collagen (3).

The inventors first titrate (0.1-100 ng/ml) the recombinant proteins vs. PBS on dermal fibroblasts and determine if LIGHT and TL1A promote proliferation (BrdU, thymidine incorporation, and cell cycle analysis) and/or myofibroblast differentiation (upregulation of collagen isoforms, tenascin, periostin, and αSMA). Next, the inventors perform RNA-seq and determine the extent of overlap and divergence in genes targeted by LIGHT vs. TL1A. Using the lists of DEGs upregulated and downregulated in psoriasis or AD biopsies, the inventors determine how many of these transcripts are induced or suppressed in dermal fibroblasts by LIGHT or TL1A, and compare this to the actions of TNF, IL-13, and IL-17 that also have receptors on fibroblasts. In addition, the inventors analyze the combined RNA-seq data for DEGs in fibroblasts vs. keratinocytes that are promoted by either LIGHT or TL1A. The inventors find that LIGHT and TL1A overlap to a significant extent in their targets in fibroblasts and there is a common core transcriptional signature induced by both molecules in fibroblasts and keratinocytes as well as a unique signature in each cell type. The inventors further test if LIGHT and TL1A can induce adhesion molecules and chemokines relevant for skin localization of fibroblasts themselves, or aid migration of eosinophils, neutrophils, and T cells. In published studies (41, 42), the inventors found that rLIGHT upregulated some chemokines in lung fibroblasts similar to lung epithelial cells (CCL5, CCL20 CXCL5 CXCL11), supporting the hypothesis that LIGHT and TL1A may induce an overlapping profile of chemokines in dermal fibroblasts compared to keratinocytes. The inventors assess control of fibroblast migration where a scratch is made in monolayers of stimulated fibroblasts and their ability to migrate into the scratch is measured (137). These conclusions are enhanced by assessing fibroblast motility using 3D matrigel models (BD Biosciences). The inventors determine which out of the chemokines identified is responsible for motility by application of specific blocking antibodies. To assess direct vs. indirect effects, the inventors also block certain molecules in vitro, such as TGF-α that have been described to promote expression of periostin and a SMA in fibroblasts. Where α SMA is induced, in additional mechanistic studies, the inventors perform collagen gel contraction assays to determine the impact on fibroblast contractility (138, 139). Lastly, the inventors assess synergistic or additive activities of LIGHT and TL1A when acting together, showing that strong synergy is exhibited for genes dependent on canonical and non-canonical NF-κB activity.

Does fibroblast expression of HVEM, LTBR, or DR3 contribute to AD or psoriasis in vivo? The inventors further test whether fibroblasts are direct cellular targets of LIGHT and TL1A in vivo using mice where HVEM, LTβR, or DR3 are conditionally deleted in these cells. The inventors pursue deletion of each receptor if the in vitro studies show a strong effect of all three receptors in dermal fibroblasts. The inventors firstly target fibroblasts by crossing to Col1α2-cre mice (already in-house) given the broad expression of Col1α2 in most fibroblasts, and assess the overall skin inflammatory response in the models of AD and psoriasis. Alternatives are to use FSP1 (S100α4)-cre mice as done by others (140, 141), although FSP1 can be expressed in cells other than fibroblasts, or use α-SMA-cre mice that allow targeting of myofibroblasts. Fibroblasts can be distinguished as CD45⁻Epcam⁻CD31⁻Vimentin⁺CD90⁺ cells (3). This analysis was previously used to confirm constitutive expression of DR3 (and HVEM and LTβR) on fibroblasts, and the idea that DR3 was active under conditions of inflammation because TL1A was expressed by macrophages only with challenge with an epithelial insult (bleomycin) or an allergen (HDM). The inventors perform similar phenotypic studies in mouse skin for DR3, HVEM, and LTβR on dermal fibroblasts and also assess expression of TL1A and LIGHT on various inflammatory infiltrates after HDM or imiquimod challenge, including macrophages which are thought important to both AD and psoriasis (142-144). The inventors then monitor steady state and inflammation-induced accumulation of fibroblasts in WT vs conditional KO mice by flow cytometry in skin biopsies as well as by IF microscopy in tissue sections, using αSMA staining to further distinguish and enumerate myofibroblasts from inflammatory fibroblasts.

Dermal thickening is a primary feature of the AD model, and the studies described herein show that fibroblast expression of HVEM, LTβR, and DR3 is a major contributor to this process. Dermal thickening is not seen in the psoriasis model, but strong infiltration of immune cells is common to both and a lack of chemokine production by fibroblasts will alter the balance of the infiltrates. Tissue infiltration of CD4 and CD8 T cells, γδ T cells, ILCs, monocytes/macrophages, mast cells, etc., show there is a selective effect on recruitment or maintenance of certain cell types that could be linked to chemokine expression. A lack of HVEM, LTβR, or DR3 expression in fibroblasts does directly affect initial generation or differentiation of CD4 or CD8 T cells, but this may affect T cell accumulation or maintenance over time due to defective chemokine production, and is tested in studies described above. The inventors also show that epidermal hyperplasia in these models is diminished because of lower levels of fibroblast-derived cytokines such as IL-19, TNF, IL-23 and others, that can contribute directly or indirectly to deregulated keratinocyte activity. Primary inflammatory factors that are reduced when each receptor is not expressed in fibroblasts are also investigated. This is done in parallel with studies in 2.1, and again focus on molecules associated with fibroblast activity (e.g. CCL2, 5, 17, 20; TSLP, IL-19, IL-24). The inventors compare skin biopsy mRNA profiles between the fibroblast-specific knockouts and the keratinocyte-specific knockouts, to further understand the relative contributions of keratinocytes and fibroblasts to individual inflammatory factors in the disease setting. These studies demonstrate the importance of fibroblast expression of LIGHT and TL1A's receptors to skin inflammation and provide direct insight into the role of fibroblasts in skin disease.

Therapeutic Targeting of LIGHT and TL1A can Block or Reverse Progression of Skin Inflammatory Disease

Rationale: The inventors determine that therapeutic targeting of LIGHT/HVEM, LIGHT/LTβR, and TL1A/DR3 interactions, alone or together, suppress ongoing skin inflammatory disease. Both cytokines act within the skin, directly driving skin pathology through activities on keratinocytes and fibroblasts, and they maintain skin inflammation through promoting chemokines or inflammatory cytokines by keratinocytes and fibroblasts that either feed forward to drive continued keratinocyte activity and/or allow the continued persistence and activities of infiltrated immune cells in the dermis or epidermis. The studies here support these ideas. The inventors block LIGHT and TL1A in therapeutic protocols with extended and longer-term AD and psoriasis. Fully human neutralizing antibodies to LIGHT (KHK/SAR252067; Kyowa Kirin Co (158)) and TL1A (PF-06480605; Pfizer (159)) have already been generated and are in trial or being considered for therapy of ulcerative colitis. Importantly, embodiments of the present disclosure describe bispecific blocking reagents capable of neutralizing both human molecules.

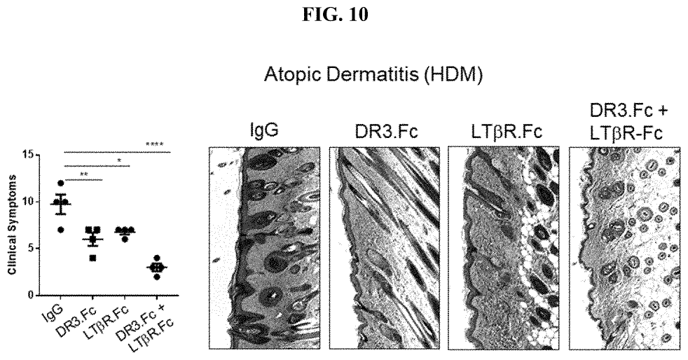

Experimental Design: The inventors use several reagents: a pan LIGHT blocker, LTβR.Fc, that was used in severe asthma studies (1) that inhibits LIGHT binding to both HVEM and LTβR; specific antibodies to LTβR and HVEM that only block LIGHT binding to each respective receptor (2, 8, 160, 161); and a TL1A blocking DR3.Fc fusion protein previously used in the inventors' lung inflammation studies (3). The antibodies/fusion proteins or control Ab/Fc are administered therapeutically (200 μg every 3 days) in the AD and psoriasis models after skin disease has been established. In the AD model, treatment first starts at day 7 when disease is already strongly evident after one cycle of HDM treatment, and the inventors then assess skin inflammation after the second cycle of HDM exposure on day 14, 20, and 30 to monitor progression or maintenance of disease. The inventors generated supporting data reinforcing these studies and providing that blocking both molecules together is likely to be more therapeutically beneficial than blocking each molecule separately (FIG. 10). The inventors extend the in vivo AD protocol to assess therapeutic activity where disease has already been established for some time and use repeated treatments of the skin with allergen to in part mimic chronic cyclic disease seen in human AD. For example, the inventors perform three or four cycles of allergen exposure over 21 or 28 days, with therapeutic antibody treatment starting after the second cycle or third cycle, respectively. The inventors are more restricted in the imiquimod model as this is a self-limiting model. The inventors have established that maximal disease is seen at day 6 with imiquimod treatment every day, and that this skin reaction is maintained at the same level if treatment is extended until day 12. Termination of treatment at 12 days, or continued treatment beyond this time, results in slow resolution of disease over the next 8-12 days. The inventors then perform therapeutic blocking starting on day 7 through to day 11, and assess disease on day 12 and 20. The inventors also start blocking on day 10 and monitor for quicker resolution of disease over that which occurs naturally in the next 10 days.

In all cases, the inventors block LIGHT-LTβR vs. LIGHT-HVEM, as well as perform combination treatment with each antibody together with DR3.Fc, to determine if there is a qualitatively different effect neutralizing one or the other of LIGHT's receptors. As described above, in separate experiments in WT mice, the inventors monitor expression of LIGHT and TL1A in the skin by mRNA and flow analyses over time and do this in the normal as well as extended models. Combined studies demonstrate when LIGHT and TL1A are active, which receptors are crucial, and that LIGHT and TL1A function to maintain skin disease over time. Additionally, the studies demonstrate neutralization results in faster kinetics of resolution of disease where this occurs spontaneously. The inventors repeat allergen or imiquimod treatments at later times to determine prior blocking provides a tolerogenic effect and limits future episodes of disease, including functional assessments of the T cell response and other infiltrates into the skin.

### Example 2

Peribronchial fibrosis and smooth muscle remodeling are primary characteristics of severe asthma and are associated with decline in airway function and reduced bronchodilator response. In the studies, the inventors have demonstrated the overall importance of two TNF family cytokines, LIGHT (TNFSF14) and TL1A (TNFSF15), to airway remodeling in mouse models of severe asthma driven by house dust mite allergen. Moreover, the inventors have found that both cytokines are key to development of skin tissue remodeling reminiscent of atopic dermatitis, in a mouse model also driven by allergen. LIGHT and TL1A are made by several immune cells, either T cells, macrophages, dendritic cells, eosinophils, or neutrophils. Their receptors (LTβR and HVEM for LIGHT; DR3 for TL1A) are found on varying hematopoietic cells, but the inventors found that injection of either recombinant TL1A or LIGHT into the airways of naïve mice rapidly promotes smooth muscle alterations, collagen deposition, and airway hyperresponsiveness, suggesting important direct effects on airway structural cells. In line with this, the inventors discovered that the receptors for LIGHT and TL1A are expressed on human airway fibroblasts and smooth muscle cells, two of the cell types that contribute to the in vivo pulmonary phenotypes that are regulated by LIGHT and TL1A. Moreover, the data show that LIGHT and TL1A can induce pro-fibrotic and inflammatory activities from these cells that are highly relevant to bronchoconstriction and airway remodeling.

As airway fibroblasts and smooth muscle cells are thought primary contributors to airway rigidity, lack of plasticity, and abnormal airway responsiveness, and these two critical cell types are dysregulated in severe asthma. Without being limited to a particular theory, LIGHT and TL1A form a network of soluble cytokines to drive pathogenic phenotypes in these cells. Defining how LIGHT and TL1A integrate with other mediators to control fibroblast and smooth muscle cell remodeling activities provides new combination therapeutic approaches to limit severe asthma, as disclosed herein.

In some instances, recombinant TL1A and LIGHT can promote increases in airway collagen and smooth muscle in vivo, and that blocking both molecules can reduce allergen-induced airway remodeling. For example, neutralizing reagents to LIGHT and TL1A are utilized to understand the potential for combined therapeutic treatments in limiting allergen-induced airway remodeling. Failed trials blocking IL-4 or IL-13 alone, but success with dupilumab that blocks both molecules, has shown that combination targeting approaches in asthma are acceptable and likely to create greater benefit than monotherapy. Thus, an understanding of the biology of LIGHT and TL1A and how they synergize with other cytokines to promote remodeling features may lead to novel combination treatments for severe asthma. Fully human neutralizing antibodies to LIGHT (KHK/SAR252067; Kyowa Kirin Co (93)) and TL1A (PF-06480605; Pfizer (94)) have already been generated and are in trial or being considered for therapy of ulcerative colitis.

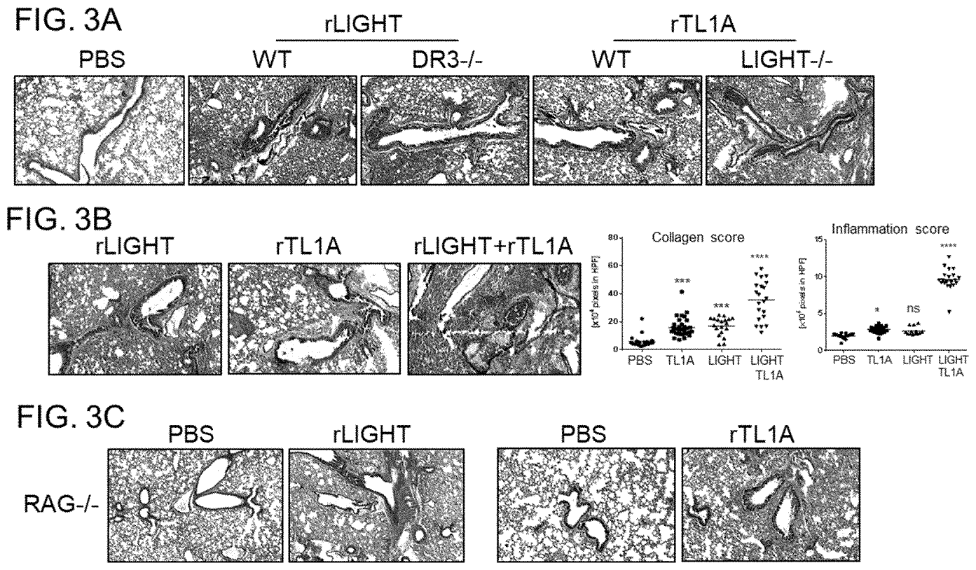

LIGHT acts through two receptors in the TNFR superfamily, namely HVEM (TNFRSF14) and LTβR (TNFRSF3). LIGHT is not constitutively produced but can be transiently induced in activated T cells primarily as a soluble cytokine. It has also been found in some instances to be inducible in NK cells, DCs, macrophages, eosinophils and neutrophils. TL1A is similarly an inducible molecule, also likely acts primarily as a soluble cytokine, and can be made by DCs, macrophages, fibroblasts, epithelial cells, neutrophils and eosinophils (44, 46, 47). TL1A acts through the receptor DR3 (TNFRSF25). With regard to LIGHT, the inventors have linked it to asthma by showing that LIGHT signaling via HVEM that is expressed on Th2 memory cells promoted their survival and accumulation in the airways, and thus controls airway inflammation via regulating the availability of IL-5 and IL-13 (49). The inventors then demonstrated that endogenous LIGHT production in the mouse contributes to airway remodeling in a manner distinct from regulating Th2 cells. In a house dust mite-driven model of severe asthma that is characterized by upregulation of type 2 cytokines (IL-5, IL-13), as well as IL-17 (48), and in a bleomycin-driven model of pulmonary fibrosis (54), LIGHT-deficient mice were protected from accumulation of peribronchial and perivascular collagen and bronchiole-locating cells expressing αSMA (mature smooth muscle and myofibroblasts). This LIGHT-deficient phenotype was further reproduced with semi-therapeutic treatment with a neutralizing LTβR.Ig fusion protein that blocks LIGHT interacting with LTβR and HVEM (e.g. FIG. 3A (48)). The inventors further showed that LIGHT was also active, co-operating with TGF-β, to drive airway remodeling during rhinovirus infection in the mouse (58)

For example, LIGHT−/− mice displayed reduced thickening of the skin, less collagen in the adipose layers, and fewer αSMA+keratinocytes and myofibroblasts in models of atopic dermatitis and scleroderma (FIG. 2b, (55, 59)). The inventors have also expanded the concept of LIGHT being central to tissue remodeling, finding it crucial for skin thickening in the imiquimod-driven model of psoriasis. This suggests that LIGHT activity is not confined to only Th2 responses but it can be relevant for Th17/Th1 responses, of potential significance for severe asthmatics that might display the latter phenotypes (70-72).

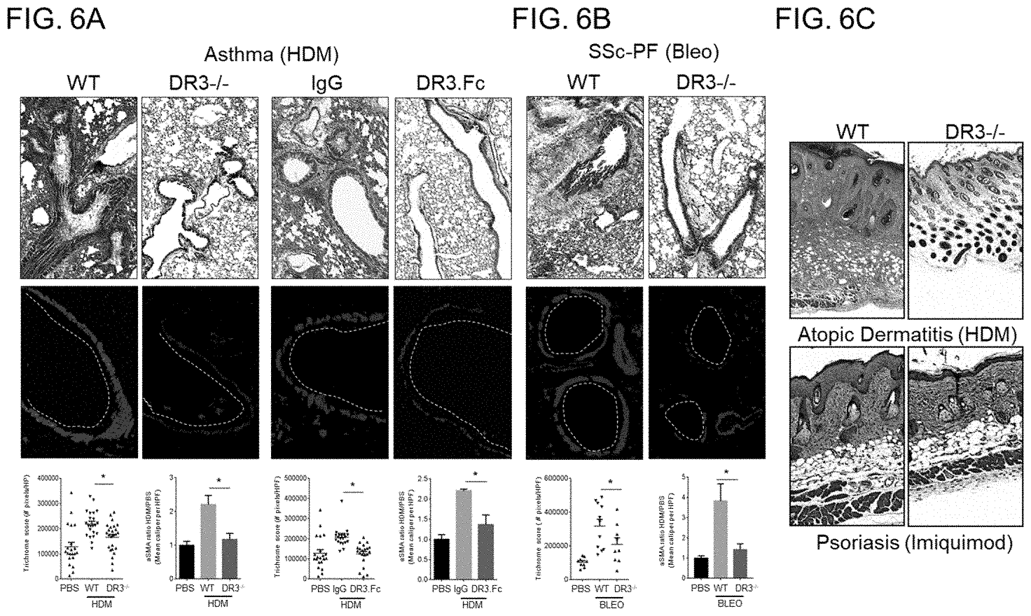

While performing these studies, the inventors have discovered that TL1A was also upregulated in the airways of HDM or bleomycin challenged animals, and with DR3-deficient mice, it was found that it was also essential for tissue remodeling in the airways and skin in the same animal models of severe asthma, scleroderma, atopic dermatitis, and psoriasis where LIGHT is active (FIG. 6A-6C, (69).

Blocking TL1A after acute sensitization in the severe asthma model, with an Ig fusion protein of DR3, also inhibited airway remodeling similar to blocking LIGHT (69). The inventors furthered these observations by testing whether LIGHT and TL1A could induce fibrosis and remodeling independently of other inflammatory activity, and of each other, by injecting the recombinant proteins into naïve mice. Importantly, both molecules induced substantial collagen deposition and αSMA accumulation in the airways as well as the skin (FIG. 6A-6C, (54, 55, 69)). Moreover, the effects of LIGHT and TL1A were not reliant on the other cytokine shown by injection of the proteins into the respective knockout animals. Each molecule also induced remodeling in RAG and RAGγc−/− mice, showing activities independent of T cells and ILCs (FIG. 3A-3C). This suggests that these are separate but complementary functions of the two molecules, and demonstrating that the combination of rLIGHT with rTL1A induced greater collagen deposition in the airways and inflammation (FIG. 3B).

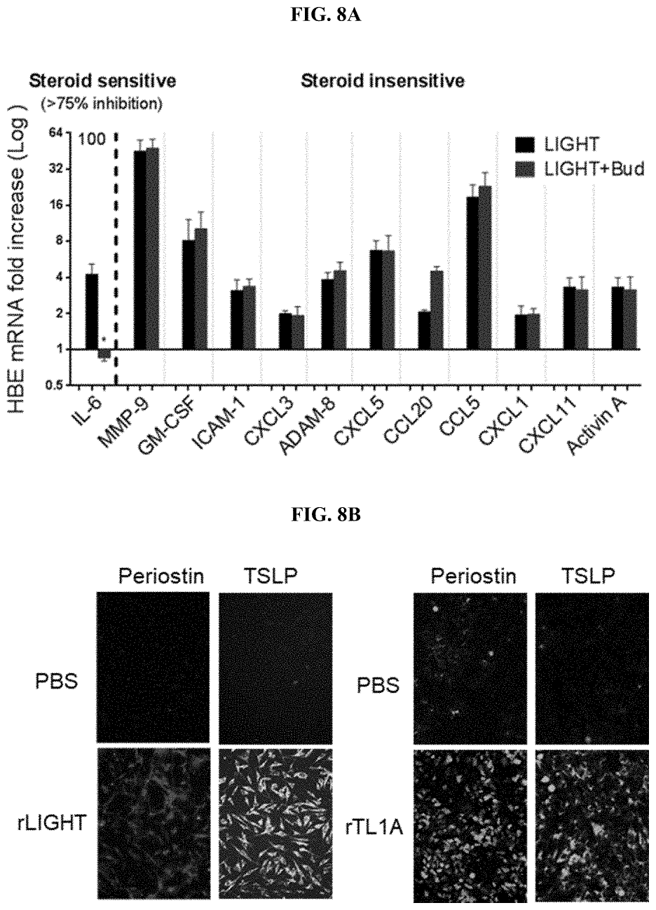

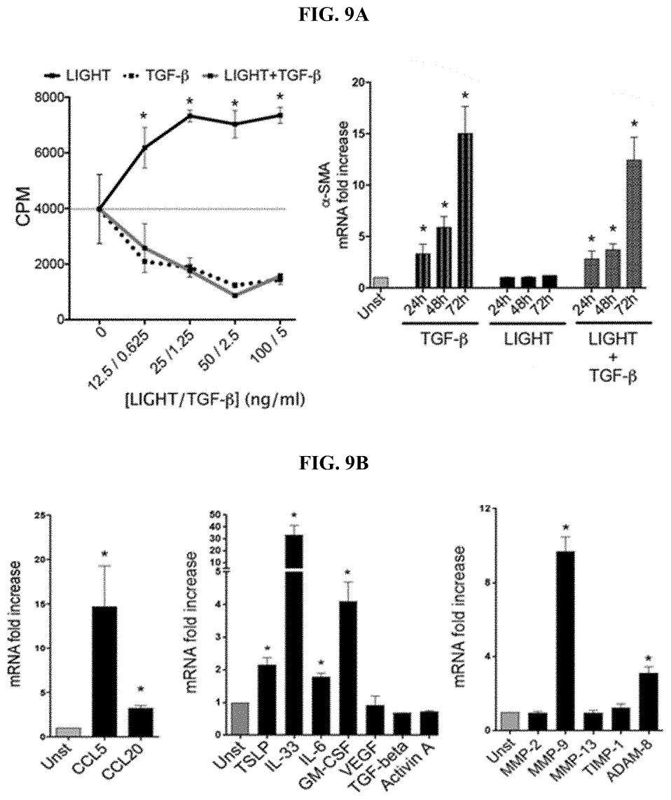

The inventors show that both LIGHT and TL1A are mediators of airway tissue remodeling as they directly regulate functional activity in airway structural cells. The inventors have already gathered much data on human bronchial epithelial cells. rLIGHT can induce expression of adhesion molecules (ICAM-1, VCAM-1), proteinases (MMP-9, ADAM-8), cytokines (activin A, GM-CSF), and chemokines (CCL5, CCL20, CXCL1, CXCL3, CXCL5, CXCL11) in a steroid-resistant manner, of particular significance to severe asthma (FIG. 8A, and (53)). Furthermore, TSLP and periostin were upregulated by LIGHT in HBE (FIG. 8B, (54)) and it was found that TL1A also upregulated these molecules (FIG. 8B, (69)).

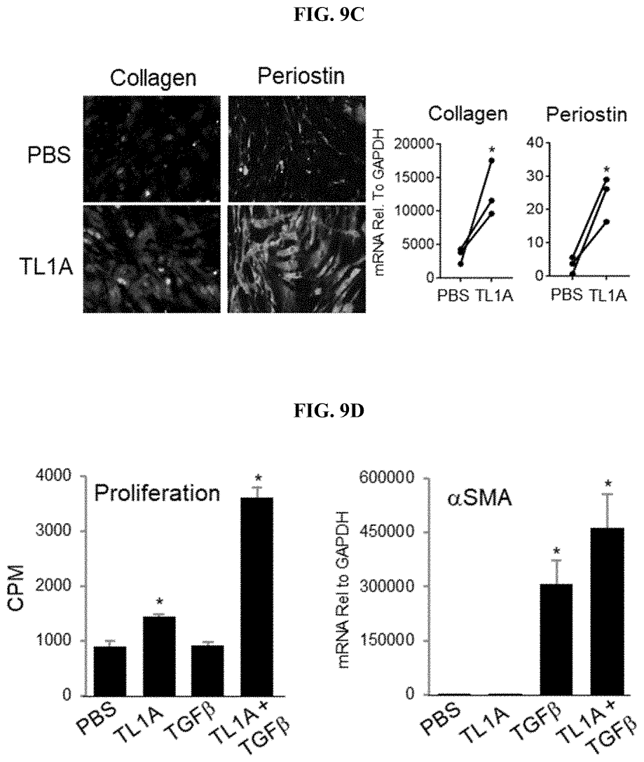

The inventors show that signaling through LTβR, HVEM, and DR3 directly regulates inflammatory events in HAF and that their individual activities drive unique phenotypes marked by differential gene expression. The inventors compare rTL1A to rLIGHT, titrating each molecule (5-100 ng/ml, their optimal dose range, see (60, 69)). The inventors perform kinetic analyses over 3 days (e.g. 4, 24, 48, 72 hr) and compare data from 4-6 individual cell populations. The inventors initially gain data with normal HAF. The inventors have shown that LIGHT can promote proliferation of HAF but cannot drive myofibroblast differentiation (upregulation of αSMA), activities divergent from TGF-β (FIG. 9A). The inventors also found that LIGHT induced adhesion molecules (ICAM1, VCAM1), chemokines (CCL5, CXCL5, 11, 12), cytokines (TSLP, IL-33, IL-6, GM-CSF) and metalloproteases (MMP-9, ADAM8), through the LTβR, shown with siRNA knockdown (FIG. 9B, and (60)). The inventors additionally have shown that rTL1A can promote activities in HAF that might overlap with, but also be distinct from, LIGHT (FIGS. 9C and 9D). This included promoting collagen 13 and periostin, the former shared with LIGHT but not the latter (FIG. 9C). TL1A also induced proliferation of HAF, similar to LIGHT, but unlike with LIGHT, TL1A-driven proliferation was augmented by TGF-β and TL1A enhanced TGF-β driven αSMA (FIG. 9D).

### Example 3

The inventors have validated therapeutics blocking two TNF Superfamily members LIGHT and TL1A in fibrotic diseases relevant to Systemic sclerosis (aka Scleroderma or SSc) and Pulmonary Fibrosis (PF) in humans.

Due to the lack of prognostic biomarkers, SSc-PF displays a poor prognosis in clinical practice. Moreover, there is an unmet need to develop anti-fibrotic drugs, since anti-inflammatory remedies have so far failed to reduce fibrosis and tissue remodeling in the clinic. Alternatively, five TNF inhibitors have been FDA approved to treat different inflammatory diseases presented with fibrosis and are safe to administer to patients. The inventors have shown that blocking either LIGHT or TL1A signaling in isolation has proven efficient in reducing fibrosis associated with SSc-PF. LIGHT plays a central role in lung and skin fibrosis since it can control the expression of major pro-fibrotic factors such as TSLP, IL-13, TGF beta, and the extracellular matrix protein Periostin. When LIGHT signaling is disrupted by genetic deletion of LIGHT (or one of its receptors) or antibody blocking, significant reduction in alpha-smooth muscle actin (alpha-SMA) and collagen depositions are observed. Moreover, LIGHT alone can induce a fibro-proliferative disorder that mimics human SSc-PF in the lung and skin; indeed, when administered alone into the airways or subcutaneously, LIGHT increases the accumulation of collagen and alpha-SMA leading to fibrosis.

The inventors have discovered another TNF family Member called TL1A can also potentiate fibrosis similarly to LIGHT and which signaling can promote tissue remodeling. In the present disclosure, the inventors show that LIGHT and TL1A synergize together without being inter-dependent in promoting fibrosis. By acting directly on structural cells like fibroblasts, LIGHT and TL1A promote remodeling aside from their pro-inflammatory role. When given in concert, LIGHT and TL1A maximize inflammation and fibrosis. Therefore, blocking concomitantly both LIGHT and TL1A activities post-disease onset. Using antagonistic fusion proteins to neutralize LIGHT and TL1A signaling, the inventors treated mice exposed to allergens epicutaneously to drive atopic dermatitis, post-disease onset. The combination therapy neutralizing LIGHT and TL1A was able to regress fibrosis of the skin, and decrease the clinical symptoms of eczema, namely eruption, scaling, bleeding and redness. Epidermal thickening was drastically reduced when the signaling of both molecules was interrupted, as well as collagen deposition in the dermis.

The inventors extended these findings to treat two models of skin and lung fibrosis induced by bleomycin after the disease is established. The inventors also tested whether maintenance therapy is required to prevent the disease from re-stemming, once the treatment is interrupted. An inflammatory/fibrotic signature of both TNF molecules on primary human fibroblasts was isolated from diseased individuals, and major players of SSc-PF. The biological targets downstream of LIGHT and TL1A serve as theranostic markers to monitor responses to targeted therapies. The expression of LIGHT, TL1A and their receptors in the serum and skin biopsies of SSc-PF patients was analysed via slow versus rapid progressors (matched with healthy control) over the course of a year. This longitudinal evaluation of these molecules on human specimens shows they can serve as a molecular classification to predict disease severity and match appropriately treatments to patients. Thus, the inventors have validated novel therapeutics targeting LIGHT and TL1A in two different murine models of SSc-PF. LIGHT, TL1A and their receptors can serve as prognostic markers to evaluate disease severity on human specimens from SSc-PF patients. Finally, biological signature downstream of LIGHT and TL1A was identified on SSc-PF fibroblasts, leading to potential theranostics that can measure, monitor responses to targeted therapies, define novel regulators of fibrotic activity and enhance the understanding of the pathogenesis of fibrosis. The relevance of this work to Scleroderma and Pulmonary fibrosis therapies are tremendous.

The inventors have initially shown that LIGHT or TL1A blockade in isolation can prevent lung and skin fibrosis in models of systemic sclerosis and idiopathic pulmonary fibrosis induced by bleomycin, as well as asthma and eczema induced by exposure to allergens. LIGHT or TL1A administered solely into the airways or skin, induced a fibroproliferative syndrome, leading to smooth muscle and collagen deposition in the lungs and skins. These findings were extended to demonstrate both molecules were not inter-dependent in driving disease. LIGHT airway administration in a host lacking an active TL1A signal maintains a fibroproliferative phenotype, and vice versa, TL1A administration to LIGHT-deficient mice phenocopied wild type controls. This suggests both molecules act separately to drive fibrosis. Moreover, the inventors have shown that the concomitant administration of LIGHT and TL1A maximized fibrosis in both the lung and skin, inducing passive inflammation and collagen deposition. In a model of eczema induced by epicutaneous exposure to allergen, the inventors demonstrated that the dual blockade of LIGHT and TL1A post-disease onset was able to regress fibrosis of the skin, and the overall clinical symptoms of atopic dermatitis. Collagen deposition in the dermis as well as epidermal thickening were reduced when LIGHT and TL1A signaling were interrupted by administration of the antagonistic reagents LTβR-Fc and DR3-Fc, after the disease is established. These findings were extended to other models of skin fibrosis (bleomycin-induced scleroderma) and lung fibrosis (bleomycin-induced IPF and allergen-induced asthma).

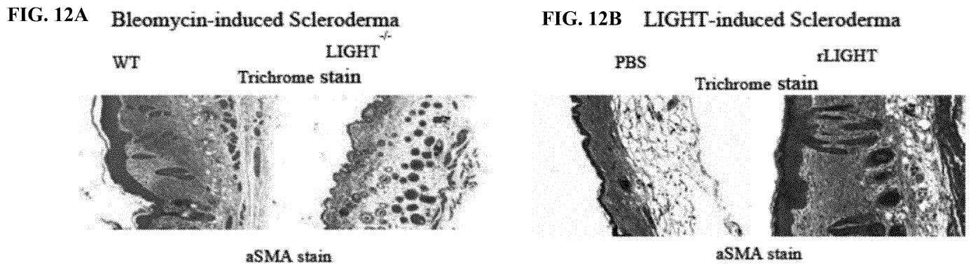

FIG. 12A and FIG. 12B illustrate LIGHT-deficiency decreases in scleroderma skin fibrosis induced by bleomycin whereas LIGHT intradermal injection alone induces scleroderma.

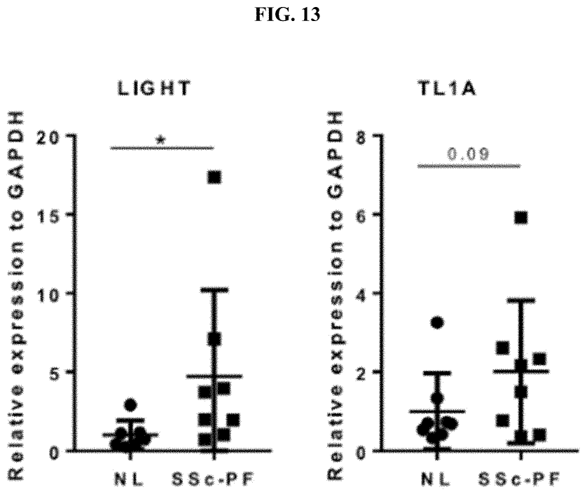

FIG. 13 shows unregulated expression of LIGHT and TL1A transcripts in 8 SSc-PF patients compared to 8 Normal Lungs from healthy individuals.

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES FOR EXAMPLE 1

1. Doherty T A, Soroosh P, Khorram N, Fukuyama S, Rosenthal P, Cho J Y, Norris P S, Choi H, Scheu S, Pfeffer K, Zuraw B L, Ware C F, Broide D H, Croft M. The tumor necrosis factor family member LIGHT is a target for asthmatic airway remodeling. Nature Medicine. 2011; 17(5):596-603. doi: nm.2356 [pii]10.1038/nm.2356. PMCID: 3097134.
2. Herro R, Da Silva Antunes R, Aguilera A R, Tamada K, Croft M. Tumor necrosis factor superfamily 14 (LIGHT) controls thymic stromal lymphopoietin to drive pulmonary fibrosis. J Allergy Clin Immunol. 2015; 136(3):757-68. doi: 10.1016/j.jaci.2014.12.1936. PMCID: 4532661.
3. Herro R, Miki H, Sethi G S, Mills D, Mehta A K, Nguyen X X, Feghali-Bostwick C, Miller M, Broide D H, Soloff R, Croft M. TL1A Promotes Lung Tissue Fibrosis and Airway Remodeling. J Immunol. 2020; 205(9):2414-22. doi: 10.4049/jimmunol.2000665. PMCID: PMC7577982.
4. Croft M. Co-stimulatory members of the TNFR family: keys to effective T-cell immunity? Nature reviews Immunology. 2003; 3(8):609-20. doi: 10.1038/nri1148.
5. Croft M, Benedict C A, Ware C F. Clinical targeting of the TNF and TNFR superfamilies. Nature reviews Drug discovery. 2013; 12(2):147-68. doi: 10.1038/nrd3930. PMCID: 3625401.
6. Croft M, Duan W, Choi H, Eun S Y, Madireddi S, Mehta A. TNF superfamily in inflammatory disease: translating basic insights. Trends in immunology. 2012; 33 (3): 144-52. doi: 10.1016/j.it.2011.10.004. PMCID: 3299395.
7. Herro R, Antunes Rda S, Aguilera A R, Tamada K, Croft M. The Tumor Necrosis Factor Superfamily Molecule LIGHT Promotes Keratinocyte Activity and Skin Fibrosis. J Invest Dermatol. 2015; 135(8):2109-18. doi: 10.1038/jid.2015.110. PMCID: 4504809.
8. Herro R, Shui J W, Zahner S, Sidler D, Kawakami Y, Kawakami T, Tamada K, Kronenberg M, Croft M. LIGHT-HVEM signaling in keratinocytes controls development of dermatitis. J Exp Med. 2018; 215(2):415-22. doi: 10.1084/jem.20170536. PMCID: PMC5789407.
9. Soumelis V. TSLP: from allergy to vaccine adjuvant. Eur J Immunol. 2012; 42(2):293-5. doi: 10.1002/eji.201142337.
10. Allakhverdi Z, Comeau M R, Jessup H K, Yoon B R, Brewer A, Chartier S, Paquette N, Ziegler S F, Sarfati M, Delespesse G. Thymic stromal lymphopoietin is released by human epithelial cells in response to microbes, trauma, or inflammation and potently activates mast cells. J Exp Med. 2007; 204(2):253-8. doi: 10.1084/jem.20062211. PMCID: PMC2118732.
11. Harden J L, Krueger J G, Bowcock A M. The immunogenetics of Psoriasis: A comprehensive review. J Autoimmun. 2015; 64:66-73. doi: 10.1016/j.jaut.2015.07.008. PMCID: PMC4628849.
12. Gittler J K, Shemer A, Suarez-Farinas M, Fuentes-Duculan J, Gulewicz K J, Wang C Q, Mitsui H, Cardinale I, de Guzman Strong C, Krueger J G, Guttman-Yassky E. Progressive activation of T(H)2/T(H)22 cytokines and selective epidermal proteins characterizes acute and chronic atopic dermatitis. J Allergy Clin Immunol. 2012; 130(6):1344-54. doi: 10.1016/j.jaci.2012.07.012. PMCID: PMC3991245.
13. Martin D A, Towne J E, Kricorian G, Klekotka P, Gudjonsson J E, Krueger J G, Russell C B. The emerging role of IL-17 in the pathogenesis of psoriasis: preclinical and clinical findings. J Invest Dermatol. 2013; 133(1):17-26. doi: 10.1038/jid.2012.194. PMCID: PMC3568997.
14. Wilsmann-Theis D, Hagemann T, Jordan J, Bieber T, Novak N. Facing psoriasis and atopic dermatitis: are there more similarities or more differences? Eur J Dermatol. 2008; 18(2):172-80. doi: 10.1684/ejd.2008.0357.
15. Nograles K E, Zaba L C, Shemer A, Fuentes-Duculan J, Cardinale I, Kikuchi T, Ramon M, Bergman R, Krueger J G, Guttman-Yassky E. IL-22-producing “T22” T cells account for upregulated IL-22 in atopic dermatitis despite reduced IL-17-producing TH17 T cells. J Allergy Clin Immunol. 2009; 123(6):1244-52 e2. doi: 10.1016/j.jaci.2009.03.041. PMCID: PMC2874584.
16. Teraki Y, Sakurai A, Izaki S. IL-13/IL-22-coproducing T cells, a novel subset, are increased in atopic dermatitis. J Allergy Clin Immunol. 2013; 132(4):971-4. doi: 10.1016/j.jaci.2013.07.029.
17. Czarnowicki T, Gonzalez J, Shemer A, Malajian D, Xu H, Zheng X, Khattri S, Gilleaudeau P, Sullivan-Whalen M, Suarez-Farinas M, Krueger J G, Guttman-Yassky E. Severe atopic dermatitis is characterized by selective expansion of circulating TH2/TC2 and TH22/TC22, but not TH17/TC17, cells within the skin-homing T-cell population. J Allergy Clin Immunol. 2015; 136(1):104-15 e7. doi: 10.1016/j.jaci.2015.01.020.
18. Leyva-Castillo J M, Yoon J, Geha R S. IL-22 promotes allergic airway inflammation in epicutaneously sensitized mice. J Allergy Clin Immunol. 2018. doi: 10.1016/j.jaci.2018.05.032. PMCID: PMC6298864.
19. Glocova I, Bruck J, Geisel J, Muller-Hermelink E, Widmaier K, Yazdi A S, Rocken M, Ghoreschi K. Induction of skin-pathogenic Th22 cells by epicutaneous allergen exposure. Journal of dermatological science. 2017; 87(3):268-77. doi: 10.1016/j.jdermsci.2017.06.006.
20. Renert-Yuval Y, Guttman-Yassky E. Systemic therapies in atopic dermatitis: The pipeline. Clin Dermatol. 2017; 35(4):387-97. doi: 10.1016/j.clindermatol.2017.03.012.
21. Czarnowicki T, He H, Krueger J G, Guttman-Yassky E. Atopic dermatitis endotypes and implications for targeted therapeutics. J Allergy Clin Immunol. 2019; 143(1):1-11. doi: 10.1016/j.jaci.2018.10.032.
22. Tsoi L C, Rodriguez E, Degenhardt F, Baurecht H, Wehkamp U, Volks N, Szymczak S, Swindell W R, Sarkar M K, Raja K, Shao S, Patrick M, Gao Y, Uppala R, Perez White B E, Getsios S, Harms P W, Maverakis E, Elder J T, Franke A, Gudjonsson J E, Weidinger S. Atopic Dermatitis Is an IL-13-Dominant Disease with Greater Molecular Heterogeneity Compared to Psoriasis. J Invest Dermatol. 2019; 139(7):1480-9. doi: 10.1016/j.jid.2018.12.018. PMCID: PMC6711380.
23. Croft M. Costimulation of T cells by OX40, 4-1BB, and CD27. Cytokine Growth Factor Rev. 2003; 14(3-4):265-73.
24. Croft M. The role of TNF superfamily members in T-cell function and diseases. Nat Rev Immunol. 2009; 9(4): 271-85. doi: nri2526 [pii] 10.1038/nri2526.
25. Shaikh R B, Santee S, Granger S W, Butrovich K, Cheung T, Kronenberg M, Cheroutre H, Ware C F. Constitutive expression of LIGHT on T cells leads to lymphocyte activation, inflammation, and tissue destruction. J Immunol. 2001; 167(11):6330-7.
26. Wang J, Lo J C, Foster A, Yu P, Chen H M, Wang Y, Tamada K, Chen L, Fu Y X. The regulation of T cell homeostasis and autoimmunity by T cell-derived LIGHT. J Clin Invest. 2001; 108(12):1771-80.
27. Lo J C, Wang Y, Tumanov A V, Bamji M, Yao Z, Reardon C A, Getz G S, Fu Y X. Lymphotoxin beta receptor-dependent control of lipid homeostasis. Science. 2007; 316(5822):285-8.
28. Wang J, Anders R A, Wang Y, Turner J R, Abraham C, Pfeffer K, Fu Y X. The critical role of LIGHT in promoting intestinal inflammation and Crohn's disease. J Immunol. 2005; 174(12):8173-82.
29. Mauri D N, Ebner R, Montgomery R I, Kochel K D, Cheung T C, Yu G L, Ruben S, Murphy M, Eisenberg R J, Cohen G H, Spear P G, Ware C F. LIGHT, a new member of the TNF superfamily, and lymphotoxin alpha are ligands for herpesvirus entry mediator. Immunity. 1998; 8(1):21-30.
30. Ware C F. Targeting lymphocyte activation through the lymphotoxin and LIGHT pathways. Immunol Rev. 2008; 223:186-201. doi: IMR629 [pii]10.1111/j.1600-065X.2008.00629.x.
31. Croft M, Siegel R M. Beyond TNF: TNF superfamily cytokines as targets for the treatment of rheumatic diseases. Nat Rev Rheumatol. 2017; 13(4):217-33. doi: 10.1038/nrrheum.2017.22.
32. Steinberg M W, Shui J W, Ware C F, Kronenberg M. Regulating the mucosal immune system: the contrasting roles of LIGHT, HVEM, and their various partners. Semin Immunopathol. 2009; 31(2):207-21. doi: 10.1007/s00281-009-0157-4. PMCID: 2766922.
33. Ando T, Matsumoto K, Namiranian S, Yamashita H, Glatthorn H, Kimura M, Dolan B R, Lee J J, Galli S J, Kawakami Y, Jamora C, Kawakami T. Mast cells are required for full expression of allergen/SEB-induced skin inflammation. J Invest Dermatol. 2013; 133(12):2695-705. doi: 10.1038/jid.2013.250. PMCID: 3830701.
34. Ando T, Xiao W, Gao P, Namiranian S, Matsumoto K, Tomimori Y, Hong H, Yamashita H, Kimura M, Kashiwakura J, Hata T R, Izuhara K, Gurish M F, Roers A, Rafaels N M, Barnes K C, Jamora C, Kawakami Y, Kawakami T. Critical role for mast cell StatS activity in skin inflammation. Cell reports. 2014; 6(2):366-76. doi: 10.1016/j.celrep.2013.12.029. PMCID: 4329986.
35. Kawakami Y, Yumoto K, Kawakami T. An improved mouse model of atopic dermatitis and suppression of skin lesions by an inhibitor of Tec family kinases. Allergology international: official journal of the Japanese Society of Allergology. 2007; 56(4):403-9. doi: 10.2332/allergolint.O-07-486.
36. Arima K, Ohta S, Takagi A, Shiraishi H, Masuoka M, Ontsuka K, Suto H, Suzuki S, Yamamoto K, Ogawa M, Simmons O, Yamaguchi Y, Toda S, Aihara M, Conway S J, Ikeda S, Izuhara K. Periostin contributes to epidermal hyperplasia in psoriasis common to atopic dermatitis. Allergology international: official journal of the Japanese Society of Allergology. 2015; 64(1):41-8. doi: 10.1016/j.alit.2014.06.001. PMCID: 4793727.
37. El Malki K, Karbach S H, Huppert J, Zayoud M, Reissig S, Schuler R, Nikolaev A, Karram K, Munzel T, Kuhlmann C R, Luhmann H J, von Stebut E, Wortge S, Kurschus F C, Waisman A. An alternative pathway of imiquimod-induced psoriasis-like skin inflammation in the absence of interleukin-17 receptor a signaling J Invest Dermatol. 2013; 133 (2):441-51. doi: 10.1038/jid.2012.318.
38. Van Belle A B, de Heusch M, Lemaire M M, Hendrickx E, Warnier G, Dunussi-Joannopoulos K, Fouser L A, Renauld J C, Dumoutier L. IL-22 is required for imiquimod-induced psoriasiform skin inflammation in mice. J Immunol. 2012; 188(1):462-9. doi: 10.4049/jimmunol.1102224.
39. Yoshiki R, Kabashima K, Honda T, Nakamizo S, Sawada Y, Sugita K, Yoshioka H, Ohmori S, Malissen B, Tokura Y, Nakamura M. IL-23 from Langerhans cells is required for the development of imiquimod-induced psoriasis-like dermatitis by induction of IL-17A-producing gammadelta T cells. J Invest Dermatol. 2014; 134(7):1912-21. doi: 10.1038/jid.2014.98.
40. Meylan F, Richard A C, Siegel R M. TL1A and DR3, a TNF family ligand-receptor pair that promotes lymphocyte costimulation, mucosal hyperplasia, and autoimmune inflammation. Immunological reviews. 2011; 244(1):188-96. doi: 10.1111/j.1600-065X.2011.01068.x.
41. da Silva Antunes R, Madge L, Soroosh P, Tocker J, Croft M. The TNF Family Molecules LIGHT and Lymphotoxin alphabeta Induce a Distinct Steroid-Resistant Inflammatory Phenotype in Human Lung Epithelial Cells. J Immunol. 2015; 195(5):2429-41. doi: 10.4049/jimmunol.1500356. PMCID: 4546856.
42. da Silva Antunes R, Mehta A K, Madge L, Tocker J, Croft M. TNFSF14 (LIGHT) Exhibits Inflammatory Activities in Lung Fibroblasts Complementary to IL-13 and TGF-beta. Front Immunol. 2018; 9:576. doi: 10.3389/fimmu.2018.00576. PMCID: PMC5868327.
43. Al-Lamki R S, Wang J, Tolkovsky A M, Bradley J A, Griffin J L, Thiru S, Wang E C, Bolton E, Min W, Moore P, Pober J S, Bradley J R. TL1A both promotes and protects from renal inflammation and injury. J Am Soc Nephrol. 2008; 19(5):953-60. doi: ASN.2007060706 [pii]10.1681/ASN.2007060706. PMCID: 2386725.
44. Shih D Q, Zheng L, Zhang X, Zhang H, Kanazawa Y, Ichikawa R, Wallace K L, Chen J, Pothoulakis C, Koon H W, Targan S R. Inhibition of a novel fibrogenic factor Tl1a reverses established colonic fibrosis. Mucosal Immunol. 2014; 7(6):1492-503. doi: 10.1038/mi.2014.37. PMCID: 4205266.
45. Ma Z, Wang B, Wang M, Sun X, Tang Y, Li M, Li F, Li X. TL1A increased IL-6 production on fibroblast-like synoviocytes by preferentially activating TNF receptor 2 in rheumatoid arthritis. Cytokine. 2016; 83:92-8. doi: 10.1016/j.cyto.2016.04.005.
46. Kotani H, Masuda K, Tamagawa-Mineoka R, Nomiyama T, Soga F, Nin M, Asai J, Kishimoto S, Katoh N. Increased plasma LIGHT levels in patients with atopic dermatitis. Clinical and experimental immunology. 2012; 168(3):318-24. doi: 10.1111/j.1365-2249.2012.04576.x. PMCID: 3390484.
47. Morimura S, Sugaya M, Kai H, Kato T, Miyagaki T, Ohmatsu H, Kagami S, Asano Y, Mitsui H, Tada Y, Kadono T, Sato S. High levels of LIGHT and low levels of soluble herpesvirus entry mediator in sera of patients with atopic dermatitis. Clin Exp Dermatol. 2012; 37(2):181-2. doi: 10.1111/j.1365-2230.2011.04079.x.
48. Jung H W, La S J, Kim J Y, Heo S K, Wang S, Kim K K, Lee K M, Cho H R, Lee H W, Kwon B, Kim B S, Kwon B S. High levels of soluble herpes virus entry mediator in sera of patients with allergic and autoimmune diseases. Exp Mol Med. 2003; 35(6):501-8.
49. Li L, Fu L, Lu Y, Wang W, Liu H, Li F, Chen T. TNF-like ligand 1A is associated with the pathogenesis of psoriasis vulgaris and contributes to IL-17 production in PBMCs. Arch Dermatol Res. 2014; 306(10):927-32. doi: 10.1007/s00403-014-1497-z.
50. Pedersen A E, Schmidt E G, Sorensen J F, Faber C, Nielsen B S, Holmstrom K, Omland S H, Tougaard P, Skov S, Bang B. Secretion, blood levels and cutaneous expression of TL1A in psoriasis patients. APMIS. 2015; 123(7):547-55. doi: 10.1111/apm.12385.
51. Sun L D, Xiao F L, Li Y, Zhou W M, Tang H Y, Tang X F, Zhang H, Schaarschmidt H, Zuo X B, Foelster-Holst R, He S M, Shi M, Liu Q, Lv Y M, Chen X L, Zhu K J, Guo Y F, Hu D Y, Li M, Li M, Zhang Y H, Zhang X, Tang J P, Guo B R, Wang H, Liu Y, Zou X Y, Zhou F S, Liu X Y, Chen G, Ma L, Zhang S M, Jiang A P, Zheng X D, Gao X H, Li P, Tu C X, Yin X Y, Han X P, Ren Y Q, Song S P, Lu Z Y, Zhang X L, Cui Y, Chang J, Gao M, Luo X Y, Wang P G, Dai X, Su W, Li H, Shen C P, Liu S X, Feng X B, Yang C J, Lin G S, Wang Z X, Huang J Q, Fan X, Wang Y, Bao Y X, Yang S, Liu J J, Franke A, Weidinger S, Yao Z R, Zhang X J. Genome-wide association study identifies two new susceptibility loci for atopic dermatitis in the Chinese Han population. Nat Genet. 2011; 43(7):690-4. doi: 10.1038/ng.851.
52. Ellinghaus D, Baurecht H, Esparza-Gordillo J, Rodriguez E, Matanovic A, Marenholz I, Hubner N, Schaarschmidt H, Novak N, Michel S, Maintz L, Werfel T, Meyer-Hoffert U, Hotze M, Prokisch H, Heim K, Herder C, Hirota T, Tamari M, Kubo M, Takahashi A, Nakamura Y, Tsoi L C, Stuart P, Elder J T, Sun L, Zuo X, Yang S, Zhang X, Hoffmann P, Nothen M M, Folster-Holst R, Winkelmann J, Illig T, Boehm B O, Duerr R H, Buning C, Brand S, Glas J, McAleer M A, Fahy C M, Kabesch M, Brown S, McLean W H, Irvine A D, Schreiber S, Lee Y A, Franke A, Weidinger S. High-density genotyping study identifies four new susceptibility loci for atopic dermatitis. Nat Genet. 2013; 45(7):808-12. doi: 10.1038/ng.2642. PMCID: PMC3797441.
53. He H, Suryawanshi H, Morozov P, Gay-Mimbrera J, Del Duca E, Kim H J, Kameyama N, Estrada Y, Der E, Krueger J G, Ruano J, Tuschl T, Guttman-Yassky E. Single-cell transcriptome analysis of human skin identifies novel fibroblast subpopulation and enrichment of immune subsets in atopic dermatitis. J Allergy Clin Immunol. 2020. doi: 10.1016/j.jaci.2020.01.042.
54. Shiraishi H, Masuoka M, Ohta S, Suzuki S, Arima K, Taniguchi K, Aoki S, Toda S, Yoshimoto T, Inagaki N, Conway S J, Narisawa Y, Izuhara K. Periostin contributes to the pathogenesis of atopic dermatitis by inducing TSLP production from keratinocytes. Allergology international: official journal of the Japanese Society of Allergology. 2012; 61(4):563-72. doi: 10.2332/allergolint.10-OA-0297.
55. Balato A, Lembo S, Mattii M, Schiattarella M, Marino R, De Paulis A, Balato N, Ayala F. IL-33 is secreted by psoriatic keratinocytes and induces pro-inflammatory cytokines via keratinocyte and mast cell activation. Exp Dermatol. 2012; 21(11):892-4. doi: 10.1111/exd.12027.
56. Savinko T, Matikainen S, Saarialho-Kere U, Lehto M, Wang G, Lehtimaki S, Karisola P, Reunala T, Wolff H, Lauerma A, Alenius H. IL-33 and ST2 in atopic dermatitis: expression profiles and modulation by triggering factors. J Invest Dermatol. 2012; 132(5):1392-400. doi: 10.1038/jid.2011.446.
57. Aktar M K, Kido-Nakahara M, Fume M, Nakahara T. Mutual upregulation of endothelin-1 and I L-25 in atopic dermatitis. Allergy. 2015; 70(7):846-54. doi: 10.1111/a11.12633.
58. Szegedi K, Lutter R, Res P C, Bos J D, Luiten R M, Kezic S, Middelkamp-Hup M A. Cytokine profiles in interstitial fluid from chronic atopic dermatitis skin. Journal of the European Academy of Dermatology and Venereology: JEADV. 2015; 29(11):2136-44. doi: 10.1111/jdv.13160.
59. Balato A, Di Caprio R, Canta L, Mattii M, Lembo S, Raimondo A, Schiattarella M, Balato N, Ayala F. IL-33 is regulated by TNF-alpha in normal and psoriatic skin. Arch Dermatol Res. 2014; 306(3):299-304. doi: 10.1007/s00403-014-1447-9.
60. Mitsui A, Tada Y, Takahashi T, Shibata S, Kamata M, Miyagaki T, Fujita H, Sugaya M, Kadono T, Sato S, Asano Y. Serum IL-33 levels are increased in patients with psoriasis. Clin Exp Dermatol. 2016; 41(2):183-9. doi: 10.1111/ced.12670.
61. Bissonnette R, Fuentes-Duculan J, Mashiko S, Li X, Bonifacio K M, Cueto I, Suarez-Farinas M, Maari C, Bolduc C, Nigen S, Sarfati M, Krueger J G. Palmoplantar pustular psoriasis (PPPP) is characterized by activation of the IL-17A pathway. Journal of dermatological science. 2017; 85(1):20-6. doi: 10.1016/jjdermsci.2016.09.019.
62. Raychaudhuri S P, Jiang W Y, Farber E M, Schall T J, Ruff M R, Pert C B. Upregulation of RANTES in psoriatic keratinocytes: a possible pathogenic mechanism for psoriasis. Acta Derm Venereol. 1999; 79(1):9-11.
63. Patel D D, Kuchroo V K. Th17 Cell Pathway in Human Immunity: Lessons from Genetics and Therapeutic Interventions. Immunity. 2015; 43(6):1040-51. doi: 10.1016/j.immuni.2015.12.003.
64. Campa M, Mansouri B, Warren R, Menter A. A Review of Biologic Therapies Targeting IL-23 and IL-17 for Use in Moderate-to-Severe Plaque Psoriasis. Dermatology and therapy. 2016; 6(1):1-12. doi: 10.1007/s13555-015-0092-3. PMCID: 4799039.
65. Buzney C D, Gottlieb A B, Rosmarin D. Asthma and Atopic Dermatitis: A Review of Targeted Inhibition of Interleukin-4 and Interleukin-13 As Therapy for Atopic Disease. Journal of drugs in dermatology: JDD. 2016; 15(2):165-71.
66. Roesner L M, Werfel T, Heratizadeh A. The adaptive immune system in atopic dermatitis and implications on therapy. Expert review of clinical immunology. 2016:1-10. doi: 10.1586/1744666X.2016.1165093.
67. Guttman-Yassky E, Dhingra N, Leung D Y. New era of biologic therapeutics in atopic dermatitis. Expert OpinBiol Ther. 2013; 13(4):549-61. doi: 10.1517/14712598.2013.758708. PMCID: 3819721.
68. Mansouri Y, Guttman-Yassky E. Immune Pathways in Atopic Dermatitis, and Definition of Biomarkers through Broad and Targeted Therapeutics. Journal of clinical medicine. 2015; 4(5):858-73. doi: 10.3390/jcm4050858. PMCID: 4470203.
69. Mu Z, Zhao Y, Liu X, Chang C, Zhang J. Molecular biology of atopic dermatitis. Clinical reviews in allergy & immunology. 2014; 47(2):193-218. doi: 10.10071s12016-014-8415-1.
70. Bissonnette R, Suarez-Farinas M, Li X, Bonifacio K M, Brodmerkel C, Fuentes-Duculan J, Krueger J G. Based on Molecular Profiling of Gene Expression, Palmoplantar Pustulosis and Palmoplantar Pustular Psoriasis Are Highly Related Diseases that Appear to Be Distinct from Psoriasis Vulgaris. PloS one. 2016; 11(5):e0155215. doi: 10.1371/journal.pone.0155215. PMCID: PMC4859542.
71. Ziegler S F. The role of thymic stromal lymphopoietin (TSLP) in allergic disorders. Current opinion in immunology. 2010; 22(6):795-9. doi: 10.1016/j.coi.2010.10.020. PMCID: 3032269.
72. Deleuran M, Hvid M, Kemp K, Christensen G B, Deleuran B, Vestergaard C. IL-25 induces both inflammation and skin barrier dysfunction in atopic dermatitis. Chemical immunology and allergy. 2012; 96:45-9. doi: 10.1159/000331871.
73. Hvid M, Vestergaard C, Kemp K, Christensen G B, Deleuran B, Deleuran M. IL-25 in atopic dermatitis: a possible link between inflammation and skin barrier dysfunction? J Invest Dermatol. 2011; 131(1):150-7. doi: 10.1038/jid.2010.277.
74. Salimi M, Barlow J L, Saunders S P, Xue L, Gutowska-Owsiak D, Wang X, Huang L C, Johnson D, Scanlon S T, McKenzie A N, Fallon P G, Ogg G S. A role for IL-25 and IL-33-driven type-2 innate lymphoid cells in atopic dermatitis. J Exp Med. 2013; 210(13):2939-50. doi: 10.1084/jem.20130351. PMCID: PMC3865470.
75. Du H Y, Fu H Y, Li D N, Qiao Y, Wang Q W, Liu W. The Expression and Regulation of Interleukin-33 in Human Epidermal Keratinocytes: A New Mediator of Atopic Dermatitis and Its Possible Signaling Pathway. J Interferon Cytokine Res. 2016; 36(9):552-62. doi: 10.1089/jir.2015.0159.
76. Yu B, Koga T, Urabe K, Moroi Y, Maeda S, Yanagihara Y, Fume M. Differential regulation of thymus- and activation-regulated chemokine induced by IL-4, IL-13, TNF-alpha and IFN-gamma in human keratinocyte and fibroblast. Journal of dermatological science. 2002; 30(1):29-36.
77. Kakinuma T, Nakamura K, Wakugawa M, Yano S, Saeki H, Torii H, Komine M, Asahina A, Tamaki K. IL-4, but not IL-13, modulates TARC (thymus and activation-regulated chemokine)/CCL17 and IP-10 (interferon-induced protein of 10 kDA)/CXCL10 release by TNF-alpha and IFN-gamma in HaCaT cell line. Cytokine. 2002; 20(1):1-6.
78. Eyerich S, Eyerich K, Pennino D, Carbone T, Nasorri F, Pallotta S, Cianfarani F, Odorisio T, Traidl-Hoffmann C, Behrendt H, Durham S R, Schmidt-Weber C B, Cavani A. Th22 cells represent a distinct human T cell subset involved in epidermal immunity and remodeling. J Clin Invest. 2009; 119(12):3573-85. doi: 10.1172/JCI40202. PMCID: 2786807.
79. Fujita H. The role of IL-22 and Th22 cells in human skin diseases. Journal of dermatological science. 2013; 72(1):3-8. doi: 10.1016/jjdermsci.2013.04.028.
80. Hayashida S, Uchi H, Takeuchi S, Esaki H, Moroi Y, Fume M. Significant correlation of serum I L-22 levels with CCL17 levels in atopic dermatitis. Journal of dermatological science. 2011; 61(1):78-9. doi: 10.1016/j.jdermsci.2010.08.013.
81. Lou H, Lu J, Choi E B, Oh M H, Jeong M, Barmettler S, Zhu Z, Zheng T. Expression of IL-22 in the Skin Causes Th2-Biased Immunity, Epidermal Barrier Dysfunction, and Pruritus via Stimulating Epithelial Th2 Cytokines and the GRP Pathway. J Immunol. 2017; 198(7):2543-55. doi: 10.4049/jimmunol.1600126. PMCID: PMC5360537.
82. Sa S M, Valdez P A, Wu J, Jung K, Zhong F, Hall L, Kasman I, Winer J, Modrusan Z, Danilenko D M, Ouyang W. The effects of IL-20 subfamily cytokines on reconstituted human epidermis suggest potential roles in cutaneous innate defense and pathogenic adaptive immunity in psoriasis. J Immunol. 2007; 178(4):2229-40.
83. Wolk K, Witte E, Warszawska K, Schulze-Tanzil G, Witte K, Philipp S, Kunz S, Docke W D, Asadullah K, Volk H D, Sterry W, Sabat R. The Th17 cytokine IL-22 induces IL-20 production in keratinocytes: a novel immunological cascade with potential relevance in psoriasis. Eur J Immunol. 2009; 39(12):3570-81. doi: 10.1002/eji.200939687.
84. Jang M, Kim H, Kim Y, Choi J, Jeon J, Hwang Y, Kang J S, Lee W J. The crucial role of IL-22 and its receptor in thymus and activation regulated chemokine production and T-cell migration by house dust mite extract. Exp Dermatol. 2016; 25(8):598-603. doi: 10.1111/exd.12988.
85. Tohyama M, Hanakawa Y, Shirakata Y, Dai X, Yang L, Hirakawa S, Tokumaru S, Okazaki H, Sayama K, Hashimoto K. IL-17 and IL-22 mediate IL-20 subfamily cytokine production in cultured keratinocytes via increased IL-22 receptor expression. Eur J Immunol. 2009; 39(10):2779-88. doi: 10.1002/eji.200939473.
86. Bao L, Zhang H, Mohan G C, Shen K, Chan L S. Differential expression of inflammation-related genes in IL-4 transgenic mice before and after the onset of atopic dermatitis skin lesions. Mol Cell Probes. 2016; 30(1):30-8. doi: 10.1016/j.mcp.2015.11.001.
87. Kunz S, Wolk K, Witte E, Witte K, Doecke W D, Volk H D, Sterry W, Asadullah K, Sabat R. Interleukin (IL)-19, IL-20 and IL-24 are produced by and act on keratinocytes and are distinct from classical ILs. Exp Dermatol. 2006; 15(12):991-1004. doi: 10.1111/j.1600-0625.2006.00516.x.
88. He M, Liang P. IL-24 transgenic mice: in vivo evidence of overlapping functions for IL-20, IL-22, and IL-24 in the epidermis. J Immunol. 2010; 184(4):1793-8. doi: 10.4049/jimmunol.0901829.
89. Niehues H, Schalkwijk J, van Vlijmen-Willems I, Rodijk-Olthuis D, van Rossum M M, Wladykowski E, Brandner J M, van den Bogaard E H J, Zeeuwen P. Epidermal equivalents of filaggrin null keratinocytes do not show impaired skin barrier function. J Allergy Clin Immunol. 2017; 139(6):1979-81 e13. doi: 10.1016/j.jaci.2016.09.016.
90. Sayedyahossein S, Rudkouskaya A, Leclerc V, Dagnino L. Integrin-Linked Kinase Is Indispensable for Keratinocyte Differentiation and Epidermal Barrier Function. J Invest Dermatol. 2016; 136(2):425-35. doi: 10.1016/j.jid.2015.10.056.
91. Sidler D, Wu P, Herro R, Claus M, Wolf D, Kawakami Y, Kawakami T, Burkly L, Croft M. TWEAK mediates inflammation in experimental atopic dermatitis and psoriasis. Nature communications. 2017; 8:15395. doi: 10.1038/ncomms15395. PMCID: PMC5493595.
92. Malhotra N, Yoon J, Leyva-Castillo J M, Galand C, Archer N, Miller L S, Geha R S. IL-22 derived from gammadelta T cells restricts Staphylococcus aureus infection of mechanically injured skin. J Allergy Clin Immunol. 2016; 138(4):1098-107 e3. doi: 10.1016/j.jaci.2016.07.001. PMCID: PMC5056816.
93. Cairo C, Arabito E, Landi F, Casati A, Brunetti E, Mancino G, Galli E. Analysis of circulating gammadelta T cells in children affected by IgE-associated and non-IgE-associated allergic atopic eczema/dermatitis syndrome. Clinical and experimental immunology. 2005; 141(1):116-21. doi: 10.1111/j.1365-2249.2005.02813.x. PMCID: PMC1809419.
94. Katsuta M, Takigawa Y, Kimishima M, Inaoka M, Takahashi R, Shiohara T. N K cells and gamma delta+ T cells are phenotypically and functionally defective due to preferential apoptosis in patients with atopic dermatitis. J Immunol. 2006; 176(12):7736-44.
95. Sulcova J, Maddaluno L, Meyer M, Werner S. Accumulation and activation of epidermal gammadelta T cells in a mouse model of chronic dermatitis is not required for the inflammatory phenotype. Eur J Immunol. 2015; 45(9):2517-28. doi: 10.1002/eji.201545675.
96. Marsters S A, Ayres T M, Skubatch M, Gray C L, Rothe M, Ashkenazi A. Herpesvirus entry mediator, a member of the tumor necrosis factor receptor (TNFR) family, interacts with members of the TNFR-associated factor family and activates the transcription factors NF-kappaB and AP-1. J Biol Chem. 1997; 272(22): 14029-32.
97. Dejardin E, Droin N M, Delhase M, Haas E, Cao Y, Makris C, Li Z W, Karin M, Ware C F, Green D R. The lymphotoxin-beta receptor induces different patterns of gene expression via two NF-kappaB pathways. Immunity. 2002; 17(4):525-35.
98. Richard A C, Ferdinand J R, Meylan F, Hayes E T, Gabay O, Siegel R M. The TNF-family cytokine TL1A: from lymphocyte costimulator to disease co-conspirator. J Leukoc Biol. 2015; 98(3):333-45. doi: 10.1189/jlb.3R10315-095R. PMCID: 4763597.
99. Muller J R, Siebenlist U. Lymphotoxin beta receptor induces sequential activation of distinct NF-kappa B factors via separate signaling pathways. J Biol Chem. 2003; 278(14):12006-12.
100. Hauer J, Puschner S, Ramakrishnan P, Simon U, Bongers M, Federle C, Engelmann H. TNF receptor (TNFR)-associated factor (TRAF) 3 serves as an inhibitor of TRAF2/5-mediated activation of the noncanonical NF-kappaB pathway by TRAF-binding TNFRs. Proc Natl Acad Sci USA. 2005; 102(8):2874-9.
101. Zarnegar B J, Wang Y, Mahoney D J, Dempsey P W, Cheung H H, He J, Shiba T, Yang X, Yeh W C, Mak T W, Korneluk R G, Cheng G. Noncanonical NF-kappaB activation requires coordinated assembly of a regulatory complex of the adaptors cIAP1, cIAP2, TRAF2 and TRAF3 and the kinase NIK. Nature immunology. 2008; 9(12):1371-8. doi: 10.1038/ni.1676. PMCID: 2676931.
102. Soroosh P, Doherty T A, So T, Mehta A K, Khorram N, Norris P S, Scheu S, Pfeffer K, Ware C, Croft M. Herpesvirus entry mediator (TNFRSF14) regulates the persistence of T helper memory cell populations. Journal of Experimental Medicine. 2011; 208(4):797-809. doi: 10.1084/jem.20101562. PMCID: 3135347.
103. So T, Croft M. Regulation of PI-3-Kinase and Akt Signaling in T Lymphocytes and Other Cells by TNFR Family Molecules. Frontiers in immunology. 2013; 4:139. doi: 10.3389/fimmu.2013.00139. PMCID: 3675380.
104. Schreiber T H, Podack E R. Immunobiology of TNFSF15 and TNFRSF25. Immunol Res. 2013; 57(1-3):3-11. doi: 10.10071s12026-013-8465-0.
105. Lee S H, Kim E J, Suk K, Kim I S, Lee W H. TL1A induces the expression of TGF-beta-inducible gene h3 (betaig-h3) through PKC, PI3K, and ERK in THP-1 cells. Cell Immunol. 2010; 266(1):61-6. doi: 10.1016/j.cellimm.2010.08.013.
106. Lee H C, Ziegler S F. Inducible expression of the proallergic cytokine thymic stromal lymphopoietin in airway epithelial cells is controlled by NFkappaB. Proc Natl Acad Sci USA. 2007; 104(3):914-9. doi: 10.1073/pnas.0607305104. PMCID: 1783414.
107. Vu A T, Chen X, Xie Y, Kamijo S, Ushio H, Kawasaki J, Hara M, Ikeda S, Okumura K, Ogawa H, Takai T. Extracellular double-stranded RNA induces TSLP via an endosomal acidification- and NF-kappaB-dependent pathway in human keratinocytes. The Journal of investigative dermatology. 2011; 131(11):2205-12. doi: 10.1038/jid.2011.185.
108. Rogers P R, Song J, Gramaglia I, Killeen N, Croft M. OX40 promotes Bcl-xL and Bcl-2 expression and is essential for long-term survival of CD4 T cells. Immunity. 2001; 15:445-55.
109. Song J, Salek-Ardakani S, Rogers P R, Cheng M, Van Parijs L, Croft M. The costimulation-regulated duration of PKB activation controls T cell longevity. Nature immunology. 2004; 5(2):150-8.
110. Song J, So T, Cheng M, Tang X, Croft M. Sustained survivin expression from OX40 costimulatory signals drives T cell clonal expansion. Immunity. 2005; 22:621-31.
111. So T, Song J, Sugie K, Altman A, Croft M. Signals from OX40 regulate nuclear factor of activated T cells cl and T cell helper 2 lineage commitment. Proc Natl Acad Sci USA. 2006; 103(10):3740-5.
112. Song J, Salek-Ardakani S, So T, Croft M. The kinases aurora B and mTOR regulate the G1-S cell cycle progression of T lymphocytes. Nat Immunol. 2007; 8(1):64-73.
113. Song J, So T, Croft M. Activation of NF-{kappa}B1 by OX40 Contributes to Antigen-Driven T Cell Expansion and Survival. J Immunol. 2008; 180(11):7240-8. doi: 180/11/7240 [pii]. PMCID: 2410213.
114. Banno T, Gazel A, Blumenberg M. Pathway-specific profiling identifies the NF-kappa B-dependent tumor necrosis factor alpha-regulated genes in epidermal keratinocytes. J Biol Chem. 2005; 280(19):18973-80. doi: 10.1074/jbc.M411758200.
115. Nadiminty N, Chun J Y, Hu Y, Dutt S, Lin X, Gao A C. LIGHT, a member of the TNF superfamily, activates Stat3 mediated by NIK pathway. Biochem Biophys Res Commun. 2007; 359(2):379-84. doi: 10.1016/j.bbrc.2007.05.119. PMCID: 2062522.
116. Su W B, Chang Y H, Lin W W, Hsieh S L. Differential regulation of interleukin-8 gene transcription by death receptor 3 (DR3) and type I TNF receptor (TNFRI). Exp Cell Res. 2006; 312(3):266-77. doi: 10.1016/j.yexcr.2005.10.015.
117. Yeh D Y, Wu C C, Chin Y P, Lu C J, Wang Y H, Chen M C. Mechanisms of human lymphotoxin beta receptor activation on upregulation of CCL5/RANTES production. Int Immunopharmacol. 2015; 28(1):220-9. doi: 10.1016/j.intimp.2015.06.010.
118. Wu N L, Huang D Y, Tsou FIN, Lin Y C, Lin W W. Syk mediates IL-17-induced CCL20 expression by targeting Act1-dependent K63-linked ubiquitination of TRAF6. J Invest Dermatol. 2015; 135(2):490-8. doi: 10.1038/jid.2014.383.
119. Zhu L, Wu Y, Wei H, Xing X, Zhan N, Xiong H, Peng B. IL-17R activation of human periodontal ligament fibroblasts induces IL-23 p19 production: Differential involvement of NF-kappaB versus JNK/AP-1 pathways. Mol Immunol. 2011; 48(4):647-56. doi: 10.1016/j.molimm.2010.11.008.
120. Dong Z, Yang Y, Zhang T, Li Y, Kang Q, Lei W, Cao Y, Niu X, Wang D, Tai W. siRNA-Actl inhibits the function of IL-17 on lung fibroblasts via the NF-kappaB pathway. Respiration. 2013; 86(4):332-40. doi: 10.1159/000348403.
121. Zhu S, Pan W, Shi P, Gao H, Zhao F, Song X, Liu Y, Zhao L, Li X, Shi Y, Qian Y. Modulation of experimental autoimmune encephalomyelitis through TRAF3-mediated suppression of interleukin 17 receptor signaling. J Exp Med. 2010; 207(12):2647-62. doi: 10.1084/jem.20100703. PMCID: PMC2989772.
122. Hwang S Y, Kim J Y, Kim K W, Park M K, Moon Y, Kim W U, Kim H Y. IL-17 induces production of IL-6 and IL-8 in rheumatoid arthritis synovial fibroblasts via NF-kappaB- and PI3-kinase/Akt-dependent pathways. Arthritis Res Ther. 2004; 6(2):R120-8. doi: 10.1186/ar1038. PMCID: PMC400429.
123. Hattori T, Ogura N, Akutsu M, Kawashima M, Watanabe S, Ito K, Kondoh T. Gene Expression Profiling of IL-17A-Treated Synovial Fibroblasts from the Human Temporomandibular Joint. Mediators Inflamm. 2015; 2015:436067. doi: 10.1155/2015/436067. PMCID: PMC4709758.
124. Jinnin M, Ihn H, Asano Y, Yamane K, Trojanowska M, Tamaki K. Upregulation of tenascin-C expression by IL-13 in human dermal fibroblasts via the phosphoinositide 3-kinase/Akt and the protein kinase C signaling pathways. J Invest Dermatol. 2006; 126(3): 551-60. doi: 10.1038/sj jid.5700090.
125. Jinnin M, Ihn H, Yamane K, Tamaki K. Interleukin-13 stimulates the transcription of the human alpha2(I) collagen gene in human dermal fibroblasts. J Biol Chem. 2004; 279(40):41783-91. doi: 10.1074/jbc.M406951200.
126. Guo J, Yao H, Lin X, Xu H, Dean D, Zhu Z, Liu G, Sime P. IL-13 induces YY1 through the AKT pathway in lung fibroblasts. PloS one. 2015; 10(3):e0119039. doi: 10.1371/journal.pone.0119039. PMCID: PMC4361578.
127. Hashimoto S, Gon Y, Takeshita I, Maruoka S, Hone T. IL-4 and IL-13 induce myofibroblastic phenotype of human lung fibroblasts through c-Jun NH2-terminal kinase-dependent pathway. J Allergy Clin Immunol. 2001; 107(6):1001-8. doi: 10.1067/mai.2001.114702.
128. Zhou X, Hu H, Huynh M L, Kotaru C, Balzar S, Trudeau J B, Wenzel S E. Mechanisms of tissue inhibitor of metalloproteinase 1 augmentation by IL-13 on TGF-beta 1-stimulated primary human fibroblasts. J Allergy Clin Immunol. 2007; 119(6):1388-97. doi: 10.1016/j.jaci.2007.02.011.
129. Kim H R, Cho M L, Kim K W, Juhn J Y, Hwang S Y, Yoon C H, Park S H, Lee S H, Kim H Y. Upregulation of IL-23p19 expression in rheumatoid arthritis synovial fibroblasts by IL-17 through PI3-kinase-, NF-kappaB- and p38 MAPK-dependent signalling pathways. Rheumatology (Oxford). 2007; 46(1):57-64. doi: 10.1093/rheumatology/kel159.
130. Zhang Z, Andoh A, Yasui H, Inatomi O, Hata K, Tsujikawa T, Kitoh K, Takayanagi A, Shimizu N, Fujiyama Y. Interleukin-1 beta and tumor necrosis factor-alpha upregulate interleukin-23 subunit p19 gene expression in human colonic subepithelial myofibroblasts. International journal of molecular medicine. 2005; 15(1):79-83.
131. Berroth A, Kuhnl J, Kurschat N, Schwarz A, Stab F, Schwarz T, Wenck H, Folster-Holst R, Neufang G. Role of fibroblasts in the pathogenesis of atopic dermatitis. J Allergy Clin Immunol. 2013; 131(6):1547-54. doi: 10.1016/j.jaci.2013.02.029.
132. Ha H L, Wang H, Pisitkun P, Kim J C, Tassi I, Tang W, Morasso M I, Udey M C, Siebenlist U. IL-17 drives psoriatic inflammation via distinct, target cell-specific mechanisms. Proc Natl Acad Sci USA. 2014; 111(33):E3422-31. doi: 10.1073/pnas.1400513111. PMCID: 4143007.
133. Oyama N, Iwatsuki K, Satoh M, Akiba H, Kaneko F. Dermal fibroblasts are one of the therapeutic targets for topical application of 1alpha,25-dihydroxyvitamin D3: the possible involvement of transforming growth factor-beta induction. Br J Dermatol. 2000; 143(6):1140-8. doi: 10.1046/j.1365-2133.2000.03880.x.
134. Saiag P, Coulomb B, Lebreton C, Bell E, Dubertret L. Psoriatic fibroblasts induce hyperproliferation of normal keratinocytes in a skin equivalent model in vitro. Science. 1985; 230(4726):669-72. doi: 10.1126/science.2413549.
135. Quan C, Cho M K, Shao Y, Mianecki L E, Liao E, Perry D, Quan T. Dermal fibroblast expression of stromal cell-derived factor-1 (SDF-1) promotes epidermal keratinocyte proliferation in normal and diseased skin. Protein Cell. 2015; 6(12):890-903. doi: 10.1007/s13238-015-0198-5. PMCID: PMC4656211.
136. Guban B, Vas K, Balog Z, Manczinger M, Bebes A, Groma G, Szell M, Kemeny L, Bata-Csorgo Z. Abnormal regulation of fibronectin production by fibroblasts in psoriasis. Br J Dermatol. 2016; 174(3):533-41. doi: 10.1111/bjd.14219.
137. Donovan J, Shiwen X, Norman J, Abraham D. Platelet-derived growth factor alpha and beta receptors have overlapping functional activities towards fibroblasts. Fibrogenesis Tissue Repair. 2013; 6(1):10. doi: 10.1186/1755-1536-6-10. PMCID: 3667071.
138. Sakota Y, Ozawa Y, Yamashita H, Tanaka H, Inagaki N. Collagen gel contraction assay using human bronchial smooth muscle cells and its application for evaluation of inhibitory effect of formoterol. Biol Pharm Bull. 2014; 37(6):1014-20.
139. Beppu L Y, Anilkumar A A, Newbury R O, Dohil R, Broide D H, Aceves S S. TGF-beta1-induced phospholamban expression alters esophageal smooth muscle cell contraction in patients with eosinophilic esophagitis. J Allergy Clin Immunol. 2014; 134(5):1100-7 e4. doi: 10.1016/j.jaci.2014.04.004. PMCID: 4231011.
140. Denton C P, Khan K, Hoyles R K, Shiwen X, Leoni P, Chen Y, Eastwood M, Abraham D J. Inducible lineage-specific deletion of TbetaRII in fibroblasts defines a pivotal regulatory role during adult skin wound healing. The Journal of investigative dermatology. 2009; 129(1):194-204. doi: 10.1038/jid.2008.171.
141. Hoyles R K, Derrett-Smith E C, Khan K, Shiwen X, Howat S L, Wells A U, Abraham D J, Denton C P. An essential role for resident fibroblasts in experimental lung fibrosis is defined by lineage-specific deletion of high-affinity type II transforming growth factor beta receptor. American journal of respiratory and critical care medicine. 2011; 183(2):249-61. doi: 10.1164/rccm.201002-02790C.
142. Wang Y, Edelmayer R, Wetter J, Salte K, Gauvin D, Leys L, Paulsboe S, Su Z, Weinberg I, Namovic M, Gauld S B, Honore P, Scott V E, McGaraughty S. Monocytes/Macrophages play a pathogenic role in IL-23 mediated psoriasis-like skin inflammation. Sci Rep. 2019; 9(1):5310. doi: 10.1038/s41598-019-41655-7. PMCID: PMC6441056.
143. Clark R A, Kupper T S. Misbehaving macrophages in the pathogenesis of psoriasis. J Clin Invest. 2006; 116(8):2084-7. doi: 10.1172/JCI29441. PMCID: PMC1523394.
144. Kasraie S, Werfel T. Role of macrophages in the pathogenesis of atopic dermatitis. Mediators Inflamm. 2013; 2013:942375. doi: 10.1155/2013/942375. PMCID: PMC3603294.
145. Suga H, Sugaya M, Miyagaki T, Ohmatsu H, Kawaguchi M, Takahashi N, Fujita H, Asano Y, Tada Y, Kadono T, Sato S. Skin barrier dysfunction and low antimicrobial peptide expression in cutaneous T-cell lymphoma. Clinical cancer research: an official journal of the American Association for Cancer Research. 2014; 20(16):4339-48. doi: 10.1158/1078-0432.CCR-14-0077.
146. Brown S J, Asai Y, Cordell H J, Campbell L E, Zhao Y, Liao H, Northstone K, Henderson J, Alizadehfar R, Ben-Shoshan M, Morgan K, Roberts G, Masthoff U, Pasmans S G, van den Akker P C, Wijmenga C, Hourihane J O, Palmer C N, Lack G, Clarke A, Hull P R, Irvine A D, McLean W H. Loss-of-function variants in the filaggrin gene are a significant risk factor for peanut allergy. J Allergy Clin Immunol. 2011; 127(3):661-7. doi: 10.1016/j.jaci.2011.01.031. PMCID: 3081065.
147. McAleer M A, Irvine A D. The multifunctional role of filaggrin in allergic skin disease. J Allergy Clin Immunol. 2013; 131(2):280-91. doi: 10.1016/j.jaci.2012.12.668.
148. Irvine A D, McLean W H, Leung D Y. Filaggrin mutations associated with skin and allergic diseases. N Engl J Med. 2011; 365(14):1315-27. doi: 10.1056/NEJMra1011040.
149. Harrop J A, McDonnell P C, Brigham-Burke M, Lyn S D, Minton J, Tan K B, Dede K, Spampanato J, Silverman C, Hensley P, DiPrinzio R, Emery J G, Deen K, Eichman C, Chabot-Fletcher M, Truneh A, Young P R. Herpesvirus entry mediator ligand (HVEM-L), a novel ligand for HVEM/TR2, stimulates proliferation of T cells and inhibits HT29 cell growth. J Biol Chem. 1998; 273(42):27548-56.
150. Morel Y, Schiano De Colella J M, Harrop J, Deen K C, Holmes S D, Wattam T A, Khandekar S S, Truneh A, Sweet R W, Gastaut J A, Olive D, Costello R T. Reciprocal expression of the TNF family receptor herpes virus entry mediator and its ligand LIGHT on activated T cells: LIGHT downregulates its own receptor. J Immunol. 2000; 165(8):4397-404.
151. Tamada K, Shimozaki K, Chapoval A I, Zhai Y, Su J, Chen S F, Hsieh S L, Nagata S, Ni J, Chen L. LIGHT, a TNF-like molecule, costimulates T cell proliferation and is required for dendritic cell-mediated allogeneic T cell response. J Immunol. 2000; 164(8):4105-10.
152. Migone T S, Zhang J, Luo X, Zhuang L, Chen C, Hu B, Hong J S, Perry J W, Chen S F, Zhou J X, Cho Y H, Ullrich S, Kanakaraj P, Carrell J, Boyd E, Olsen H S, Hu G, Pukac L, Liu D, Ni J, Kim S, Gentz R, Feng P, Moore P A, Ruben S M, Wei P. TL1A is a TNF-like ligand for DR3 and TR6/DcR3 and functions as a T cell costimulator. Immunity. 2002; 16(3):479-92.
153. Papadakis K A, Prehn J L, Landers C, Han Q, Luo X, Cha S C, Wei P, Targan S R. TL1A synergizes with IL-12 and IL-18 to enhance IFN-gamma production in human T cells and N K cells. J Immunol. 2004; 172(11): 7002-7.
154. Meylan F, Davidson T S, Kahle E, Kinder M, Acharya K, Jankovic D, Bundoc V, Hodges M, Shevach E M, Keane-Myers A, Wang E C, Siegel R M. The TNF-family receptor DR3 is essential for diverse T cell-mediated inflammatory diseases. Immunity. 2008; 29(1):79-89. doi: S1074-7613(08)00269-0 [pii]10.1016/j.immuni.2008.04.021.
155. McLaren J E, Calder C J, McSharry B P, Sexton K, Salter R C, Singh N N, Wilkinson G W, Wang E C, Ramji D P. The TNF-Like Protein 1A-Death Receptor 3 Pathway Promotes Macrophage Foam Cell Formation In Vitro. J Immunol. 2010; 184(10):5827-34. doi: jimmunol.0903782 [pii]10.4049/jimmunol.0903782.
156. Sibilano R, Gaudenzio N, DeGorter M K, Reber L L, Hernandez J D, Starkl P M, Zurek O W, Tsai M, Zahner S, Montgomery S B, Roers A, Kronenberg M, Yu M, Galli S J. A TNFRSF14-FcvarepsilonRI-mast cell pathway contributes to development of multiple features of asthma pathology in mice. Nat Commun. 2016; 7:13696. doi: 10.1038/ncomms13696. PMCID: PMC5171877.
157. Scholten J, Hartmann K, Gerbaulet A, Krieg T, Muller W, Testa G, Roers A. Mast cell-specific Cre/loxP-mediated recombination in vivo. Transgenic Res. 2008; 17(2):307-15. doi: 10.1007/s11248-007-9153-4. PMCID: PMC2268725.
158. Zhang M, Perrin L, Pardo P. A Randomized Phase 1 Study to Assess the Safety and Pharmacokinetics of the Subcutaneously Injected Anti-LIGHT Antibody, SAR252067. Clin Pharmacol Drug Dev. 2017; 6(3):292-301. doi: 10.1002/cpdd.295.
159. Banfield C, Rudin D, Bhattacharya I, Goteti K, Li G, Hassan-Zahraee M, Brown L S, Hung K E, Pawlak S, Lepsy C. First-in-human, randomized dose-escalation study of the safety, tolerability, pharmacokinetics, pharmacodynamics and immunogenicity of PF-06480605 in healthy subjects. Br J Clin Pharmacol. 2020; 86(4):812-24. doi: 10.1111/bcp.14187. PMCID: PMC7098865.
160. Randall L M, Amante F H, Zhou Y, Stanley A C, Haque A, Rivera F, Pfeffer K, Scheu S, Hill G R, Tamada K, Engwerda C R. Cutting edge: selective blockade of LIGHT-lymphotoxin beta receptor signaling protects mice from experimental cerebral malaria caused by Plasmodium berghei ANKA. J Immunol. 2008; 181(11):7458-62. doi: 181/11/7458 [pii].
161. Xu Y, Flies A S, Flies D B, Zhu G, Anand S, Flies S J, Xu H, Anders R A, Hancock W W, Chen L, Tamada K. Selective targeting of the LIGHT-HVEM costimulatory system for the treatment of graft-versus-host disease. Blood. 2007; 109(9):4097-104.
162. Nakajima K, Sano S. Mouse models of psoriasis and their relevance. J Dermatol. 2018; 45(3):252-63. doi: 10.1111/1346-8138.14112.
163. Chuang S Y, Lin C H, Sung C T, Fang J Y. Murine models of psoriasis and their usefulness for drug discovery. Expert Opin Drug Discov. 2018; 13(6):551-62. doi: 10.1080/17460441.2018.1463214.
164. Kim D, Kobayashi T, Nagao K. Research Techniques Made Simple: Mouse Models of Atopic Dermatitis. J Invest Dermatol. 2019; 139(5):984-90 e1. doi: 10.1016/j.jid.2019.02.014. PMCID: PMC6555635.
165. Jin H, He R, Oyoshi M, Geha R S. Animal models of atopic dermatitis. J Invest Dermatol. 2009; 129(1):31-40. doi: 10.1038/jid.2008.106. PMCID: PMC2886143.

### REFERENCES FOR EXAMPLE 2

1. Davies D E, Wicks J, Powell R M, Puddicombe S M, Holgate S T. Airway remodeling in asthma: new insights. J Allergy Clin Immunol. 2003; 111(2):215-25; quiz 26. Epub 2003/02/18. doi: S0091674902913904 [pii]. PubMed PMID: 12589337.
2. Pascual R M, Peters S P. Airway remodeling contributes to the progressive loss of lung function in asthma: an overview. J Allergy Clin Immunol. 2005; 116(3):477-86; quiz 87. Epub 2005/09/15. doi: S0091-6749(05)01648-9 [pii]10.1016/j.jaci.2005.07.011. PubMed PMID: 16159612.
3. Hassan M, Jo T, Risse P A, Tolloczko B, Lemiere C, Olivenstein R, Hamid Q, Martin J G. Airway smooth muscle remodeling is a dynamic process in severe long-standing asthma. J Allergy Clin Immunol. 2010; 125(5):1037-45 e3. Epub 2010/05/11. doi: 10.1016/j.jaci.2010.02.031. PubMed PMID: 20451038.
4. Hirota N, Martin J G. Mechanisms of airway remodeling. Chest. 2013; 144(3):1026-32. Epub 2013/09/07. doi: 10.1378/chest.12-3073. PubMed PMID: 24008953.
5. Saglani S, Lloyd C M. Novel concepts in airway inflammation and remodelling in asthma. Eur Respir J. 2015; 46(6):1796-804. Epub 2015/11/07. doi: 10.1183/13993003.01196-2014. PubMed PMID: 26541520.
6. Guida G, Riccio A M. Immune induction of airway remodeling. Semin Immunol. 2019; 46:101346. Epub 2019/11/18. doi: 10.1016/j.smim.2019.101346. PubMed PMID: 31734128.
7. Jendzjowsky N G, Kelly M M. The Role of Airway Myofibroblasts in Asthma. Chest. 2019; 156(6):1254-67. Epub 2019/09/01. doi: 10.1016/j.chest.2019.08.1917. PubMed PMID: 31472157.
8. Flood-Page P, Menzies-Gow A, Phipps S, Ying S, Wangoo A, Ludwig M S, Barnes N, Robinson D, Kay A B. Anti-IL-5 treatment reduces deposition of ECM proteins in the bronchial subepithelial basement membrane of mild atopic asthmatics. J Clin Invest. 2003; 112(7):1029-36. Epub 2003/10/03. doi: 10.1172/JCI17974. PubMed PMID: 14523040; PMCID: PMC198522.
9. Riccio A M, Dal Negro R W, Micheletto C, De Ferrari L, Folli C, Chiappori A, Canonica G W. Omalizumab modulates bronchial reticular basement membrane thickness and eosinophil infiltration in severe persistent allergic asthma patients. Int J Immunopathol Pharmacol. 2012; 25(2):475-84. Epub 2012/06/16. doi: 10.1177/039463201202500217. PubMed PMID: 22697079.
10. Panettieri R A, Jr. Airway smooth muscle: an immunomodulatory cell. J Allergy Clin Immunol. 2002; 110(6 Suppl):S269-74. Epub 2002/12/05. doi: 10.1067/mai.2002.129429. PubMed PMID: 12464935.
11. Wenzel S E, Balzar S. Myofibroblast or smooth muscle: do in vitro systems adequately replicate tissue smooth muscle? Am J Respir Crit Care Med. 2006; 174(4):364-5. Epub 2006/08/09. doi: 10.1164/rccm.200606-755E D. PubMed PMID: 16894016.
12. Tliba O, Panettieri R A, Jr. Noncontractile functions of airway smooth muscle cells in asthma. Annu Rev Physiol. 2009; 71:509-35. Epub 2008/10/15. doi: 10.1146/annurev.physiol.010908.163227. PubMed PMID: 18851708.
13. Singh S R, Billington C K, Sayers I, Hall I P. Clonally expanded human airway smooth muscle cells exhibit morphological and functional heterogeneity. Respir Res. 2014; 15:57. Epub 2014/06/03. doi: 10.1186/1465-9921-15-57. PubMed PMID: 24886333; PMCID: PMC4014754.
14. Liu M, Gomez D. Smooth Muscle Cell Phenotypic Diversity. Arterioscler Thromb Vasc Biol. 2019; 39(9): 1715-23. Epub 2019/07/26. doi: 10.1161/ATVBAHA.119.312131. PubMed PMID: 31340668; PMCID: PMC6986347.
15. Chambers R C, Leoni P, Kaminski N, Laurent G J, Heller R A. Global expression profiling of fibroblast responses to transforming growth factor-beta1 reveals the induction of inhibitor of differentiation-1 and provides evidence of smooth muscle cell phenotypic switching. Am J Pathol. 2003; 162(2):533-46. Epub 2003/01/28. doi: 10.1016/s0002-9440(10)63847-3. PubMed PMID: 12547711; PMCID: PMC1851161.
16. Tang D D, Gerlach B D. The roles and regulation of the actin cytoskeleton, intermediate filaments and microtubules in smooth muscle cell migration. Respir Res. 2017; 18(1):54. Epub 2017/04/10. doi: 10.1186/s12931-017-0544-7. PubMed PMID: 28390425; PMCID: PMC5385055.
17. Kuo C, Lim S, King N J, Johnston S L, Burgess J K, Black J L, Oliver B G. Rhinovirus infection induces extracellular matrix protein deposition in asthmatic and nonasthmatic airway smooth muscle cells. Am J Physiol Lung Cell Mol Physiol. 2011; 300(6):L951-7. Epub 2011/04/05. doi: 10.1152/ajplung.00411.2010. PubMed PMID: 21460120.
18. Chen G, Grotendorst G, Eichholtz T, Khalil N. G M-CSF increases airway smooth muscle cell connective tissue expression by inducing TGF-beta receptors. Am J Physiol Lung Cell Mol Physiol. 2003; 284(3):L548-56. Epub 2002/12/10. doi: 10.1152/ajplung.00091.2002. PubMed PMID: 12471017.
19. Coutts A, Chen G, Stephens N, Hirst S, Douglas D, Eichholtz T, Khalil N. Release of biologically active TGF-beta from airway smooth muscle cells induces autocrine synthesis of collagen. Am J Physiol Lung Cell Mol Physiol. 2001; 280(5):L999-1008. Epub 2001/04/06. doi: 10.1152/ajplung.2001.280.5.L999. PubMed PMID: 11290525.
20. Xia Y C, Redhu N S, Moir L M, Koziol-White C, Ammit A J, Al-Alwan L, Camoretti-Mercado B, Clifford R L. Pro-inflammatory and immunomodulatory functions of airway smooth muscle: emerging concepts. Pulm Pharmacol Ther. 2013; 26(1):64-74. Epub 2012/05/29. doi: 10.1016/j.pupt.2012.05.006. PubMed PMID: 22634303.
21. Kinchen J, Chen H I-I, Parikh K, Antanaviciute A, Jagielowicz M, Fawkner-Corbett D, Ashley N, Cubitt L, Mellado-Gomez E, Attar M, Sharma E, Wills Q, Bowden R, Richter F C, Ahern D, Puri K D, Henault J, Gervais F, Koohy H, Simmons A. Structural Remodeling of the Human Colonic Mesenchyme in Inflammatory Bowel Disease. Cell. 2018; 175(2):372-86 e17. Epub 2018/10/03. doi: 10.1016/j.ce11.2018.08.067. PubMed PMID: 30270042; PMCID: PMC6176871.
22. Stephenson W, Donlin L T, Butler A, Rozo C, Bracken B, Rashidfarrokhi A, Goodman S M, Ivashkiv L B, Bykerk V P, Orange D E, Darnell R B, Swerdlow H P, Satija R. Single-cell RNA-seq of rheumatoid arthritis synovial tissue using low-cost microfluidic instrumentation. Nat Commun. 2018; 9(1):791. Epub 2018/02/25. doi: 10.1038/s41467-017-02659-x. PubMed PMID: 29476078; PMCID: PMC5824814.
23. Zhang F, Wei K, Slowikowski K, Fonseka C Y, Rao D A, Kelly S, Goodman S M, Tabechian D, Hughes L B, Salomon-Escoto K, Watts G F M, Jonsson A H, Rangel-Moreno J, Meednu N, Rozo C, Apruzzese W, Eisenhaure T M, Lieb D J, Boyle D L, Mandelin A M, 2nd, Accelerating Medicines Partnership Rheumatoid A, Systemic Lupus Erythematosus C, Boyce B F, DiCarlo E, Gravallese E M, Gregersen P K, Moreland L, Firestein G S, Hacohen N, Nusbaum C, Lederer J A, Perlman H, Pitzalis C, Filer A, Holers V M, Bykerk V P, Donlin L T, Anolik J H, Brenner M B, Raychaudhuri S. Defining inflammatory cell states in rheumatoid arthritis joint synovial tissues by integrating single-cell transcriptomics and mass cytometry. Nat Immunol. 2019; 20(7):928-42. Epub 2019/05/08. doi: 10.1038/s41590-019-0378-1. PubMed PMID: 31061532; PMCID: PMC6602051.
24. Guerrero-Juarez C F, Dedhia P H, Jin S, Ruiz-Vega R, Ma D, Liu Y, Yamaga K, Shestova O, Gay D L, Yang Z, Kessenbrock K, Nie Q, Pear W S, Cotsarelis G, Plikus M V. Single-cell analysis reveals fibroblast heterogeneity and myeloid-derived adipocyte progenitors in murine skin wounds. Nat Commun. 2019; 10(1):650. Epub 2019/02/10. doi: 10.1038/s41467-018-08247-x. PubMed PMID: 30737373; PMCID: PMC6368572.
25. He H, Suryawanshi H, Morozov P, Gay-Mimbrera J, Del Duca E, Kim H J, Kameyama N, Estrada Y, Der E, Krueger J G, Ruano J, Tuschl T, Guttman-Yassky E. Single-cell transcriptome analysis of human skin identifies novel fibroblast subpopulation and enrichment of immune subsets in atopic dermatitis. J Allergy Clin Immunol. 2020. Epub 2020/02/10. doi: 10.1016/j.jaci.2020.01.042. PubMed PMID: 32035984.
26. Kotaru C, Schoonover K J, Trudeau J B, Huynh M L, Zhou X, Hu H, Wenzel S E. Regional fibroblast heterogeneity in the lung: implications for remodeling. Am J Respir Crit Care Med. 2006; 173(11):1208-15. doi: 10.1164/rccm.200508-12180C. PubMed PMID: 16543551; PMCID: PMC2662967.
27. Larsen K, Malmstrom J, Wildt M, Dahlqvist C, Hansson L, Marko-Varga G, Bjermer L, Scheja A, Westergren-Thorsson G. Functional and phenotypical comparison of myofibroblasts derived from biopsies and bronchoalveolar lavage in mild asthma and scleroderma. Respir Res. 2006; 7:11. doi: 10.1186/1465-9921-7-11. PubMed PMID: 16430780; PMCID: PMC1386661.
28. Zhou X, Wu W, Hu H, Milosevic J, Konishi K, Kaminski N, Wenzel S E. Genomic differences distinguish the myofibroblast phenotype of distal lung fibroblasts from airway fibroblasts. Am J Respir Cell Mol Biol. 2011; 45(6):1256-62. Epub 2011/07/16. doi: 10.1165/rcmb.2011-00650C. PubMed PMID: 21757679; PMCID: PMC3262668.
29. Valenzi E, Bulik M, Tabib T, Morse C, Sembrat J, Trejo Bittar H, Rojas M, Lafyatis R. Single-cell analysis reveals fibroblast heterogeneity and myofibroblasts in systemic sclerosis-associated interstitial lung disease. Ann Rheum Dis. 2019; 78(10):1379-87. Epub 2019/08/14. doi: 10.1136/annrheumdis-2018-214865. PubMed PMID: 31405848.
30. Tsukui T, Sun K H, Wetter J B, Wilson-Kanamori J R, Hazelwood L A, Henderson N C, Adams T S, Schupp J C, Poli S D, Rosas T O, Kaminski N, Matthay M A, Wolters P J, Sheppard D. Collagen-producing lung cell atlas identifies multiple subsets with distinct localization and relevance to fibrosis. Nat Commun. 2020; 11(1):1920. Epub 2020/04/23. doi: 10.1038/s41467-020-15647-5. PubMed PMID: 32317643; PMCID: PMC7174390.
31. Xie T, Wang Y, Deng N, Huang G, Taghavifar F, Geng Y, Liu N, Kulur V, Yao C, Chen P, Liu Z, Stripp B, Tang J, Liang J, Noble P W, Jiang D. Single-Cell Deconvolution of Fibroblast Heterogeneity in Mouse Pulmonary Fibrosis. Cell Rep. 2018; 22(13):3625-40. Epub 2018/03/29. doi: 10.1016/j.celrep.2018.03.010. PubMed PMID: 29590628; PMCID: PMC5908225.
32. Peyser R, MacDonnell S, Gao Y, Cheng L, Kim Y, Kaplan T, Ruan Q, Wei Y, Ni M, Adler C, Zhang W, Devalaraja-Narashimha K, Grindley J, Halasz G, Morton L. Defining the Activated Fibroblast Population in Lung Fibrosis Using Single-Cell Sequencing. Am J Respir Cell Mol Biol. 2019; 61(1):74-85. Epub 2019/03/09. doi: 10.1165/rcmb.2018-03130C. PubMed PMID: 30848683.
33. Dobnikar L, Taylor A L, Chappell J, Oldach P, Harman J L, Oerton E, Dzierzak E, Bennett M R, Spivakov M, Jorgensen H F. Disease-relevant transcriptional signatures identified in individual smooth muscle cells from healthy mouse vessels. Nat Commun. 2018; 9(1):4567. Epub 2018/11/06. doi: 10.1038/s41467-018-06891-x. PubMed PMID: 30385745; PMCID: PMC6212435.
34. Danopoulos S, Bhattacharya S, Mariani T J, Al Alam D. Transcriptional characterisation of human lung cells identifies novel mesenchymal lineage markers. Eur Respir J. 2020; 55(1). Epub 2019/10/18. doi: 10.1183/13993003.00746-2019. PubMed PMID: 31619469.
35. Espinosa K, Bosse Y, Stankova J, Rola-Pleszczynski M. CysLT1 receptor upregulation by TGF-beta and IL-13 is associated with bronchial smooth muscle cell proliferation in response to LTD4. J Allergy Clin Immunol. 2003; 111(5):1032-40. Epub 2003/05/14. doi: S009167490301073X [pii]. PubMed PMID: 12743568.
36. Finotto S, Hausding M, Doganci A, Maxeiner J H, Lehr H A, Luft C, Galle P R, Glimcher L H. Asthmatic changes in mice lacking T-bet are mediated by IL-13. Int Immunol. 2005; 17(8):993-1007. Epub 2005/07/08. doi: dxh281 [pii]10.1093/intimm/dxh281. PubMed PMID: 16000330.
37. McMillan S J, Xanthou G, Lloyd C M. Manipulation of allergen-induced airway remodeling by treatment with anti-TGF-beta antibody: effect on the Smad signaling pathway. J Immunol. 2005; 174(9):5774-80. PubMed PMID: 15843580.
38. Doherty T, Broide D. Cytokines and growth factors in airway remodeling in asthma. Curr Opin Immunol. 2007; 19(6):676-80. Epub 2007/08/28. doi: S0952-7915(07)00131-8 [pii]10.1016/j.coi.2007.07.017. PubMed PMID: 17720466.
39. Fulkerson P C, Fischetti C A, Rothenberg M E. Eosinophils and CCR3 regulate interleukin-13 transgene-induced pulmonary remodeling. Am J Pathol. 2006; 169(6):2117-26. Epub 2006/12/07. doi: 169/6/2117 [pii]. PubMed PMID: 17148674; PMCID: 1762480.
40. Boxall C, Holgate S T, Davies D E. The contribution of transforming growth factor-beta and epidermal growth factor signalling to airway remodelling in chronic asthma. Eur Respir J. 2006; 27(1):208-29. Epub 2006/01/03. doi: 27/1/208 [pii]10.1183/09031936.06.00130004. PubMed PMID: 16387953.
41. Lee C G, Cho S J, Kang M J, Chapoval S P, Lee P J, Noble P W, Yehualaeshet T, Lu B, Flavell R A, Milbrandt J, Homer R J, Elias J A. Early growth response gene 1-mediated apoptosis is essential for transforming growth factor beta1-induced pulmonary fibrosis. J Exp Med. 2004; 200(3):377-89. Epub 2004/08/04. doi: 10.1084/jem.20040104jem.20040104 [pii]. PubMed PMID: 15289506; PMCID: 2211975.
42. Broide D H. Immunologic and inflammatory mechanisms that drive asthma progression to remodeling. J Allergy Clin Immunol. 2008; 121(3):560-70. Epub 2008/03/11. doi: S0091-6749(08)00231-5 [pii]10.1016/j.jaci.2008.01.031. PubMed PMID: 18328887; PMCID: 2386668.
43. Croft M. Co-stimulatory members of the TNFR family: keys to effective T-cell immunity? Nature reviews Immunology. 2003; 3(8):609-20. Epub 2003/09/17. doi: 10.1038/nri1148. PubMed PMID: 12974476.
44. Croft M. The role of TNF superfamily members in T-cell function and diseases. Nat Rev Immunol. 2009; 9(4):271-85. Epub 2009/03/26. doi: nri2526 [pii]10.1038/nri2526. PubMed PMID: 19319144.
45. Doherty T A, Croft M. Therapeutic potential of targeting TNF/TNFR family members in asthma. Immunotherapy. 2011; 3(8):919-21. Epub 2011/08/17. doi: 10.2217/imt.11.88. PubMed PMID: 21843077.
46. Croft M, Duan W, Choi H, Eun S Y, Madireddi S, Mehta A. TNF superfamily in inflammatory disease: translating basic insights. Trends Immunol. 2012; 33(3):144-52. Epub 2011/12/16. doi: 10.1016/j.it.2011.10.004. PubMed PMID: 22169337; PMCID: 3299395.
47. Croft M, Benedict C A, Ware C F. Clinical targeting of the TNF and TNFR superfamilies. Nat Rev Drug Discov. 2013; 12(2):147-68. Epub 2013/01/22. doi: 10.1038/nrd3930. PubMed PMID: 23334208; PMCID: 3625401.
48. Doherty T A, Soroosh P, Khorram N, Fukuyama S, Rosenthal P, Cho J Y, Norris P S, Choi H, Scheu S, Pfeffer K, Zuraw B L, Ware C F, Broide D H, Croft M. The tumor necrosis factor family member LIGHT is a target for asthmatic airway remodeling. Nature Medicine. 2011; 17(5):596-603. Epub 2011/04/19. doi: nm.2356 [pii]10.1038/nm.2356. PubMed PMID: 21499267; PMCID: 3097134.
49. Soroosh P, Doherty T A, So T, Mehta A K, Khorram N, Norris P S, Scheu S, Pfeffer K, Ware C, Croft M. Herpesvirus entry mediator (TNFRSF14) regulates the persistence of T helper memory cell populations. Journal of Experimental Medicine. 2011; 208(4):797-809. Epub 2011/03/16. doi: 10.1084/jem.20101562. PubMed PMID: 21402741; PMCID: 3135347.
50. Sibilano R, Gaudenzio N, DeGorter M K, Reber L L, Hernandez J D, Starkl P M, Zurek O W, Tsai M, Zahner S, Montgomery S B, Roers A, Kronenberg M, Yu M, Galli S J. A TNFRSF14-FcvarepsilonRl-mast cell pathway contributes to development of multiple features of asthma pathology in mice. Nat Commun. 2016; 7:13696. Epub 2016/12/17. doi: 10.1038/ncomms13696. PubMed PMID: 27982078; PMCID: PMC5171877.
51. Meylan F, Davidson T S, Kahle E, Kinder M, Acharya K, Jankovic D, Bundoc V, Hodges M, Shevach E M, Keane-Myers A, Wang E C, Siegel R M. The TNF-family receptor DR3 is essential for diverse T cell-mediated inflammatory diseases. Immunity. 2008; 29(1):79-89. Epub 2008/06/24. doi: S1074-7613(08)00269-0 [pii]10.1016/j.immuni.2008.04.021. PubMed PMID: 18571443.
52. Meylan F, Hawley E T, Barron L, Barlow J L, Penumetcha P, Pelletier M, Sciume G, Richard A C, Hayes E T, Gomez-Rodriguez J, Chen X, Paul W E, Wynn T A, McKenzie A N, Siegel R M. The TNF-family cytokine TL1A promotes allergic immunopathology through group 2 innate lymphoid cells. Mucosal Immunol. 2014; 7(4):958-68. doi: 10.1038/mi.2013.114. PubMed PMID: 24368564; PMCID: 4165592.
53. da Silva Antunes R, Madge L, Soroosh P, Tocker J, Croft M. The TNF Family Molecules LIGHT and Lymphotoxin alphabeta Induce a Distinct Steroid-Resistant Inflammatory Phenotype in Human Lung Epithelial Cells. J Immunol. 2015; 195(5):2429-41. doi: 10.4049/jimmunol.1500356. PubMed PMID: 26209626; PMCID: 4546856.
54. Herro R, Da Silva Antunes R, Aguilera A R, Tamada K, Croft M. Tumor necrosis factor superfamily 14 (LIGHT) controls thymic stromal lymphopoietin to drive pulmonary fibrosis. J Allergy Clin Immunol. 2015; 136(3):757-68. doi: 10.1016/j.jaci.2014.12.1936. PubMed PMID: 25680454; PMCID: 4532661.
55. Herro R, Antunes Rda S, Aguilera A R, Tamada K, Croft M. The Tumor Necrosis Factor Superfamily Molecule LIGHT Promotes Keratinocyte Activity and Skin Fibrosis. J Invest Dermatol. 2015; 135(8):2109-18. doi: 10.1038/jid.2015.110. PubMed PMID: 25789702; PMCID: 4504809.
56. Mehta A K, Duan W, Doerner A M, Traves S L, Broide D H, Proud D, Zuraw B L, Croft M. Rhinovirus infection interferes with induction of tolerance to aeroantigens through OX40 ligand, thymic stromal lymphopoietin, and IL-33. J Allergy Clin Immunol. 2016; 137(1):278-88 e6. Epub 2015/06/24. doi: 10.1016/j.jaci.2015.05.007. PubMed PMID: 26100084; PMCID: PMC4684822.
57. Miller M, Beppu A, Rosenthal P, Pham A, Das S, Karta M, Song D J, Vuong C, Doherty T, Croft M, Zuraw B, Zhang X, Gao X, Aceves S, Chouiali F, Hamid Q, Broide D H. Fstll Promotes Asthmatic Airway Remodeling by Inducing Oncostatin M. J Immunol. 2015; 195(8):3546-56. doi: 10.4049/jimmunol.1501105. PubMed PMID: 26355153.
58. Mehta A K, Doherty T, Broide D, Croft M. Tumor necrosis factor family member LIGHT acts with IL-1beta and TGF-beta to promote airway remodeling during rhinovirus infection. Allergy. 2018; 73(7):1415-24. Epub 2018/01/10. doi: 10.1111/a11.13390. PubMed PMID: 29315623; PMCID: PMC6019192.
59. Herro R, Shui J W, Zahner S, Sidler D, Kawakami Y, Kawakami T, Tamada K, Kronenberg M, Croft M. LIGHT-HVEM signaling in keratinocytes controls development of dermatitis. J Exp Med. 2018; 215(2):415-22. Epub 2018/01/18. doi: 10.1084/jem.20170536. PubMed PMID: 29339444; PMCID: PMC5789407.
60. da Silva Antunes R, Mehta A K, Madge L, Tocker J, Croft M. TNFSF14 (LIGHT) Exhibits Inflammatory Activities in Lung Fibroblasts Complementary to IL-13 and TGF-beta. Front Immunol. 2018; 9:576. Epub 2018/04/05. doi: 10.3389/fimmu.2018.00576. PubMed PMID: 29616048; PMCID: PMC5868327.
61. Croft M, Siegel R M. Beyond TNF: TNF superfamily cytokines as targets for the treatment of rheumatic diseases. Nat Rev Rheumatol. 2017; 13(4):217-33. doi: 10.1038/nrrheum.2017.22. PubMed PMID: 28275260.
62. Herro R, Croft M. The control of tissue fibrosis by the inflammatory molecule LIGHT (TNF Superfamily member 14). Pharmacol Res. 2016; 104:151-5. Epub 2016/01/10. doi: 10.1016/j.phrs.2015.12.018. PubMed PMID: 26748035; PMCID: PMC4712451.
63. Miller M, Esnault S, Kurten R C, Kelly E A, Beppu A, Das S, Rosenthal P, Ramsdell J, Croft M, Zuraw B, Jarjour N, Hamid Q, Broide D H. Segmental allergen challenge increases levels of airway follistatin-like 1 in patients with asthma. J Allergy Clin Immunol. 2016; 138(2):596-9 e4. doi: 10.1016/j.jaci.2016.01.019. PubMed PMID: 27001159; PMCID: PMC4975994.
64. Lund S J, Portillo A, Cavagnero K, Baum R E, Naji L H, Badrani J H, Mehta A, Croft M, Broide D H, Doherty T A. Leukotriene C4 Potentiates IL-33-Induced Group 2 Innate Lymphoid Cell Activation and Lung Inflammation. J Immunol. 2017; 199(3):1096-104. Epub 2017/07/02. doi: 10.4049/jimmunol.1601569. PubMed PMID: 28667163; PMCID: PMC5531601.
65. Song D J, Miller M, Beppu A, Rosenthal P, Das S, Karta M, Vuong C, Mehta A K, Croft M, Broide D H. Rhinovirus Infection of ORMDL3 Transgenic Mice Is Associated with Reduced Rhinovirus Viral Load and Airway Inflammation. J Immunol. 2017; 199(7):2215-24. Epub 2017/08/23. doi: 10.4049/jimmunol.1601412. PubMed PMID: 28827284; PMCID: PMC5605463.
66. Mehta A K, Gracias D T, Croft M. TNF activity and T cells. Cytokine. 2018; 101:14-8. Epub 2016/08/18. doi: 10.1016/j.cyto.2016.08.003. PubMed PMID: 27531077; PMCID: PMC5305780.
67. Cavagnero K J, Badrani J H, Naji L H, Amadeo M B, Shah V S, Gasparian S, Pham A, Wang A W, Seumois G, Croft M, Broide D H, Doherty T A. Unconventional ST2- and CD127-negative lung ILC2 populations are induced by the fungal allergen Alternaria alternata. J Allergy Clin Immunol. 2019; 144(5):1432-5 e9. Epub 2019/08/02. doi: 10.1016/j.jaci.2019.07.018. PubMed PMID: 31369800; PMCID: PMC6938690.
68. Mehta A K, Croft M. Rhinovirus infection promotes eosinophilic airway inflammation after prior exposure to house dust mite allergen. ImmunoHorizons. 2020; in press.
69. Herro R, Mills D, Mehta A K, Nguyen X-X, Feghali-Bostwick C, Miller M, Broide D, Soloff R, Croft M. TL1A promotes lung tissue fibrosis and airway remodeling. J Immunol. 2020; in press.
70. Ray A, Oriss T B, Wenzel S E. Emerging molecular phenotypes of asthma. Am J Physiol Lung Cell Mol Physiol. 2015; 308(2):L130-40. doi: 10.1152/ajplung.00070.2014. PubMed PMID: 25326577; PMCID: 4338947.
71. Raundhal M, Morse C, Khare A, Oriss T B, Milosevic J, Trudeau J, Huff R, Pilewski J, Holguin F, Kolls J, Wenzel S, Ray P, Ray A. High IFN-gamma and low SLPI mark severe asthma in mice and humans. J Clin Invest. 2015; 125(8):3037-50. doi: 10.1172/JCI80911. PubMed PMID: 26121748; PMCID: 4563754.
72. Chambers E S, Nanzer A M, Pfeffer P E, Richards D F, Timms P M, Martineau A R, Griffiths C J, Corrigan C J, Hawrylowicz C M. Distinct endotypes of steroid-resistant asthma characterized by I L-17A(high) and IFN-gamma(high) immunophenotypes: Potential benefits of calcitriol. J Allergy Clin Immunol. 2015; 136(3):628-37 e4. doi: 10.1016/j.jaci.2015.01.026. PubMed PMID: 25772594; PMCID: 4559139.
73. Hastie A T, Moore W C, Meyers D A, Vestal P L, Li H, Peters S P, Bleecker E R. Analyses of asthma severity phenotypes and inflammatory proteins in subjects stratified by sputum granulocytes. J Allergy Clin Immunol. 2010; 125(5):1028-36. Epub 2010/04/20. doi: S0091-6749(10)00289-7 [pii]10.1016/j.jaci.2010.02.008. PubMed PMID: 20398920; PMCID: 2878277.
74. Kuo C S, Pavlidis S, Loza M, Baribaud F, Rowe A, Pandis I, Sousa A, Corfield J, Djukanovic R, Lutter R, Sterk P J, Auffray C, Guo Y, Adcock I M, Chung K F, Group UBS. T-helper cell type 2 (Th2) and non-Th2 molecular phenotypes of asthma using sputum transcriptomics in U-BIOPRED. Eur Respir J. 2017; 49(2). Epub 2017/02/10. doi: 10.1183/13993003.02135-2016. PubMed PMID: 28179442.
75. Boussaud V, Soler P, Moreau J, Goodwin R G, Hance A J. Expression of three members of the TNF-R family of receptors (4-1B B, lymphotoxin-beta receptor, and Fas) in human lung. European Respiratory Journal. 1998; 12(4):926-31. Epub 1998/11/17. PubMed PMID: 9817170.
76. Jung H W, La S J, Kim J Y, Heo S K, Wang S, Kim K K, Lee K M, Cho H R, Lee H W, Kwon B, Kim B S, Kwon B S. High levels of soluble herpes virus entry mediator in sera of patients with allergic and autoimmune diseases. Exp Mol Med. 2003; 35(6):501-8. PubMed PMID: 14749527.
77. Kong Q, Li W J, Huang H R, Zhong Y Q, Fang J P. Differential gene expression profiles of peripheral blood mononuclear cells in childhood asthma. The Journal of asthma: official journal of the Association for the Care of Asthma. 2015; 52(4):343-52. doi: 10.3109/02770903.2014.971967. PubMed PMID: 25329679.
78. Esnault S, Kelly E A, Schwantes E A, Liu L Y, DeLain L P, Hauer J A, Bochkov Y A, Denlinger L C, Malter J S, Mathur S K, Jarjour N N. Identification of genes expressed by human airway eosinophils after an in vivo allergen challenge. PloS one. 2013; 8(7):e67560. Epub 2013/07/12. doi: 10.1371/journal.pone.0067560. PubMed PMID: 23844029; PMCID: 3699655.
79. Otterdal K, Andreassen A K, Yndestad A, Oie E, Sandberg W J, Dahl C P, Pedersen T M, Ueland T, Gullestad L, Brosstad F R, Aukrust P, Damas J K. Raised LIGHT levels in pulmonary arterial hypertension: potential role in thrombus formation. Am J Respir Crit Care Med. 2008; 177(2):202-7. Epub 2007/10/27. doi: 200703-5060C [pii]10.1164/rccm.200703-5060C. PubMed PMID: 17962639.
80. Seumois G, Ramirez-Suastegui C, Schmiedel B J, Liang S, Peters B, Sette A, Vijayanand P. Single-cell transcriptomic analysis of allergen-specific T cells in allergy and asthma. Sci Immunol. 2020; 5(48). Epub 2020/06/14. doi: 10.1126/sciimmunol.aba6087. PubMed PMID: 32532832; PMCID: PMC7372639.
81. Manresa M C, Chiang A W T, Kurten R C, Dohil R, Brickner H, Dohil L, Herro R, Akuthota P, Lewis N E, Croft M, Aceves S S. Increased production of LIGHT by T cells in eosinophilic esophagitis promotes differentiation of esophageal fibroblasts toward an inflammatory phenotype. Gastroenterology. 2020; in press.
82. Kotani H, Masuda K, Tamagawa-Mineoka R, Nomiyama T, Soga F, Nin M, Asai J, Kishimoto S, Katoh N. Increased plasma LIGHT levels in patients with atopic dermatitis. Clinical and experimental immunology. 2012; 168(3): 318-24. Epub 2012/04/24. doi: 10.1111/j.1365-2249.2012.04576.x. PubMed PMID: 22519595; PMCID: 3390484.
83. Morimura S, Sugaya M, Kai H, Kato T, Miyagaki T, Ohmatsu H, Kagami S, Asano Y, Mitsui H, Tada Y, Kadono T, Sato S. High levels of LIGHT and low levels of soluble herpesvirus entry mediator in sera of patients with atopic dermatitis. Clin Exp Dermatol. 2012; 37(2):181-2. Epub 2011/06/01. doi: 10.1111/j.1365-2230.2011.04079.x. PubMed PMID: 21623877.
84. Gindzienska-Sieskiewicz E, Distler O, Reszec J, Jordan S, Bielecki P, Sieskiewicz A, Sulik A, Lukasik M, Bielecki M, Kowal K, Kowal-Bielecka O. Increased expression of the TNF superfamily member LIGHT/TNFSF14 and its receptors (HVEM and LTssR) in patients with systemic sclerosis. Rheumatology (Oxford). 2019; 58(3):502-10. Epub 2018/12/07. doi: 10.1093/rheumatology/key348. PubMed PMID: 30508197.
85. Tsoi L C, Rodriguez E, Degenhardt F, Baurecht H, Wehkamp U, Volks N, Szymczak S, Swindell W R, Sarkar M K, Raja K, Shao S, Patrick M, Gao Y, Uppala R, Perez White B E, Getsios S, Harms P W, Maverakis E, Elder J T, Franke A, Gudjonsson J E, Weidinger S. Atopic Dermatitis Is an IL-13-Dominant Disease with Greater Molecular Heterogeneity Compared to Psoriasis. J Invest Dermatol. 2019; 139(7):1480-9. Epub 2019/01/15. doi: 10.1016/j.jid.2018.12.018. PubMed PMID: 30641038; PMCID: PMC6711380.
86. Machida K, Aw M, Salter B M, Ju X, Mukherjee M, Gauvreau G M, O'Byrne P M, Nair P, Sehmi R. Role of TL1A/DR3 Axis in the Activation of ILC2s in Eosinophilic Asthmatics. Am J Respir Crit Care Med. 2020. Epub 2020/06/26. doi: 10.1164/rccm.201909-17220C. PubMed PMID: 32584596.
87. Bamias G, Evangelou K, Vergou T, Tsimaratou K, Kaltsa G, Antoniou C, Kotsinas A, Kim S, Gorgoulis V, Stratigos A J, Sfikakis P P. Upregulation and nuclear localization of TNF-like cytokine 1A (TL1A) and its receptors DR3 and DcR3 in psoriatic skin lesions. Experimental dermatology. 2011; 20(9):725-31. doi: 10.1111/j.1600-0625.2011.01304.x. PubMed PMID: 21672030.
88. Li L, Fu L, Lu Y, Wang W, Liu H, Li F, Chen T. TNF-like ligand 1A is associated with the pathogenesis of psoriasis vulgaris and contributes to IL-17 production in PBMCs. Arch Dermatol Res. 2014; 306(10):927-32. doi: 10.1007/s00403-014-1497-z. PubMed PMID: 25200589.
89. Pedersen A E, Schmidt E G, Sorensen J F, Faber C, Nielsen B S, Holmstrom K, Omland S H, Tougaard P, Skov S, Bang B. Secretion, blood levels and cutaneous expression of TL1A in psoriasis patients. APMIS. 2015; 123(7):547-55. doi: 10.1111/apm.12385. PubMed PMID: 25908025.
90. Li L, Lu Y, Fu L, Zhou P, Zhang L, Wang W, Nie J, Zhang D, Liu Y, Wu B, Zhou Y, Chen T. Expression of death receptor 3 (DR3) on peripheral blood mononuclear cells of patients with psoriasis vulgaris. Postgrad Med J. 2018; 94(1116):551-5. Epub 2018/10/21. doi: 10.1136/postgradmedj-2018-136040. PubMed PMID: 30341229.
91. Xu W, Su L, Qing P, Wang Y, Liang Y, Zhao Y, Zhou Q, Ma F, Liu Y. Elevated levels of TL1A are associated with disease activity in patients with systemic sclerosis. Clin Rheumatol. 2017; 36(6):1317-24. Epub 2017/04/12. doi: 10.1007/s10067-017-3612-y. PubMed PMID: 28397078.
92. Facco M, Cabrelle A, Calabrese F, Teramo A, Cinetto F, Carraro S, Martini V, Calzetti F, Tamassia N, Cassatella M A, Semenzato G, Agostini C. TL1A/DR3 axis involvement in the inflammatory cytokine network during pulmonary sarcoidosis. Clin Mol Allergy. 2015; 13(1):16. doi: 10.1186/s12948-015-0022-z. PubMed PMID: 26240517; PMCID: 4522997.
93. Zhang M, Perrin L, Pardo P. A Randomized Phase 1 Study to Assess the Safety and Pharmacokinetics of the Subcutaneously Injected Anti-LIGHT Antibody, SAR252067. Clin Pharmacol Drug Dev. 2017; 6(3):292-301. Epub 2016/08/23. doi: 10.1002/cpdd.295. PubMed PMID: 27545119.
94. Banfield C, Rudin D, Bhattacharya I, Goteti K, Li G, Hassan-Zahraee M, Brown L S, Hung K E, Pawlak S, Lepsy C. First-in-human, randomized dose-escalation study of the safety, tolerability, pharmacokinetics, pharmacodynamics and immunogenicity of PF-06480605 in healthy subjects. Br J Clin Pharmacol. 2020; 86(4):812-24. Epub 2019/11/24. doi: 10.1111/bcp.14187. PubMed PMID: 31758576; PMCID: PMC7098865.
95. Vieira Braga F A, Kar G, Berg M, Carpaij O A, Polanski K, Simon L M, Brouwer S, Gomes T, Hesse L, Jiang J, Fasouli E S, Efremova M, Vento-Tormo R, Talavera-Lopez C, Jonker M R, Affleck K, Palit S, Strzelecka P M, Firth H V, Mahbubani K T, Cvejic A, Meyer K B, Saeb-Parsy K, Luinge M, Brandsma C A, Timens W, Angelidis I, Strunz M, Koppelman G H, van Oosterhout A J, Schiller H B, Theis F J, van den Berge M, Nawijn M C, Teichmann S A. A cellular census of human lungs identifies novel cell states in health and in asthma. Nat Med. 2019; 25(7):1153-63. Epub 2019/06/19. doi: 10.1038/s41591-019-0468-5. PubMed PMID: 31209336.
96. Evasovic J M, Singer C A. Regulation of IL-17A and implications for TGF-beta1 comodulation of airway smooth muscle remodeling in severe asthma. Am J Physiol Lung Cell Mol Physiol. 2019; 316(5):L843-L68. Epub 2019/02/28. doi: 10.1152/ajplung.00416.2018. PubMed PMID: 30810068; PMCID: PMC6589583.
97. Ye W J, Xu W G, Guo X J, Han F F, Peng J, Li X M, Guan W B, Yu L W, Sun J Y, Cui Z L, Song L, Zhang Y, Wang Y M, Yang T Y, Ge X H, Yao D, Liu S. Differences in airway remodeling and airway inflammation among moderate-severe asthma clinical phenotypes. J Thorac Dis. 2017; 9(9):2904-14. Epub 2017/12/10. doi: 10.21037/jtd.2017.08.01. PubMed PMID: 29221262; PMCID: PMC5708482.
98. Ricciardolo F L M, Sorbello V, Folino A, Gallo F, Massaglia G M, Favata G, Conticello S, Vallese D, Gani F, Malerba M, Folkerts G, Rolla G, Profita M, Mauad T, Di Stefano A, Ciprandi G. Identification of IL-17F/frequent exacerbator endotype in asthma. J Allergy Clin Immunol. 2017; 140(2):395-406. Epub 2016/12/10. doi: 10.1016/j.jaci.2016.10.034. PubMed PMID: 27931975.
99. Doucet C, Brouty-Boye D, Pottin-Clemenceau C, Jasmin C, Canonica G W, Azzarone B. IL-4 and IL-13 specifically increase adhesion molecule and inflammatory cytokine expression in human lung fibroblasts. Int Immunol. 1998; 10(10):1421-33. PubMed PMID: 9796908.
100. Lee J H, Kaminski N, Dolganov G, Grunig G, Koth L, Solomon C, Erle D J, Sheppard D. Interleukin-13 induces dramatically different transcriptional programs in three human airway cell types. Am J Respir Cell Mol Biol. 2001; 25(4):474-85. doi: 10.1165/ajrcmb.25.4.4522. PubMed PMID: 11694453.
101. Henness S, Johnson C K, Ge Q, Armour C L, Hughes J M, Ammit A J. IL-17A augments TNF-alpha-induced IL-6 expression in airway smooth muscle by enhancing mRNA stability. J Allergy Clin Immunol. 2004; 114(4):958-64. Epub 2004/10/14. doi: 10.1016/j.jaci.2004.06.023. PubMed PMID: 15480342.
102. Kudo M, Melton A C, Chen C, Engler M B, Huang K E, Ren X, Wang Y, Bernstein X, Li J T, Atabai K, Huang X, Sheppard D. IL-17A produced by alphabeta T cells drives airway hyper-responsiveness in mice and enhances mouse and human airway smooth muscle contraction. Nat Med. 2012; 18(4):547-54. Epub 2012/03/06. doi: 10.1038/nm.2684. PubMed PMID: 22388091; PMCID: PMC3321096.
103. Bulek K, Chen X, Parron V, Sundaram A, Herjan T, Ouyang S, Liu C, Majors A, Zepp J, Gao J, Dongre A, Bodaszewska-Lubas M, Echard A, Aronica M, Carman J, Garantziotis S, Sheppard D, Li X. IL-17A Recruits Rab35 to IL-17R to Mediate PKCalpha-Dependent Stress Fiber Formation and Airway Smooth Muscle Contractility. J Immunol. 2019; 202(5):1540-8. Epub 2019/01/27. doi: 10.4049/jimmunol.1801025. PubMed PMID: 30683702; PMCID: PMC6379809.
104. Tliba O, Deshpande D, Chen H, Van Besien C, Kalman M, Panettieri R A, Jr., Amrani Y. IL-13 enhances agonist-evoked calcium signals and contractile responses in airway smooth muscle. Br J Pharmacol. 2003; 140(7):1159-62. Epub 2003/11/05. doi: 10.1038/sj.bjp.0705558. PubMed PMID: 14597600; PMCID: PMC1574143.
105. Ohta Y, Hayashi M, Kanemaru T, Abe K, Ito Y, Oike M. Dual modulation of airway smooth muscle contraction by Th2 cytokines via matrix metalloproteinase-1 production. J Immunol. 2008; 180(6):4191-9. Epub 2008/03/07. doi: 10.4049/jimmunol.180.6.4191. PubMed PMID: 18322231.
106. Chang Y, Al-Alwan L, Risse P A, Roussel L, Rousseau S, Halayko A J, Martin J G, Hamid Q, Eidelman D H. TH17 cytokines induce human airway smooth muscle cell migration. J Allergy Clin Immunol. 2011; 127(4):1046-53 e1-2. Epub 2011/02/25. doi: 10.1016/j.jaci.2010.12.1117. PubMed PMID: 21345484.
107. Wenzel S E, Trudeau J B, Barnes S, Zhou X, Cundall M, Westcott J Y, McCord K, Chu H W. TGF-beta and IL-13 synergistically increase eotaxin-1 production in human airway fibroblasts. J Immunol. 2002; 169(8):4613-9. PubMed PMID: 12370400.
108. Zhou X, Trudeau J B, Schoonover K J, Lundin J I, Barnes S M, Cundall M J, Wenzel S E. Interleukin-13 augments transforming growth factor-beta1-induced tissue inhibitor of metalloproteinase-1 expression in primary human airway fibroblasts. Am J Physiol Cell Physiol. 2005; 288(2):C435-42. doi: 10.1152/ajpce11.00035.2004. PubMed PMID: 15456694.
109. Hall S L, Baker T, Lajoie S, Richgels P K, Yang Y, McAlees J W, van Lier A, Wills-Karp M, Sivaprasad U, Acciani T H, LeCras T D, Myers J B, Kovacic M B, Lewkowich I P. IL-17A enhances IL-13 activity by enhancing IL-13-induced signal transducer and activator of transcription 6 activation. J Allergy Clin Immunol. 2016. doi: 10.1016/j.j aci 0.2016.04.037. PubMed PMID: 27417023.
110. Hur G Y, Pham A, Miller M, Weng N, Hu J, Kurten R C, Broide D H. ORMDL3 but not neighboring 17q21 gene LRRC3C is expressed in human lungs and lung cells of asthmatics. Allergy. 2020. Epub 2020/02/23. doi: 10.1111/a11.14243. PubMed PMID: 32086831.
111. Sakota Y, Ozawa Y, Yamashita H, Tanaka H, Inagaki N. Collagen gel contraction assay using human bronchial smooth muscle cells and its application for evaluation of inhibitory effect of formoterol. Biol Pharm Bull. 2014; 37(6):1014-20. PubMed PMID: 24882412.
112. Beppu L Y, Anilkumar A A, Newbury R O, Dohil R, Broide D H, Aceves S S. TGF-beta1-induced phospholamban expression alters esophageal smooth muscle cell contraction in patients with eosinophilic esophagitis. J Allergy Clin Immunol. 2014; 134(5):1100-7 e4. doi: 10.1016/j.jaci.2014.04.004. PubMed PMID: 24835503; PMCID: 4231011.
113. Farghaly H S, Blagbrough I S, Medina-Tato D A, Watson M L. Interleukin 13 increases contractility of murine tracheal smooth muscle by a phosphoinositide 3-kinase p 110delta-dependent mechanism. Mol Pharmacol. 2008; 73(5):1530-7. Epub 2008/02/16. doi: 10.1124/mol.108.045419. PubMed PMID: 18276774.
114. Steinberg M W, Cheung T C, Ware C F. The signaling networks of the herpesvirus entry mediator (TNFRSF14) in immune regulation. Immunol Rev. 2011; 244(1):169-87. Epub 2011/10/25. doi: 10.1111/j.1600-065X.2011.01064.x. PubMed PMID: 22017438; PMCID: 3381650.
115. Ware C F, Sedy J R. TNF Superfamily Networks: bidirectional and interference pathways of the herpesvirus entry mediator (TNFSF14). Curr Opin Immunol. 2011; 23(5):627-31. Epub 2011/09/17. doi: 10.1016/j.coi.2011.08.008. PubMed PMID: 21920726; PMCID: 3222271.
116. Chinnaiyan A M, O'Rourke K, Yu G L, Lyons R H, Garg M, Duan D R, Xing L, Gentz R, Ni J, Dixit V M. Signal transduction by DR3, a death domain-containing receptor related to TNFR-1 and CD95. Science. 1996; 274(5289):990-2. Epub 1996/11/08. PubMed PMID: 8875942.
117. Wen L, Zhuang L, Luo X, Wei P. TL1A-induced NF-kappaB activation and c-IAP2 production prevent DR3-mediated apoptosis in TF-1 cells. J Biol Chem. 2003; 278(40):39251-8. doi: 10.1074/jbc.M305833200. PubMed PMID: 12882979.
118. Pobezinskaya Y L, Choksi S, Morgan M J, Cao X, Liu Z G. The Adaptor Protein TRADD Is Essential for TNF-Like Ligand 1A/Death Receptor 3 Signaling. J Immunol. 2011; 186(9):5212-6. Epub 2011/03/23. doi: jimmunol.1002374 [pii]10.4049/jimmunol.1002374. PubMed PMID: 21421854; PMCID: 3080469.
119. Marsters S A, Ayres T M, Skubatch M, Gray C L, Rothe M, Ashkenazi A. Herpesvirus entry mediator, a member of the tumor necrosis factor receptor (TNFR) family, interacts with members of the TNFR-associated factor family and activates the transcription factors NF-kappaB and AP-1. J Biol Chem. 1997; 272(22):14029-32. PubMed PMID: 9162022.
120. Dejardin E, Droin N M, Delhase M, Haas E, Cao Y, Makris C, Li Z W, Karin M, Ware C F, Green D R. The lymphotoxin-beta receptor induces different patterns of gene expression via two NF-kappaB pathways. Immunity. 2002; 17(4):525-35. PubMed PMID: 12387745.
121. Muller J R, Siebenlist U. Lymphotoxin beta receptor induces sequential activation of distinct NF-kappa B factors via separate signaling pathways. J Biol Chem. 2003; 278(14):12006-12. PubMed PMID: 12556537.
122. Hauer J, Puschner S, Ramakrishnan P, Simon U, Bongers M, Federle C, Engelmann H. TNF receptor (TNFR)-associated factor (TRAF) 3 serves as an inhibitor of TRAF2/5-mediated activation of the noncanonical NF-kappaB pathway by TRAF-binding TNFRs. Proc Natl Acad Sci USA. 2005; 102(8):2874-9. PubMed PMID: 15708970.
123. Shui J W, Larange A, Kim G, Vela J L, Zahner S, Cheroutre H, Kronenberg M. HVEM signalling at mucosal barriers provides host defence against pathogenic bacteria. Nature. 2012; 488(7410):222-5. Epub 2012/07/18. doi: 10.1038/nature11242. PubMed PMID: 22801499; PMCID: 3477500.
124. So T, Croft M. Regulation of PI-3-Kinase and Akt Signaling in T Lymphocytes and Other Cells by TNFR Family Molecules. Frontiers in immunology. 2013; 4:139. Epub 2013/06/14. doi: 10.3389/fimmu.2013.00139. PubMed PMID: 23760533; PMCID: 3675380.
125. Lee S H, Kim E J, Suk K, Kim I S, Lee W H. TL1A induces the expression of TGF-beta-inducible gene h3 (betaig-h3) through PKC, PI3K, and ERK in THP-1 cells. Cell Immunol. 2010; 266(1):61-6. Epub 2010/09/25. doi: 10.1016/j.cellimm.2010.08.013. PubMed PMID: 20863486.
126. McFawn P K, Shen L, Vincent S G, Mak A, Van Eyk J E, Fisher J T. Calcium-independent contraction and sensitization of airway smooth muscle by p21-activated protein kinase. Am J Physiol Lung Cell Mol Physiol. 2003; 284(5):L863-70. Epub 2003/01/07. doi: 10.1152/ajplung.00068.2002. PubMed PMID: 12513968.
127. Fajmut A, Brumen M. MLC-kinase/phosphatase control of Ca2+ signal transduction in airway smooth muscles. J Theor Biol. 2008; 252(3):474-81. Epub 2007/11/17. doi: 10.1016/j.jtbi.2007.10.005. PubMed PMID: 18005997.
128. Zhang W, Du L, Gunst S J. The effects of the small GTPase RhoA on the muscarinic contraction of airway smooth muscle result from its role in regulating actin polymerization. Am J Physiol Cell Physiol. 2010; 299(2):C298-306. Epub 2010/05/07. doi: 10.1152/ajpcell.00118.2010. PubMed PMID: 20445174; PMCID: PMC2928621.
129. Zhang W, Huang Y, Gunst S J. p21-Activated kinase (Pak) regulates airway smooth muscle contraction by regulating paxillin complexes that mediate actin polymerization. J Physiol. 2016; 594(17):4879-900. Epub 2016/04/03. doi: 10.1113/JP272132. PubMed PMID: 27038336; PMCID: PMC5009781.
130. Kume H. RhoA/Rho-kinase as a therapeutic target in asthma. Curr Med Chem. 2008; 15(27):2876-85. Epub 2008/11/11. doi: 10.2174/092986708786242831. PubMed PMID: 18991642.
131. An S S, Wang W C, Koziol-White C J, Ahn K, Lee D Y, Kurten R C, Panettieri R A, Jr., Liggett S B. TAS2R activation promotes airway smooth muscle relaxation despite beta(2)-adrenergic receptor tachyphylaxis. Am J Physiol Lung Cell Mol Physiol. 2012; 303(4):L304-11. doi: 10.1152/ajplung.00126.2012. PubMed PMID: 22683571; PMCID: 3423830.
132. Cooper P R, Kurten R C, Zhang J, Nicholls D J, Dainty I A, Panettieri R A. Formoterol and salmeterol induce a similar degree of beta2-adrenoceptor tolerance in human small airways but via different mechanisms. Br J Pharmacol. 2011; 163(3):521-32. doi: 10.1111/j.1476-5381.2011.01257.x. PubMed PMID: 21306583; PMCID: 3101615.
133. Manson M L, Safholm J, James A, Johnsson A K, Bergman P, Al-Ameri M, Orre A C, Karrman-Mardh C, Dahlen S E, Adner M. IL-13 and IL-4, but not IL-5 nor IL-17A, induce hyperresponsiveness in isolated human small airways. J Allergy Clin Immunol. 2020; 145(3):808-17 e2. Epub 2019/12/06. doi: 10.1016/j.jaci.2019.10.037. PubMed PMID: 31805312.
134. Kan M, Koziol-White C, Shumyatcher M, Johnson M, Jester W, Panettieri R A, Jr., Himes B E. Airway Smooth Muscle-Specific Transcriptomic Signatures of Glucocorticoid Exposure. Am J Respir Cell Mol Biol. 2019; 61(1):110-20. Epub 2019/01/30. doi: 10.1165/remb.2018-03850C. PubMed PMID: 30694689; PMCID: PMC6604213.
135. Nadiminty N, Chun J Y, Hu Y, Dutt S, Lin X, Gao A C. LIGHT, a member of the TNF superfamily, activates Stat3 mediated by NIK pathway. Biochem Biophys Res Commun. 2007; 359(2):379-84. doi: 10.1016/j.bbrc.2007.05.119. PubMed PMID: 17543278; PMCID: 2062522.
136. Su W B, Chang Y H, Lin W W, Hsieh S L. Differential regulation of interleukin-8 gene transcription by death receptor 3 (DR3) and type I TNF receptor (TNFRI). Exp Cell Res. 2006; 312(3):266-77. Epub 2005/12/06. doi: 10.1016/j.yexcr.2005.10.015. PubMed PMID: 16324699.
137. Yeh D Y, Wu C C, Chin Y P, Lu C J, Wang Y H, Chen M C. Mechanisms of human lymphotoxin beta receptor activation on upregulation of CCL5/RANTES production. Int Immunopharmacol. 2015; 28(1):220-9. Epub 2015/06/23. doi: 10.1016/j.intimp.2015.06.010. PubMed PMID: 26096887.
138. Patel D D, Kuchroo V K. Th17 Cell Pathway in Human Immunity: Lessons from Genetics and Therapeutic Interventions. Immunity. 2015; 43(6):1040-51. doi: 10.1016/j.immuni.2015.12.003. PubMed PMID: 26682981.
139. Wu N L, Huang D Y, Tsou FIN, Lin Y C, Lin W W. Syk mediates IL-17-induced CCL20 expression by targeting Act1-dependent K63-linked ubiquitination of TRAF6. J Invest Dermatol. 2015; 135(2):490-8. doi: 10.1038/jid.2014.383. PubMed PMID: 25202827.
140. Zhu L, Wu Y, Wei H, Xing X, Zhan N, Xiong H, Peng B. IL-17R activation of human periodontal ligament fibroblasts induces IL-23 p19 production: Differential involvement of NF-kappaB versus JNK/AP-1 pathways. Mol Immunol. 2011; 48(4):647-56. doi: 10.1016/j.molimm.2010.11.008. PubMed PMID: 21145111.
141. Dong Z, Yang Y, Zhang T, Li Y, Kang Q, Lei W, Cao Y, Niu X, Wang D, Tai W. siRNA-Actl inhibits the function of IL-17 on lung fibroblasts via the NF-kappaB pathway. Respiration. 2013; 86(4):332-40. doi: 10.1159/000348403. PubMed PMID: 23689683.
142. Zhu S, Pan W, Shi P, Gao H, Zhao F, Song X, Liu Y, Zhao L, Li X, Shi Y, Qian Y. Modulation of experimental autoimmune encephalomyelitis through TRAF3-mediated suppression of interleukin 17 receptor signaling. J Exp Med. 2010; 207(12):2647-62. doi: 10.1084/jem.20100703. PubMed PMID: 21078888; PMCID: PMC2989772.
143. Hwang S Y, Kim J Y, Kim K W, Park M K, Moon Y, Kim W U, Kim H Y. IL-17 induces production of IL-6 and IL-8 in rheumatoid arthritis synovial fibroblasts via NF-kappaB- and PI3-kinase/Akt-dependent pathways. Arthritis Res Ther. 2004; 6(2):R120-8. doi: 10.1186/ar1038. PubMed PMID: 15059275; PMCID: PMC400429.
144. Hattori T, Ogura N, Akutsu M, Kawashima M, Watanabe S, Ito K, Kondoh T. Gene Expression Profiling of IL-17A-Treated Synovial Fibroblasts from the Human Temporomandibular Joint. Mediators Inflamm. 2015; 2015:436067. doi: 10.1155/2015/436067. PubMed PMID: 26839464; PMCID: PMC4709758.
145. Jinnin M, Ihn H, Asano Y, Yamane K, Trojanowska M, Tamaki K. Upregulation of tenascin-C expression by IL-13 in human dermal fibroblasts via the phosphoinositide 3-kinase/Akt and the protein kinase C signaling pathways. J Invest Dermatol. 2006; 126(3): 551-60. doi: 10.1038/sj.jid.5700090. PubMed PMID: 16374482.
146. Jinnin M, Ihn H, Yamane K, Tamaki K. Interleukin-13 stimulates the transcription of the human alpha2(I) collagen gene in human dermal fibroblasts. J Biol Chem. 2004; 279(40):41783-91. doi: 10.1074/jbc.M406951200. PubMed PMID: 15271999.
147. Guo J, Yao H, Lin X, Xu H, Dean D, Zhu Z, Liu G, Sime P. IL-13 induces YY1 through the AKT pathway in lung fibroblasts. PLoS One. 2015; 10(3):e0119039. doi: 10.1371/journal.pone.0119039. PubMed PMID: 25775215; PMCID: PMC4361578.
148. Hashimoto S, Gon Y, Takeshita I, Maruoka S, Hone T. IL-4 and IL-13 induce myofibroblastic phenotype of human lung fibroblasts through c-Jun NH2-terminal kinase-dependent pathway. J Allergy Clin Immunol. 2001; 107(6):1001-8. doi: 10.1067/mai.2001.114702. PubMed PMID: 11398077.
149. Zhou X, Hu H, Huynh M L, Kotaru C, Balzar S, Trudeau J B, Wenzel S E. Mechanisms of tissue inhibitor of metalloproteinase 1 augmentation by IL-13 on TGF-beta 1-stimulated primary human fibroblasts. J Allergy Clin Immunol. 2007; 119(6):1388-97. doi: 10.1016/j.jaci.2007.02.011. PubMed PMID: 17418380.
150. Hartupee J, Liu C, Novotny M, Li X, Hamilton T. IL-17 enhances chemokine gene expression through mRNA stabilization. J Immunol. 2007; 179(6):4135-41. PubMed PMID: 17785852.
151. Azim A, Mistry H, Freeman A, Barber C, Newell C, Gove K, Thirlwall Y, Harvey M, Bentley K, Knight D, Long K, Mitchell F, Cheng Y, Varkonyi-Sepp J, Grabau W, Dennison P, Haitchi H M, Arshad S H, Djukanovic R, Wilkinson T, Howarth P, Kurukulaaratchy R J. Protocol for the Wessex AsThma CoHort of difficult asthma (WATCH): a pragmatic real-life longitudinal study of difficult asthma in the clinic. BMC Pulm Med. 2019; 19(1):99. Epub 2019/05/28. doi: 10.1186/s12890-019-0862-2. PubMed PMID: 31126281; PMCID: PMC6534885.
152. Waise S, Parker R, Rose-Zerilli M J J, Layfield D M, Wood O, West J, Ottensmeier C H, Thomas G J, Hanley C J. An optimised tissue disaggregation and data processing pipeline for characterising fibroblast phenotypes using single-cell RNA sequencing. Sci Rep. 2019; 9(1):9580. Epub 2019/07/05. doi: 10.1038/s41598-019-45842-4. PubMed PMID: 31270426; PMCID: PMC6610623.
153. Seumois G, Vijayanand P. Single-cell analysis to understand the diversity of immune cell types that drive disease pathogenesis. J Allergy Clin Immunol. 2019; 144(5):1150-3. Epub 2019/11/11. doi: 10.1016/j.jaci.2019.09.014. PubMed PMID: 31703762.
154. Rosales S L, Liang S, Engel I, Schmiedel B J, Kronenberg M, Vijayanand P, Seumois G. A Sensitive and Integrated Approach to Profile Messenger RNA from Samples with Low Cell Numbers. Methods Mol Biol. 2018; 1799:275-301. Epub 2018/06/30. doi: 10.1007/978-1-4939-7896-O_21. PubMed PMID: 29956159.
155. Patil V S, Madrigal A, Schmiedel B J, Clarke J, O'Rourke P, de Silva A D, Harris E, Peters B, Seumois G, Weiskopf D, Sette A, Vijayanand P. Precursors of human CD4(+) cytotoxic T lymphocytes identified by single-cell transcriptome analysis. Sci Immunol. 2018; 3(19). Epub 2018/01/21. doi: 10.1126/sciimmunol.aan8664. PubMed PMID: 29352091; PMCID: PMC5931334.
156. Ganesan A P, Clarke J, Wood O, Garrido-Martin E M, Chee S J, Mellows T, Samaniego-Castruita D, Singh D, Seumois G, Alzetani A, Woo E, Friedmann P S, King E V, Thomas G J, Sanchez-Elsner T, Vijayanand P, Ottensmeier C H. Tissue-resident memory features are linked to the magnitude of cytotoxic T cell responses in human lung cancer. Nat Immunol. 2017; 18(8):940-50. Epub 2017/06/20. doi: 10.1038/ni.3775. PubMed PMID: 28628092; PMCID: PMC6036910.
157. Randall L M, Amante F H, Zhou Y, Stanley A C, Haque A, Rivera F, Pfeffer K, Scheu S, Hill G R, Tamada K, Engwerda C R. Cutting edge: selective blockade of LIGHT-lymphotoxin beta receptor signaling protects mice from experimental cerebral malaria caused by Plasmodium berghei ANKA. J Immunol. 2008; 181(11):7458-62. Epub 2008/11/20. doi: 181/11/7458 [pii]. PubMed PMID: 19017933.
158. Xu Y, Flies A S, Flies D B, Zhu G, Anand S, Flies S J, Xu H, Anders R A, Hancock W W, Chen L, Tamada K. Selective targeting of the LIGHT-HVEM costimulatory system for the treatment of graft-versus-host disease. Blood. 2007; 109(9):4097-104. PubMed PMID: 17179227.

What is claimed is:

1. A method for one or more of:
a) reducing or inhibiting a fibrotic disease in a subject in need thereof;
b) treating a skin disease or inflammation in a subject in need thereof;
c) treating an autoimmune disorder in a subject in need thereof;
d) treating a respiratory disease in a subject in need thereof; or
e) reducing or inhibiting the activity of a LIGHT (p30 polypeptide) receptor and/or the activity of a TNF-like Ligand 1A (TL1A) receptor in a subject in need thereof;
comprising modulating the activity of LIGHT (p30 polypeptide) and the activity of TNF-like Ligand 1A (TL1A) in the subject in need thereof.

2. The method of claim 1, wherein modulating the activity of LIGHT and the activity of TL1A in the subject in need thereof comprises administering to the subject a sufficient amount of a first molecule that modulates the activity of LIGHT and a second molecule that modulates the activity of TL1A.

3. The method of claim 1 or claim 2, wherein modulating the activity of LIGHT and the activity of TL1A in the subject in need thereof comprises reducing, decreasing, suppressing, limiting, controlling, or inhibiting the activity of LIGHT, and reducing, decreasing, suppressing, limiting, controlling, or inhibiting the activity of TL1A.

4. The method of claim 2 or claim 3, wherein the first molecule comprises a fusion of an immunoglobulin with a) herpesvirus entry mediator (HVEM) or b) lymphotoxin beta receptor (LTβR) polypeptide.

5. The method of claim 2 or claim 3, wherein the first molecule comprises a polypeptide that binds to LIGHT, HVEM, or LTβR, a peptidomimetic that modulates LIGHT, or a small molecule that modulates LIGHT.

6. The method of claim 2 or claim 3, wherein the first molecule comprises an antibody that binds to LIGHT, an antibody that binds to HVEM, or an antibody that binds to LTβR.

7. The method of any one of the claims 2-6, wherein the second molecule comprises a fusion of death receptor 3 (DR3) with an immunoglobulin, a polypeptide that binds to DR3, a fusion of decoy receptor 3 (DcR3) with an immunoglobulin, a peptidomimetic that modulates TL1A, or a small molecule that modulates TL1A.

8. The method of any one of the claims 2-6, wherein the second molecule comprises an antibody that binds to TL1A or an antibody that binds to DR3.

9. The method of claim 6 or 8, wherein the antibody is a full-length antibody or an antigen binding fragment thereof.

10. The method of claim 9, wherein the antibody fragment is a Fab, Fab′, F(ab′)₂, Fv, Fd, single-chain Fv (scFv), trispecific (Fab₃), bispecific (Fab₂), diabody ((V_{L}-V_{H})₂ or (V_{H}-V_{L})₂), triabody (trivalent), tetrabody (tetravalent), minibody ((scFv-CH)₂), bispecific single-chain Fv (Bis-scFv), IgGdeltaCH2, scFv-Fc, or (scFv)₂-Fc.

11. The method of any one of the claim 6 or 8-10, wherein the antibody that binds to LIGHT, HVEM, or LTBR is a multispecific antibody that binds to LIGHT, HVEM, or LTBR and one or more of TL or DR3.

12. The method of any one of the claim 6 or 8-11, wherein the multispecific antibody comprises:
a) a first antigen binding domain that binds to LIGHT, HVEM, or LTβR and a second antigen binding domain that binds to TL1A;
b) a first antigen binding domain that binds to LIGHT, HVEM, or LTβR and a second antigen binding domain that binds to DR3;
c) a first antigen binding domain that binds to LIGHT and a second antigen binding domain that binds to TL1A or DR3;
d) a first antigen binding domain that binds to HVEM and a second antigen binding domain that binds to TL1A or DR3;
e) a first antigen binding domain that binds to LTβR and a second antigen binding domain that binds to TL1A or DR3;
f) a first antigen binding domain that binds to LIGHT and a second antigen binding domain that binds to TL1A;
g) a first antigen binding domain that binds to LIGHT and a second antigen binding domain that binds to DR3;
h) a first antigen binding domain that binds to HVEM and a second antigen binding domain that binds to TL1A;
i) a first antigen binding domain that binds to HVEM and a second antigen binding domain that binds to DR3;
j) a first antigen binding domain that binds to LTβR and a second antigen binding domain that binds to TL1A; or
k) a first antigen binding domain that binds to LTβR and a second antigen binding domain that binds to DR3.

13. The method of any one of the claim 6 or 8-12, wherein the multispecific antibody is a bispecific antibody, optionally a full-length antibody or an antigen binding fragment thereof.

14. The method of claim 13, wherein the fragment of the bispecific antibody is a scFv.

15. The method of any one of the claims 1-10 wherein the method comprises administering to the subject the first molecule and the second molecule.

16. The method of claim 15, wherein the first molecule and the second molecule are administered simultaneously.

17. The method of claim 15, wherein the first molecule and the second molecule are administered sequentially.

18. The method of claim 15, wherein the first molecule is administered prior to administering the second molecule.

19. The method of any one of the claim 15, 17, or 18, wherein the first molecule is administered after administering the second molecule.

20. The method of any one of the claims 2-19, wherein the first molecule is an inhibitor of LIGHT.

21. The method of any one of the claims 2-19, wherein the second molecule is an inhibitor of TL1A.

22. The method of any one of the claims 1-21, wherein the fibrotic disease comprises fibrosis of a parenchymal organ or tissue, optionally of the lung, liver, skin, kidney, brain, heart, joints, intestine, or the bone marrow.

23. The method of any one of the claims 1-22, wherein the fibrotic disease comprises interstitial lung disease (ILD), liver cirrhosis, or idiopathic pulmonary fibrosis.

24. The method of any one of the claims 1-21, wherein the skin disease or inflammation comprises atopic dermatitis, scleroderma, psoriasis, onchocercal dermatitis, nephrogenic fibrosing dermopathy, mixed connective tissue disease, scleromyxedema, keloid, sclerodactyly, or eosinophilic fasciitis.

25. The method of any one of the claims 1-21, wherein the respiratory disease comprises asthma, allergic asthma, bronchiolitis, pleuritis, chronic obstructive pulmonary disease (COPD), extrinsic bronchial asthma, allergic rhinitis, eosophageal allergy, or gastrointestinal allergy.

26. The method of any one of the claims 1-21, wherein the respiratory disease comprises nodules, eosinophilia, rheumatism, dermatitis and swelling (NERDS).

27. The method of any one of the claims 1-21, wherein the respiratory disease comprises airway obstruction, apnea, asbestosis, atelectasis, berylliosis, bronchiectasis, bronchiolitis, bronchiolitis obliterans organizing pneumonia, bronchitis, bronchopulmonarydysplasia empyema, pleural empyema, pleural epiglottitis, hemoptysis, hypertension, kartagener syndrome, meconium aspiration, pleural effusion, pleurisy, pneumonia, pneumothorax, respiratory distress syndrome, respiratory hypersensitivity, respiratory tract infections, rhinoscleroma, scimitar syndrome, severe acute respiratory syndrome, silicosis, or tracheal stenosis.

28. The method of any one of the claims 1-21, wherein the autoimmune disorder is systemic sclerosis, rheumatoid arthritis (RA), lupus (e.g., systemic lupus erythematosus or SLE), inflammatory bowel disease (IBD), eosinophilic esophagitis (EoE), ankylosing spondylitis (AS), experimental autoimmune encephalomyelitis (EAE), or an autoimmune inflammatory disease of the central nervous system (CNS).

29. The method of any one of claims 1-28, further comprising administering to the subject an additional therapeutic agent.

30. The method of claim 29, wherein the additional therapeutic agent comprises an anti-inflammatory drug, a steroid, a hormone, or an immunosuppressant drug.

31. The method of claim 29 or 30, wherein the first molecule and/or the second molecule and the additional therapeutic agent are administered simultaneously.

32. The method of claim 29 or 30, wherein the first molecule and/or the second molecule and the additional therapeutic agent are administered sequentially.

33. The method of any one of the claim 29, 30, or 32, wherein the first molecule and/or the second molecule are administered prior to administering the additional therapeutic agent.

34. The method of any one of the claim 29, 30, or 32, wherein the first molecule and/or the second molecule are administered after administering the additional therapeutic agent.

35. The method of any one of the claims 2-34, wherein the first molecule and/or the second molecule and/or the additional therapeutic agent are administered systemically.

36. The method of any one of the claims 2-34, wherein the first molecule and/or the second molecule and/or the additional therapeutic agent are administered locally.

37. The method of any one of the claims 2-36, wherein the first molecule and/or the second molecule and/or the additional therapeutic agent are administered by parenteral administration.

38. The method of any one of the claims 2-37, wherein the first molecule and/or the second molecule and/or the additional therapeutic agent are administered intravenously or subcutaneously.

39. The method of any one of the claims 1-38, wherein the subject is a mammal or a human.

40. A pharmaceutical composition comprising a first molecule that modulates the activity of LIGHT (p30 polypeptide), a second molecule that modulates the activity of TNF-like Ligand 1A (TL1A), and a pharmaceutically acceptable excipient.

41. A combination comprising a first molecule that modulates the activity of LIGHT (p30 polypeptide) and a second molecule that modulates the activity of TNF-like Ligand 1A (TL1A).

42. The combination of claim 41, further comprising a pharmaceutically acceptable excipient.

43. The combination of claim 41, wherein the first molecule comprises a fusion of an immunoglobulin and a) herpesvirus entry mediator (HVEM) or b) lymphotoxin beta receptor (LTβR) polypeptide with.

44. The combination of claim 41, wherein the first molecule comprises a polypeptide that binds to LIGHT, HVEM, or LTβR, a peptidomimetic that modulates LIGHT, or a small molecule that modulates LIGHT.

45. The combination of claim 41, wherein the first molecule comprises an antibody that binds to LIGHT, an antibody that binds to HVEM, or an antibody that binds to LTβR.

46. The combination of any one of the claims 41-45, wherein the second molecule comprises a fusion of DR3 with an immunoglobulin, a polypeptide that binds to DR3, a fusion of DcR3 with an immunoglobulin, a peptidomimetic that modulates TL1A, or a small molecule that modulates TL1A.

47. The combination of any one of the claims 41-45, wherein the second molecule comprises an antibody that binds to TL1A or an antibody that binds to DR3.

48. The combination of claim 45, wherein antibody that binds to LIGHT, HVEM, or LTBR is a multispecific antibody that binds to LIGHT, HVEM, or LTBR and one or more of TL1A or DR3.

49. The combination of claim 48, wherein the multispecific antibody comprises:
a) a first antigen binding domain that binds to LIGHT, HVEM, or LTβR and a second antigen binding domain that binds to TL1A;
b) a first antigen binding domain that binds to LIGHT, HVEM, or LTβR and a second antigen binding domain that binds to DR3;
c) a first antigen binding domain that binds to LIGHT and a second antigen binding domain that binds to TL1A or DR3;
d) a first antigen binding domain that binds to HVEM and a second antigen binding domain that binds to TL1A or DR3;
e) a first antigen binding domain that binds to LTβR and a second antigen binding domain that binds to TL1A or DR3;
f) a first antigen binding domain that binds to LIGHT and a second antigen binding domain that binds to TL1A;
g) a first antigen binding domain that binds to LIGHT and a second antigen binding domain that binds to DR3;
h) a first antigen binding domain that binds to HVEM and a second antigen binding domain that binds to TL1A;
i) a first antigen binding domain that binds to HVEM and a second antigen binding domain that binds to DR3;
j) a first antigen binding domain that binds to LTβR and a second antigen binding domain that binds to TL1A; or
k) a first antigen binding domain that binds to LTβR and a second antigen binding domain that binds to DR3.

50. The combination of any one of the claims 41-49, wherein the combination comprises a pharmaceutical composition comprising the first molecule that modulates the activity of LIGHT (p30 polypeptide), the second molecule that modulates the activity of TNF-like Ligand 1A (TL1A), and a pharmaceutically acceptable excipient.

51. The pharmaceutical composition of claim 40 or the combination of any one of the claims 41-50, wherein the first molecule is an inhibitor of LIGHT.

52. The pharmaceutical composition of claim 40 or the combination of any one of the claims 41-51, wherein the second molecule is an inhibitor of TL1A.

53. A kit comprising a first molecule that modulates the activity of LIGHT (p30 polypeptide) and/or a second molecule that modulates the activity of TNF-like Ligand 1A (TL1A) for use with the method of claims 2-39, and optionally comprises instructions for use.

54. The kit of claim 53, wherein the first molecule is an inhibitor of LIGHT.

55. The kit of claim 53 or claim 54, wherein the second molecule is an inhibitor of TL1A.
